SEQUENCE LISTING (74 sequences)

SEQ ID NO: 1 - DNA, 1345 bp, Homo sapiens
ccaccccccttcccacctgaccagccatgggggctgcggtgtttttcggctgcactttcgtcgcgttcggcccggccttcgcgcttttcttgatcactgtggctggggacccgcttcgcgttatcatcctggtcgcaggggcatttttctggctggtctccctgctcctggcctctgtggtctggttcatcttggtccatgtgaccgaccggtcagatgcccggatcctggtcgcaggggcatttttctggctggtctccctgctcctggcctctgtggtctggttcatcttggtccatgtgaccgaccggtcagatgcccggctccagtacggcctcctgatttttggtgctgctgtctctgtccttctacaggaggtgttccgctttgcctactacaagctgcttaagaaggcagatgaggggttagcatcgctgagtgaggacggaagatcacccatctccatccgccagatggcctatgtttctggtctctccttcggtatcatcagtggtgtcttctctgttatcaatattttggctgatgcacttgggccaggtgtggttgggatccatggagactcaccctattacttcctgacttcagcctttctgacagcagccattatcctgctccataccttttggggagttgtgttctttgatgcctgtgagaggagacggtactgggctttgggcctggtggttgggagtcacctactgacatcgggactgacattcctgaacccctggtatgaggccagcctgctgcccatctatgcagtcactgtttccatggggctctgggccttcatcacagctggagggtccctccgaagtattcagcgcagctcttgtgtaaggactgactacctggactgatcgcctgacagatcccacctgcctgtccactgcccatgactgagcccagccccagcccggggtccattgcccacattctctgtctccttctcgtcggtctaccccactacctccagggttttgctttgtccttttgtgaccgttagtctctaagctttaccaggagcagcctgggttcagccagtcagtgactggtgggtttgaatctgcacttatccccaccacctggggacccccttgttgtgtccaggactccccctgtgtcagtgctctgctctcaccctgcccaagactcacctcccttcccctctgcaggccgacggcaggaggacagtcgggtgatggtgtattctgccctgcgcatcccacccgaggactgagggaacctaggggggacccctgggcctggggtgccctcctgatgtcctcgccctgtatttctccatctccagttctggacagtg

SEQ ID NO: 2 - DNA, 1346 bp, Homo sapiens
ccaccccccttcccacctgaccagccatgggggctgcggtgtttttcggctgcactttcgtcgcgttcggcccggccttcgcgcttttcttgatcactgtggctggggacccgcttcgcgttatcatcctggtcgcaggggcatttttctggctggtctccctgctcctggcctctgtggtctggttcatcttggtccatgtgaccgaccggtcagatgcccggatcctggtcgcaggggcatttttctggctggtctccctgctcctggcctctgtggtctggttcatcttggtccatgtgaccgaccggtcagatgcccggctccagtacggcctcctgatttttggtgctgctgtctctgtccttctacaggaggtgttccgctttgcctactacaagctgcttaagaaggcagatgaggggttagcatcgctgagtgaggacggaagatcacccatctccatccgccagatggcctatgtttctggtctctccttcggtatcatcagtggtgtcttctctgttatcaatattttggctgatgcacttgggccaggtgtggttgggatccatggagactcaccctattacttcctgacttcagcctttctgacagcagccattatcctgctccataccttttggggagttgtgttctttgatgcctgtgagaggagacggtactgggctttgggcctggtggttgggagtcacctactgacatcgggactgacattcctgaacccctggtatgaggccagcctgctgcccatctatgcagtcactgtttccatggggctctgggccttcatcacagctggagggtccctccgaagtattcagcgcagcctcttgtgtaaggactgactacctggactgatcgcctgacagatcccacctgcctgtccactgcccatgactgagcccagccccagcccggggtccattgcccacattctctgtctccttctcgtcggtctaccccactacctccagggttttgctttgtccttttgtgaccgttagtctctaagctttaccaggagcagcctgggttcagccagtcagtgactggtgggtttgaatctgcacttatccccaccacctggggacccccttgttgtgtccaggactccccctgtgtcagtgctctgctctcaccctgcccaagactcacctcccttcccctctgcaggccgacggcaggaggacagtcgggtgatggtgtattctgccctgcgcatcccacccgaggactgagggaacctaggggggacccctgggcctggggtgccctcctgatgtcctcgccctgtatttctccatctccagttctggacagtg

SEQ ID NO: 3 - DNA, 1007 bp, Homo sapiens
ccaccccccttcccacctgaccagccatgggggctgcggtgtttttcggctgcactttcgtcgcgttcggcccggccttcgcgcttttcttgatcactgtggctggggacccgcttcgcgttatcatcctggtcgcaggggcatttttctggctggtctccctgctcctggcctctgtggtctggttcatcttggtccatgtgaccgaccggtcagatgcccggatcctggtcgcaggggcatttttctggctggtctccctgctcctggcctctgtggtctggttcatcttggtccatgtgaccgaccggtcagatgcccggctccagtacggcctcctgatttttggtgctgctgtctctgtccttctacaggaggtgttccgctttgcctactacaagctgcttaagaaggcagatgaggggttagcatcgctgagtgaggacggaagatcacccatctccatccgccagatggcctatgtttctggtctctccttcggtatcatcagtggtgtcttctctgttatcaatattttggctgatgcacttgggccaggtgtggttgggatccatggagactcaccctattacttcctgacttcagcctttctgacagcagccattatcctgctccataccttttggggagttgtgttctttgatgcctgtgagaggagacggtactgggctttgggcctggtggttgggagtcacctactgacatcgggactgacattcctgaacccctggtatgaggccagcctgctgcccatctatgcagtcactgtttccatggggctctgggccttcatcacagctggagggtccctccgaagtattcagcgcagcctcttgtgccgacggcaggaggacagtcgggtgatggtgtattctgccctgcgcatcccacccgaggactgagggaacctaggggggacccctgggcctggggtgccctcctgatgtcctcgccctgtatttctccatctccagttctggacagtg

SEQ ID NO: 4 - protein, 254 aa, Homo sapiens
MGAAVFFGCTFVAFGPAFALFLITVAGDPLRVIILVAGAFFWLVSLLLASVVWFILVHVTDRSDARLQYGLLIFGAAVSVLLQEVFRFAYYKLLKKADEGLASLSEDGRSPISIRQMAYVSGLSFGIISGVFSVINILADALGPGVVGIHGDSPYYFLTSAFLTAAIILLHTFWGVVFFDACERRRYWALGLVVGSHLLTSGLTFLNPWYEASLLPIYAVTVSMGLWAFITAGGSLRSIQRSSCVRTDYLDPSF

SEQ ID NO: 5 - protein, 247 aa, Homo sapiens
MGAAVFFGCTFVAFGPAFALFLITVAGDPLRVIILVAGAFFWLVSLLLASVVWFILVHVTDRSDARLQYGLLIFGAAVSVLLQEVFRFAYYKLLKKADEGLASLSEDGRSPISIRQMAYVSGLSFGIISGVFSVINILADALGPGVVGIHGDSPYYFLTSAFLTAAIILLHTFWGVVFFDACERRRYWALGLVVGSHLLTSGLTFLNPWYEASLLPIYAVTVSMGLWAFITAGGSLRSIQRSLLCKD

SEQ ID NO: 6 - protein, 269 aa, Homo sapiens
MGAAVFFGCTFVAFGPAFALFLITVAGDPLRVIILVAGAFFWLVSLLLASVVWFILVHVTDRSDARLQYGLLIFGAAVSVLLQEVFRFAYYKLLKKADEGLASLSEDGRSPISIRQMAYVSGLSFGIISGVFSVINILADALGPGVVGIHGDSPYYFLTSAFLTAAIILLHTFWGVVFFDACERRRYWALGLVVGSHLLTSGLTFLNPWYEASLLPIYAVTVSMGLWAFITAGGSLRSIQRSLLCRRQEDSRVMVYSALRIPPEDPSFB

SEQ ID NO: 7 - DNA, 1923 bp, Homo sapiens
cccctcccatttgcctgtcctggtcaggcccccaccccccttcccacctgaccagccatgggggctgcggtgtttttcggctgcactttcgtcgcgttcggcccggccttcgcgcttttcttgatcactgtggctggggacccgcttcgcgttatcatcctggtcgcaggggcatttttctggctggtctccctgctcctggcctctgtggtctggttcatcttggtccatgtgaccgaccggtcagatgcccggctccagtacggcctcctgatttttggtgctgctgtctctgtccttctacaggaggtgttccgctttgcctactacaagctgcttaagaaggcagatgaggggttagcatcgctgagtgaggacggaagatcacccatctccatccgccagatggcctatgtttctggtctctccttcggtatcatcagtggtgtcttctctgttatcaatattttggctgatgcacttgggccaggtgtggttgggatccatggagactcaccctattacttcctgacttcagcctttctgacagcagccattatcctgctccataccttttggggagttgtgttctttgatgcctgtgagaggagacggtactgggctttgggcctggtggttgggagtcacctactgacatcgggactgacattcctgaacccctggtatgaggccagcctgctgcccatctatgcagtcactgtttccatggggctctgggccttcatcacagctggagggtccctccgaagtattcagcgcagctcttgtgtaaggactgactacctggactgatcgcctgacagatcccacctgcctgtccactgcccatgactgagcccagccccagcccggggtccattgcccacattctctgtctccttctcgtcggtctaccccactacctccagggttttgctttgtccttttgtgaccgttagtctctaagctttaccaggagcagcctgggttcagccagtcagtgactggtgggtttgaatctgcacttatccccaccacctggggacccccttgttgtgtccaggactccccctgtgtcagtgctctgctctcaccctgcccaagactcacctcccttcccctctgcaggccgacggcaggaggacagtcgggtgatggtgtattctgccctgcgcatcccacccgaggactgagggaacctaggggggacccctgggcctggggtgccctcctgatgtcctcgccctgtatttctccatctccagttctggacagtgcaggttgccaagaaaagggacctagtttagccattgccctggagatgaaattaatggaggctcaaggatagatgagctctgagtttctcagtactccctcaagactggacatcttggtctttttctcaggcctgagggggaaccatttttggtgtgataaataccctaaactgcctttttttcttttttgaggtggggggagggaggaggtatattggaactcttctaacctccttgggctatattttctctcctcgagttgctcctcatggctgggctcatttcggtccctttctccttggtcccagaccttgggggaaaggaaggaagtgcatgtttgggaactggcattactggaactaatggttttaacctccttaaccaccagcatccctcctctccccaaggtgaagtggagggtgctgtggtgagctggccactccagagctgcagtgccactggaggagtcagactaccatgacatcgtagggaaggaggggagatttttttgtagtttttaattggggtgtgggaggggcggggaggttttctataaactgtatcattttctgctgagggtggagtgtcccatccttttaatcaaggtgattgtgattttgactaataaaaaagaatttgtaaaaaa

SEQ ID NO: 8 - protein, 251 aa, Homo sapiens
MGAAVFFGCTFVAFGPAFALFLITVAGDPLRVIILVAGAFFWLVSLLLASVVWFILVHVTDRSDARLQYGLLIFGAAVSVLLQEVFRFAYYKLLKKADEGLASLSEDGRSPISIRQMAYVSGLSFGIISGVFSVINILADALGPGVVGIHGDSPYYFLTSAFLTAAIILLHTFWGVVFFDACERRRYWALGLVVGSHLLTSGLTFLNPWYEASLLPIYAVTVSMGLWAFITAGGSLRSIQRSSCVRTDYLD

SEQ ID NO: 9 - DNA, 1273 bp, Homo sapiens
cccctcccatttgcctgtcctggtcaggcccccaccccccttcccacctgaccagccatgggggctgcggtgtttttcggctgcactttcgtcgcgttcggcccggccttcgcgcttttcttgatcactgtggctggggacccgcttcgcgttatcatcctggtcgcaggggcatttttctggctggtctccctgctcctggcctctgtggtctggttcatcttggtccatgtgaccgaccggtcagatgcccggctccagtacggcctcctgatttttggtgctgctgtctctgtccttctacaggaggtgttccgctttgcctactacaagctgcttaagaaggcagatgaggggttagcatcgctgagtgaggacggaagatcacccatctccatccgccagatggcctatgtttctggtctctccttcggtatcatcagtggtgtcttctctgttatcaatattttggctgatgcacttgggccaggtgtggttgggatccatggagactcaccctattacttcctgacttcagcctttctgacagcagccattatcctgctccataccttttggggagttgtgttctttgatgcctgtgagaggagacggtactgggctttgggcctggtggttgggagtcacctactgacatcgggactgacattcctgaacccctggtatgaggccagcctgctgcccatctatgcagtcactgtttccatggggctctgggccttcatcacagctggagggtccctccgaagtattcagcgcagcctcttgtgtaaggactgactacctggactgatcgcctgacagatcccacctgcctgtccactgcccatgactgagcccagccccagcccgggtccattgcccacattctctgtctccttctcgtcggtctaccccactacctccagggttttgctttgtccttttgtgaccgttagtctctaagctttaccaggagcagcctgggttcagccagtcagtgactggtgggtttgaatctgcacttatccccaccacctggggacccccttgttgtgtccaggactccccctgtgtcagtgctctgctctcaccctgcccaagactcacctcccttcccctctgcaggccgacggcaggaggacagtcgggtgatggtgtattctgccctgcgcatcccacccgaggactgagggaacctaggggggacccctgggcctggggtgccctcctgatgtcctcgccctgtatttctccatctccagttctggac

SEQ ID NO: 10 - protein, 247 aa, Homo sapiens; identical to SEQ ID NO: 5

SEQ ID NO: 11 - DNA, 939 bp, Homo sapiens
cccctcccatttgcctgtcctggtcaggcccccaccccccttcccacctgaccagccatgggggctgcggtgtttttcggctgcactttcgtcgcgttcggcccggccttcgcgcttttcttgatcactgtggctggggacccgcttcgcgttatcatcctggtcgcaggggcatttttctggctggtctccctgctcctggcctctgtggtctggttcatcttggtccatgtgaccgaccggtcagatgcccggctccagtacggcctcctgatttttggtgctgctgtctctgtccttctacaggaggtgttccgctttgcctactacaagctgcttaagaaggcagatgaggggttagcatcgctgagtgaggacggaagatcacccatctccatccgccagatggcctatgtttctggtctctccttcggtatcatcagtggtgtcttctctgttatcaatattttggctgatgcacttgggccaggtgtggttgggatccatggagactcaccctattacttcctgacttcagcctttctgacagcagccattatcctgctccataccttttggggagttgtgttctttgatgcctgtgagaggagacggtactgggctttgggcctggtggttgggagtcacctactgacatcgggactgacattcctgaacccctggtatgaggccagcctgctgcccatctatgcagtcactgtttccatggggctctgggccttcatcacagctggagggtccctccgaagtattcagcgcagcctcttgtgccgacggcaggaggacagtcgggtgatggtgtattctgccctgcgcatcccacccgaggactgagggaacctaggggggacccctgggcctggggtgccctcctgatgtcctcgccctgtatttctccatctccagttctggacagtg

SEQ ID NO: 12 - protein, 265 aa, Homo sapiens
MGAAVFFGCTFVAFGPAFALFLITVAGDPLRVIILVAGAFFWLVSLLLASVVWFILVHVTDRSDARLQYGLLIFGAAVSVLLQEVFRFAYYKLLKKADEGLASLSEDGRSPISIRQMAYVSGLSFGIISGVFSVINILADALGPGVVGIHGDSPYYFLTSAFLTAAIILLHTFWGVVFFDACERRRYWALGLVVGSHLLTSGLTFLNPWYEASLLPIYAVTVSMGLWAFITAGGSLRSIQRSLLCRRQEDSRVMVYSALRIPPED

SEQ ID NO: 13 - DNA, 885 bp, Homo sapiens
tttccgcggtggccatgactgcggccgtgttcttcggctgcgccttcattgccttcgggcctgcgctcgccctttatgtcttcaccatcgccaccgagccgttgcgtatcatcttcctcatcgccggagctttcttctggttggtgtctctactgatttcgtcccttgtttggttcatggcaagagtcattattgacaacaaagatggaccaacacagaaatatctgctgatctttggagcgtttgtctctgtctatatccgagaaatgttccgatttgcatattataaactcttaaaaaaagccagtgaaggtttgaagagtataaacccaggtgagacagcaccctctatgcgactgctggcctatgtttctggcttgggctttggaatcatgagtggagtattttcctttgtgaataccctatctgactccttggggccaggcacagtgggcattcatggagattctcctcaattcttcctttattcagctttcatgacgctggtcattatcttgctgcatgtattctggggcattgtattttttgatggctgtgagaagaaaaagtggggcatcctccttatcgttctcctgacccacctgctggtgtcagcccagaccttcataagttcttattatggaataaacctggcgtcagcatttataatcctggtgctcatgggcacctgggcattcttagctgcgggaggcagctgccgaagcctgaaactctgcctgctctgccaagacaagaactttcttctttacaaccagcgctccagataacctcagggaaccagcacttcccaaaccgcagactacatctttagaggaagcacaactgtgcctttttctgaaaatccctttttctggtggaaaaaaa

SEQ ID NO: 14 - protein, 257 aa, Homo sapiens
MTAAVFFGCAFIAFGPALALYVFTIATEPLRIIFLIAGAFFWLVSLLISSLVWFMARVIIDNKDGPTQKYLLIFGAFVSVYIREMFRFAYYKLLKKASEGLKSINPGETAPSMRLLAYVSGLGFGIMSGVFSFVNTLSDSLGPGTVGIHGDSPQFFLYSAFMTLVIILLHVFWGIVFFDGCEKKKWGILLIVLLTHLLVSAQTFISSYYGINLASAFIILVLMGTWAFLAAGGSCRSLKLCLLCQDKNFLLYNQRSR

SEQ ID NO: 15 - DNA, 762 bp, Drosophila melanogaster
cagtaataatacaaagacaagatgacgttgcccgagttctttggctgcaccttcatcgccttcggaccgcccttcgccttgttcgtcttcaccatcgccaatgatccagtgcggatcatcattctgattgcggcggcattcttctggctgctttccctgctgatctcttccctgtggtatgccctgattccgctgaaggagttcctggcatttggcgtggtcttctcggtgtgcttccaggaagccttccggtacatcatctaccggatactgcgcagcacggagcagggattgcacgccgtggcggaggacacgcgagtgacggacaacaagcacatcctggcctatgtctccggcttgggattcggcattatatccgggatgtttgcactggtcaatgtgctggctgatatgagtggtcccggcaccatgggcttgaagggcggaactgagctattcttcgtcacctcggctgcccaggcgttgtcgattatcctgctgcacaccttctggagcgttattttcttcaacgcattcgacacaaacaactatatccacataggctatgtggttttcagccacctgttcgtctccctgataactctgctcaatgccaatgagctttacacgaccactctgctgataaactacttggtcaccatacttacgggagtcctggccttccgggtggctggaggaacatctcgcagtttcagaaaattcataacatgccagtaaacatactcctagtattaaccgcct

SEQ ID NO: 16 - protein, 238 aa, Drosophila melanogaster
MTLPEFFGCTFIAFGPPFALFVFTIANDPVRIIILIAAAFFWLLSLLISSLWYALIPLKEFLAFGVVFSVCFQEAFRYIIYRILRSTEQGLHAVAEDTRVTDNKHILAYVSGLGFGIISGMFALVNVLADMSGPGTMGLKGGTELFFVTSAAQALSIILLHTFWSVIFFNAFDTNNYIHIGYVVFSHLFVSLITLLNANELYTTTLLINYLVTILTGVLAFRVAGGTSRSFRKFITCQ

SEQ ID NO: 17 - DNA, 2301 bp, Homo sapiens
atgggggctgcggtgtttttcggctgcactttcgtcgcgttcggcccggccttcgcgcttttcttgatcactgtggctggggacccgcttcgcgttatcatcctggtcgcagggtgagtagagggcccgggagacgcgggagagcgtcgaagagagaggtgcggaaggggctggaggaactggggcaagcctgggagcctgaattggggacgataagtcggaggtgaagtttgggcggaggtgaggggttgggtctgggagatttgtcctttcccgcagttggtttccaccttccaaggatctcacagattcctcctatattcctcccagcgacgtcagagaaggcccaaggccgagactcgtgagggggctgtgctgacctaggcaggccgagtcaggtgccttaggggaggatccaggaacggatacctcgcccttccgtgctcgcacactctggctgtcatcgctctgaagactctttaattagatttctcccctttccagtgcgttcacttttctacagatgagtctcttggtggagacagttaccctacctggtccatgtctccctaaccatccggaaggctaacttccacttttcaagcagctttggctggtttccctccttgatttctctggctcccactactattgcttgtctcactgcccctgtcttttctcagggcatttttctggctggtctccctgctcctggcctctgtggtctggttcatcttggtccatgtgaccgaccggtcagatgcccggctccagtacggcctcctgatttttggtgctgctgtctctgtccttctacaggaggtgttccgctttgcctactacaagctgcttaagtaagaagatggagtggtctggaggggagaggggcaaaggactgcactatgggaagtggggcagcccctgggtgctggtttggaagaggaggcactaagggaggacattagagggaaaggagcatccctgccctccctcatgtttcccctaccccaccccaccccaggaaggcagatgaggggttagcatcgctgagtgaggacggaagatcacccatctccatccgccagatggcctatggtgagccaagggagagggactggaggagggagttggacagccccctcctctagggaagtctctaaatatccacatgttctaagtggcttcttactttccttcatccgtcacttccaaagaaagttggtctggagggagagtagatgtgaaagaattgtaaccgggaatggggaggggtcagtggtgaacaggcaatagtgtgatctctgacattgatgagatcctcccttcccccagtttctggtctctccttcggtatcatcagtggtgtcttctctgttatcaatattttggctgatgcacttgggccaggtgtggttgggatccatggagactcaccctattacttcctgacttcaggtaagatccaccttctatctagcctttaccccccatccatccttgtccctgatctgatttattggccttccctgagagacttctttggctcaacatctcaggagcctgggagaagatcagggatgtatctcctccccatctccctccctgcagcctttctgacagcagccattatcctgctccataccttttggggagttgtgttctttgatgcctgtgagaggagacggtactgggctttgggcctggtggttgggagtcacctactgacatcgggactggtgagttggagacaggggcctgagttagggagaaaagcatttaatggtgagtgggatgtgggggaaagggtatcctcacttcttaacatttttaacttacctgggaggaggaggaaaggtgagtctttcaaggtctctcacctcagcatcatttctatcacctgctctggggaggaggttgaaaggattagtcaaactgtaatgcagagggcctgaggtgagcaggagcggcagaaacctttgagtttctgaggagctgaaaatcaaaagtccccttaaccacaagatgttggtgctctgaagggaaagactggagaatttgagagagatatctgggagtcagaaaggtacagagagaatatggggattaggtcgagggagaatctaatctctttcctactcttaccctccttcctagacattcctgaacccctggtatgaggccagcctgctgcccatctatgcagtcactgtttccatggggctctgggccttcatcacagctggagggtccctccgaagtattcagcgcagcctcttgtgtaaggactgactacctggactg

SEQ ID NO: 18 - protein, 1200 aa, Mus musculus
TTCCCTCCCTTCCCCAGCTGCCCAGTCATGGGGGCTGCTGTGTTTTTCGGATGCACCTTCGTCGCGTTCGGCCCAGCCTTCTCCCTTTTCCTGATCACTGTAGCTGGAGACCCACTTCGGGTTATCATCCTGGTGGCGGGAGCCTTTTTCTGGCTGGTCTCCCTGCTCTTGGCTTCTGTGGTCTGGTTCATCTTGGTCCATGTGACAGACCGATCAGATGCACGGCTCCAGTATGGCCTCCTGATTTTTGGTGCTGCTGTCTCTGTCCTTCTACAGGAAGTGTTCCGTTTTGCTTACTACAAGCTCCTTAAGAAGGCAGATGAGGGCTTAGCATCACTGAGTGAGGACGGAAGATCACCCATCTCCATCCGACAGATGGCCTATGTTTCTGGTCTGTCCTTCGGTATCATCAGTGGTGTCTTCTCTGTTATCAATATTTTGGCTGATGCACTTGGGCCAGGTGTGGTTGGGATCCATGGAGACTCACCCTATTACTTCCTGACTTCAGCCTTTCTGACAGCAGCCATTATCCTGCTCCACACCTTTTGGGGAGTTGTGTTCTTTGATGCCTGTGAGAGGAGACGGTACTGGGCTTTGGGCCTGGTAGTTGGGAGTCACCTTCTGACATCGGGACTGACATTCCTGAACCCCTGGTATGAGGCTAGCCTGCTGCCCATCTATGCAGTCACCGTTTCCATGGGGCTCTGGGCGTTCATCACAGCCGGAGGCTCCCTCCGAAGTATCCAGCGCAGCCTTTCGTGTAAGGACTGACTACCTGGACTGATCGCCCGACAGATCCCATCTGCCTATCCACTGCCCATGACTGAACCCAGCCCCAGCCCGGGTCCATTGCCCTCATCCTCCGTCTCCTCGCTGATGTGCCCCGCTTCCTTCCGGGTTTGGCGTTGTCCATTTGTGACCTGTAGTCTCTAAGCTTTCTCAGGAGCAGCCTGGGTGCAGCCAGTCAGGGACTGGTGGGTTTGAATCTGCATCTCTCCCCACCACCTGGGGACCCCCTTGTTGTCCAGGTCTCCCCATGTGTCAGTGCTCCACCCTCACCCTGCCCATGACTCACCCCGCTTCCCCTCTGCAGGCCGCCGGCAGGAGGACAGTCGGGTGATGGTGTACTCTGCCCTGCGCATCCCACCCGAGGACTGAGGGAACATGGGGGGGCCCCTGGGCCTGGGGTGCCCTCCCGAT

SEQ ID NO: 19 - protein, 247 aa, Mus musculus
MGAAVFFGCTFVAFGPAFSLFLITVAGDPLRVIILVAGAFFWLVSLLLASVVWFILVHVTDRSDARLQYGLLIFGAAVSVLLQEVFRFAYYKLLKKADEGLASLSEDGRSPISIRQMAYVSGLSFGIISGVFSVINILADALGPGVVGIHGDSPYYFLTSAFLTAAIILLHTFWGVVFFDACERRRYWALGLVVGSHLLTSGLTFLNPWYEASLLPIYAVTVSMGLWAFITAGGSLRSIQRSLSCKD

SEQ ID NO: 20 - DNA, 867 bp, Mus musculus
ttccctcccttccccagctgcccagtcatgggggctgctgtgtttttcggatgcaccttcgtcgcgttcggcccagccttctcccttttcctgatcactgtagctggagacccacttcgggttatcatcctggtggcgggagcctttttctggctggtctccctgctcttggcttctgtggtctggttcatcttggtccatgtgacagaccgatcagatgcacggctccagtatggcctcctgatttttggtgctgctgtctctgtccttctacaggaagtgttccgttttgcttactacaagctccttaagaaggcagatgagggcttagcatcactgagtgaggacggaagatcacccatctccatccgacagatggcctatgtttctggtctgtccttcggtatcatcagtggtgtcttctctgttatcaatattttggctgatgcacttgggccaggtgtggttgggatccatggagactcaccctattacttcctgacttcagcctttctgacagcagccattatcctgctccacaccttttggggagttgtgttctttgatgcctgtgagaggagacggtactgggctttgggcctggtagttgggagtcaccttctgacatcgggactgacattcctgaacccctggtatgaggctagcctgctgcccatctatgcagtcaccgtttccatggggctctgggcgttcatcacagccggaggctccctccgaagtatccagcgcagcctttcgtgccgccggcaggaggacagtcgggtgatggtgtactctgccctgcgcatcccacccgaggactgagggaacatgggggggcccctgggcctggggtgccctcccgat

SEQ ID NO: 21 - protein, 265 aa, Mus musculus
MGAAVFFGCTFVAFGPAFSLFLITVAGDPLRVIILVAGAFFWLVSLLLASVVWFILVHVTDRSDARLQYGLLIFGAAVSVLLQEVFRFAYYKLLKKADEGLASLSEDGRSPISIRQMAYVSGLSFGIISGVFSVINILADALGPGVVGIHGDSPYYFLTSAFLTAAIILLHTFWGVVFFDACERRRYWALGLVVGSHLLTSGLTFLNPWYEASLLPIYAVTVSMGLWAFITAGGSLRSIQRSLSCRRQEDSRVMVYSALRIPPED

SEQ ID NO: 22 - DNA, 92 bp, Artificial sequences
  primer_bind (1)..(92): PCR primer
gactgcgatatcatgaagttatgcatattactggccgtcgtggcctttgttggcctctcgctcggggatgcagaattccgacatgactcagg

SEQ ID NO: 23 - DNA, 77 bp, Artificial sequences
  primer_bind (1)..(77): PCR primer
gccgactctagactagaggcttgcataatctggcacatcatatggatagttctgcatctgctcaaagaacttgtagg

SEQ ID NO: 24 - DNA, 41 bp, Artificial sequences
  primer_bind (1)..(41): PCR primer
cccgggggtacctcttcgatggaaatgcgtctgaatgcggc

SEQ ID NO: 25 - DNA, 36 bp, Artificial sequences
  primer_bind (1)..(36): PCR primer
aaatttgaattcaccaaataatgcggcattgcttgc

SEQ ID NO: 26 - DNA, 30 bp, Artificial sequences
  primer_bind (1)..(30): PCR primer
gccatgggggctgcggtgtttttcggctgc

SEQ ID NO: 27 - DNA, 32 bp, Artificial sequences
  primer_bind (1)..(27): PCR primer
gtccaggtagtcagtccttacacaagagctgc

SEQ ID NO: 28 - DNA, 32 bp, Artificial sequences
  primer_bind (1)..(32): PCR primer
gtccttacacaagaggctgcgctgaatacttc

SEQ ID NO: 29 - DNA, 31 bp, Artificial sequences
  primer_bind (1)..(31): PCR primer
gtcctcgggtgggatgcgcagggcagaatac

SEQ ID NO: 30 - DNA, 27 bp, Artificial sequences
  primer_bind (1)..(27): PCR primer
gaattaatacgactcactatagggaga

SEQ ID NO: 31 - DNA, 23 bp, Artificial sequences
  primer_bind (1)..(23): PCR primer
atttaggtgacactatagaagcg

SEQ ID NO: 32 - DNA, 31 bp, Artificial sequences
  primer_bind (1)..(31): PCR primer
gggctcgcctctgaggatgacgccaatgtgg

SEQ ID NO: 33 - DNA, 32 bp, Artificial sequences
  primer_bind (1)..(32): PCR primer
aatgcggaggggtcctcttggcgaaaggacag

SEQ ID NO: 34 - DNA, 34 bp, Artificial sequences
  primer_bind (1)..(34): PCR primer
atggaaatgcgtctgaatgcggcttccatatggc

SEQ ID NO: 35 - DNA, 31 bp, Artificial sequences
  primer_bind (1)..(31): PCR primer
ttcgtatggactgtctccaaagttgtttccg

SEQ ID NO: 36 - DNA, 33 bp, Artificial sequences
  primer_bind (1)..(33): PCR primer
gatgcagaatccgacatgactcaggatatgaag

SEQ ID NO: 37 - DNA, 33 bp, Artificial sequences
  primer_bind (1)..(33): PCR primer
gttctgcatctgctcaaagaacttgtaggttgg

SEQ ID NO: 38 - DNA, 29 bp, Artificial sequences
  primer_bind (1)..(29): PCR primer
cacattggccaagctggtgtccagatcgg

SEQ ID NO: 39 - DNA, 28 bp, Artificial sequences
  primer_bind (1)..(28): PCR primer
gcgcaacgaggccgtaatggaggaaacg

SEQ ID NO: 40 - DNA, 30 bp, Artificial sequences
  primer_bind (1)..(30): PCR primer
cagctgaaacgaacctttcttggcttcgac

SEQ ID NO: 41 - DNA, 30 bp, Artificial sequences
  primer_bind (1)..(30): PCR primer
caaatcgggtgcacaaacatttaggcattc

SEQ ID NO: 42 - DNA, 31 bp, Artificial sequences
  primer_bind (1)..(31): PCR primer
gctatgtcgctatcaagcctgctgcccacgc

SEQ ID NO: 43 - DNA, 34 bp, Artificial sequences
  primer_bind (1)..(34): PCR primer
gaatgtactgggcgggacccgccttctgggcatg

SEQ ID NO: 44 - DNA, 34 bp, Artificial sequences
  primer_bind (1)..(34): PCR primer
cggatgctgacgacgtcttcaagcacaatgcttg

SEQ ID NO: 45 - DNA, 34 bp, Artificial sequences
  primer_bind (1)..(34): PCR primer
ccaggtcgtatcgtccgtagtcgcggaagaggag

SEQ ID NO: 46 - DNA, 34 bp, Artificial sequences
  primer_bind (1)..(34): PCR primer
ttaaatatgtctgacaacaacagcggatccggag

SEQ ID NO: 47 - DNA, 35 bp, Artificial sequences
  primer_bind (1)..(35): PCR primer
ttaacataacgctcatagaactccagcgaactaac

SEQ ID NO: 48 - DNA, 26 bp, Artificial sequences
  primer_bind (1)..(26): PCR primer
ccggaaatatcatcatttcaggcagc

SEQ ID NO: 49 - DNA, 27 bp, Artificial sequences
  primer_bind (1)..(27): PCR primer
gtcgaggaccattagctttgtttcctc

SEQ ID NO: 50 - DNA, 28 bp, Artificial sequences
  primer_bind (1)..(28): PCR primer
tcgcataataatcaatacaatccacctg

SEQ ID NO: 51 - DNA, 27 bp, Artificial sequences
  primer_bind (1)..(27): PCR primer
gatgccgccactcttgaagatggccag

SEQ ID NO: 52 - DNA, 34 bp, Artificial sequences
  primer_bind (1)..(34): PCR primer
gatgacgttgcccgagttctttggctgcaccttc

SEQ ID NO: 53 - DNA, 32 bp, Artificial sequences
  primer_bind (1)..(32): PCR primer
aatactaggagtatgtttactggcatgttatg

SEQ ID NO: 54 - DNA, 23 bp, Artificial sequences
  primer_bind (1)..(23): PCR primer
taatacgactcactatagggaga

SEQ ID NO: 55 - DNA, 23 bp, Artificial sequences
  primer_bind (1)..(23): PCR primer
atttaggtgacactatagaagcg

SEQ ID NO: 56 - RNA, 23 nt, Artificial sequences
  misc_RNA (1)..(23): sense RNA
gccaucaugaucagugucauuaa

SEQ ID NO: 57 - RNA, 23 nt, Artificial sequences
  misc_RNA (1)..(23): antisense RNA
aaugacacugaucaugauggcug

SEQ ID NO: 58 - RNA, 23 nt, Artificial sequences
  misc_RNA (1)..(23): sense RNA
cacccucaucaugaucagcguaa

SEQ ID NO: 59 - RNA, 23 nt, Artificial sequences
  misc_RNA (1)..(23): antisense RNA
acgcugaucaugaugagggugug

SEQ ID NO: 60 - RNA, 23 nt, Artificial sequences
  misc_RNA (1)..(23): sense RNA
ccugcucaacgccacucaucaaa

SEQ ID NO: 61 - RNA, 23 nt, Artificial sequences
  misc_RNA (1)..(23): antisense RNA
ugaugaguggcguugagcaggug

SEQ ID NO: 62 - RNA, 23 nt, Artificial sequences
  misc_RNA (1)..(23): sense RNA
uagaugagggcaugcagauccaa

SEQ ID NO: 63 - RNA, 23 nt, Artificial sequences
  misc_RNA (1)..(23): antisense RNA
ggaucugcaugcccucaucuaug

SEQ ID NO: 64 - RNA, 23 nt, Artificial sequences
  misc_RNA (1)..(23): sense RNA
cggcugcacuuucgucgcguuaa

SEQ ID NO: 65 - RNA, 23 nt, Artificial sequences
  misc_RNA (1)..(23): antisense RNA
aacgcgacgaaagugcagccgug

SEQ ID NO: 66 - RNA, 23 nt, Artificial sequences
  misc_RNA (1)..(23): sense RNA
cgucccuuguuugguucauggaa

SEQ ID NO: 67 - RNA, 23 nt, Artificial sequences
  misc_RNA (1)..(23): antisense RNA
ccaugaaccaaacaagggacgug

SEQ ID NO: 68 - DNA, 30 bp, Artificial sequences
  primer_bind (1)..(30): PCR primer
ccaccccccttcccacctgaccagccatgg

SEQ ID NO: 69 - DNA, 30 bp, Artificial sequences
  primer_bind (1)..(30): PCR primer
cactgtccagaactggagatggagaaatac

SEQ ID NO: 70 - protein, 258 aa, Mus musculus
MTLPVFFGCAFIAFGPAFALYLFTIATDPLRVIFLIAGAFFWLVSLLLSSMFWFLVRVITNNRDESVQNYLLIFGALLSVCIQELFRLAYYKLLKKASEGLKSINPEEDIAPSMRLLAYVSGLGFGIMSGVFSFVNTLSNSLGPGTVGIHGDSPQFFLNSAFMTLVVIMLHVFWGVVFFDGCEKNKWYTLLTVLLTHLVVSTQTFLSPYYEVNLVTAYIIMVLMGIWAFYVAGGSCRSLKFCLLCQDKDFLLYNQRSR

SEQ ID NO: 71 - DNA, 2563 bp, Drosophila melanogaster
ggccggctgccttgcctttcgaaagtcagttgcgtgcgagccgcgagcgcgagatcatcaaactgagaaagtcggactgcgactcgaaactgaaattgaaactgaaagagagaaatattcaaattgtcgtgtgtgtgggtgcaagcagagaatatatatctcaagaatatctgaatacaagctcctggatttacgagcagcaaaactaagttaccaatgtgcgagccgaaaaaagcgagtgaaaaacgtgcgaatatgccaactaactaaagacatttggattacaagaaacccacgcattttggattataaacattgcgacaggcagaaaaacctaagaatttcttcaacggcgccagcatggagaacccaacgcagaatgtaaacgaaaccaaggtggatttgggccaggagaaggagaaggaggcgtcgcaggaggaggagcatgccaccgccgtcaaggagaccatcattgacattcccgccgcgtgctccacttcctccaactcctcgtcgtacgacaccgattgcagcacggcgagcagcacctgctgcacccgccaaggcgagcacatctacatgcaacgcgaggccatcccggccacgccacttccggagtcggaggatatcggcctgctgaagtacgtccaccgccagcactggccctggttcatcctagtgatctccatcattgagattgccatcttcgcctacgaccgctacacaatgcccgcccagaatttcgggctacccgttccgattccgtcggattcggtgctggtctatcggccggaccggcgtctgcaggtgtggcgcttctttagctacatgttcctgcacgccaactggttccacctgggcttcaatatcgtcatccagctgttcttcggcattcccctggaggtgatgcacggcacggccaggatcggcgtgatctacatggcgggcgtttttgccggatccctgggcaccagtgtcgtcgactcggaggtcttcctggtgggcgccagcggtggcgtctatgccctgttggccgcacatctggccaacatcacattgaactatgcgcacatgaagagcgcatccacgcaactcggatcagttgtcatctttgtctcctgcgatctgggctatgctctctacacccaatacttcgatggaagcgccttcgccaagggtccccaggtgtcgtacattgcccacctgacgggagccctggcaggactaacgatcggcttcctggtgctgaagaacttcggtcatcgggagtacgagcagctcatctggtggctagcgttgggcgtctactgtgccttcaccgtcttcgccatcgttttcaacctgatcaacacggtgaccgcccagctgatggaggagcagggtgaggtgattacccagcatctgttgcacgacctgggagtgtcctaagtgtgaggttcggagtcgtcagcatgctcgcagggattcggaatctgcttgagcttcaggagagatcgagagacagagagttggtggaaaagaaaagttcactcaacgatttagttcaaaactaattcgatattcgtttggcttttgcttttcgttagcattatctcgttatcgttaccgtttgcagttaaacgtttcagttgcgaaacatagtacacaaactcataaaaaaaaaaacaaatcaagagaaatacactggacaaaaaaaagagcgaggagtgaggagaacataaaccgaagccgaaacgtgtaaacaaatgttgtgatagaaccaaagactgaatttatttcgcgtgtaaaaaccaagtaaaaatcaagaggaaaatcaaagaggagaaacagaactaatcgcctctcgctatgatttaaatgaacccaattatccatgttttcaattaatggtttgtctgttttcttaaattatgtatttattggccgcaattactacgaatgaatcgaatcgaagcatcagcaaactgtatcaaattgtttatacatccataagcataatgtgctccgaattgtaggattagtgttataatttatatatatttaggtataactagccctcctaacaaattgtttcaaattgtaaatactattaagtcgcacactagtcaaacaacaacaacagcaacagcaaaaacaacaaaaaatgtatggaaaaccacagcaaagaaccattcaattcagatcaattaagcaaatcgagttaaattaaattaaattatactaaagtcacttaatgcgttacaaaatcgagcaaatatttatcgtaatccctacacacacacacacacacactcgaaagtattactaattatatttatttatggtagggcagcgagggtttattaattcgtcaattgagcgaactatttatttatttatttatttaataatttagtgaaattcacacaaacaagcacgaaaaaacaacaacaacacaagagagagaagaaacaaaccaattcaactgtaaaaatatccaattgaaaaatacacacgaaaagccaaagaaaataaaaaatcaaaacatttcaagaatacaacagtaataacaaaatacaaaaaa

SEQ ID NO: 72 - protein, 355 aa, Drosophila melanogaster
MENPTQNVNETKVDLGQEKEKEASQEEEHATAVKETIIDIPAACSTSSNSSSYDTDCSTASSTCCTRQGEHIYMQREAIPATTLPESEDIGLLKYVHRQHWPWFILVISIIEIAIFAYDRYTMPAQNFGLPVPIPSDSVLVYRPDRRLQVWRFFSYMFLHANWFHLGFNIVIQLFFGIPLEVMHGTARIGVIYMAGVFAGSLGTSVVDSEVFLVGASGGVYALLAAHLANITLNYAHMKSASTQLGSVVIFVSCDLGYALYTQYFDGSAFAKGPQVSYIAHLTGALAGLTIGFLVLKNFGHREYEQLIWWLALGVYCAFTVFAIVFNLINTVTAQLMEEQGEVITQHLLHDLGVS

SEQ ID NO: 73 - DNA, 1121 bp, Drosophila melanogaster
ccagaaagcaaaatagaaacaaattttccatatttcatgctaaattggcacagatccgtactactatgctcatgagtcgagcgctttgccggagctggctaccccaggtggcccgcagatgtcatgctaatgtgaatgtgccaatcctgcggataaactctggtcatccggcggcgaggtcatgtcggcagattcacagcaaccgaaaacagagcagcaacctgaagccgacgactggggagcctgcggcagcggagcagaacaccccggtgccggtgaacaatgtgatcaaggcggtggccttcacgggagcatttacggtcggctgctttgcgggtgccaccatcctggagtacgagaacacacgtagcctaatcctagaaaaggctcgccaggcgagattcggttggtggcagagtcgttcgctggcggacagggattactggacacagatcaaacaagacatccggcggcactgggactcactgacacccggcgacaagatgtttgctcctatcttactctgcaatttggtggccttcgccatgtggcgggtgcccgctctgaaatccacaatgattacctacttcacatccaatccagcggcgaaagtcgtctgctggcccatgttcctgtccacattcagccattactcggctatgcaccttttcgccaatatgtacgtgatgcacagctttgccaacgctgcggctgtatcgttgggtaaagagcaattcttagcggtctacctcagcgccggcgtcttctccagtctgatgagcgtgctctacaaggcggccacgagtcaggcggggatgtccctgggtgcgtctggagctataatgacactgctggcctatgtatgcacccagtatccggacacacaacttagcattctctttctacccgcgttgacattctccgctggagctggtattaaagtgctaatgggcatcgactttgctggcgtcgtgatgggctggaagttcttcgatcacgcagcgcatttgggcggcgccatgtttggcatcttttgggccacgtatggggcacagatatgggcaaagcgcattggtctactgaattactaccatgacctgcgccggacgaagcagaaatag

SEQ ID NO: 74 - protein, 351 aa, Drosophila melanogaster
MLMSRALCRSWLPQVARRCHANVNVPILRINSGHPAARSCRQIHSNRKQSSNLKPTTGEPAAAEQNTPVPVNNVIKAVAFTGAFTVGCFAGATILEYENTRSLILEKARQARFGWWQSRSLADRDYWTQIKQDIRRHWDSLTPGDKMFAPILLCNLVAFAMWRVPALKSTMITYFTSNPAAKVVCWPMFLSTFSHYSAMHLFANMYVMHSFANAAAVSLGKEQFLAVYLSAGVFSSLMSVLYKAATSQAGMSLGASGAIMTLLAYVCTQYPDTQLSILFLPALTFSAGAGIKVLMGIDFAGVVMGWKFFDHAAHLGGAMFGIFWATYGAQIWAKRIGLLNYYHDLRRTKQK

(19) United States
(12) Patent Application Publication
(10) Pub. No.: US 2005/0095604 A1
(43) Pub. Date: May 5, 2005

(54) Novel modulators of amyloid-beta production and uses thereof

(76) Inventors: Tae-Wan Kim, Old Tappan, NJ (US); Hahn-Jun Lee, Fort Lee, NJ (US)

Correspondence Address: Brown Raysman Millstein Felder & Steiner LLP, 163 Madison Avenue, P.O. Box 1989, Morristown, NJ, 07962-1989, US

(21) Appl. No.: 10/702,578

(22) Filed: Nov. 5, 2003

(51) Int. Cl. G01N 33/00; C12Q 1/68; C07H 21/04; C07K 14/47

(52) U.S. Cl. 435/6; 435/69.1; 435/320.1; 435/325; 530/350; 536/23.5

(57) ABSTRACT

The present invention provides isolated nucleic acid sequences encoding presenilin stabilization factor (PSF) and PSF-like (PSFL) protein, vectors comprising same, host cells transformed with the vectors, and transgenic animals containing the host cells. The present invention further provides purified PSF and PSFL polypeptides, methods for making same, and pharmaceutical compositions comprising the polypeptides. Also provided are agents reactive with the nucleic acid sequences and polypeptides, kits comprising same, and methods for producing same. The present invention further provides methods for decreasing amyloid-beta (Aβ) production, destabilizing presenilin or nicastrin, destabilizing a gamma-secretase complex, and inhibiting activity of gamma-secretase, and pharmaceutical compositions for accomplishing same. The present invention further provides methods for treating neurodegeneration in a subject. Finally, the present invention provides an in vitro system for identifying an agent that modulates production of Aβ or an Aβ precursor, methods for making and using same, and agents identified by same.

### STATEMENT OF GOVERNMENT INTEREST

This invention was made with government support under NIH-NIA Grant No. AG18026. As such, the United States government has certain rights in this invention.

### BACKGROUND OF THE INVENTION

Alzheimer's disease is a neurodegenerative disease characterized by a progressive, inexorable loss of cognitive function (Francis et al., Neuregulins and ErbB receptors in cultured neonatal astrocytes. J. Neurosci. Res., 57:487-94, 1999) that eventually leads to an inability to maintain normal social and/or occupational performance. Alzheimer's disease is the most common form of age-related dementia, and one of the most serious health problems, in the United States. Approximately 4 million Americans suffer from Alzheimer's disease, at an annual cost of at least $100 billion —making Alzheimer's disease the third most costly disorder of aging. Alzheimer's disease is about twice as common in women as in men, and accounts for more than 65% of the dementias in the elderly. Alzheimer's disease is the fourth leading cause of death in the United States. To date, a cure for Alzheimer's disease is not available, and cognitive decline is inevitable.

The pathogenesis of Alzheimer's disease is associated with an excessive amount of neurofibrillary tangles (composed of paired helical filaments and tau proteins) and neuritic or senile plaques (composed of neurites, astrocytes, and glial cells around an amyloid core) in the cerebral cortex. While senile plaques and neurofibrillary tangles occur with normal aging, they are much more prevalent in persons with Alzheimer's disease. Specific protein abnormalities also occur in Alzheimer's disease. In particular, it is thought that amyloid-beta (AP) protein contributes to the pathogenesis of the disease. Thus, ongoing research into the production of amyloid-beta protein in Alzheimer's disease is needed.

Most cases of early-onset familial Alzheimer's disease (FAD) are caused by mutations in two related genes encoding presenilin proteins: PS1 and PS2 (Tanzi et al., The gene defects responsible for familial Alzheimer's disease. Neurobiol. Dis., 3:159-68, 1996; Hardy, J., Amyloid, the presenilins and Alzheimer's disease. Trends Neurosci., 20:154-59, 1997; Selkoe, D. J., Alzheimer's disease: genes, proteins, and therapy. Physiol. Rev., 81:741-66, 2001). FAD-associated mutations in the presenilins give rise to an incrased production of a longer (42 amino acid residues), more amyloidogenic form of amyloid-beta (Aβ42). Deciphering the pathobiology associated with the presenilins provides a unique opportunity to elucidate a molecular basis for Alzheimer's disease.

Presenilins are required for intramembrane proteolysis of selected type-I membrane proteins, including amyloid-beta precursor protein (APP), to yield amyloid-beta protein (De Strooper et al., Deficiency of presenilin-1 inhibits the normal cleavage of amyloid precursor protein. Nature, 391:387-90, 1998; Steiner and Haass, Intramembrane proteolysis by presenilins. Nat. Rev. Mol. Cell. Biol., 1:217-24, 2000; Ebinu and Yankner, A rip tide in neuronal signal transduction. Neuron, 34:499-502, 2002; De Strooper and Annaert, Presenilins and the intramembrane proteolysis of proteins: facts and fiction. Nat. Cell Biol., 3:E221-25, 2001; Sisodia and George-Hyslop, γ-Secretase, Notch, α-beta and Alzheimer's disease: where do the presenilins fit in? Nat. Rev. Neurosci., 3:281-90, 2002). Such proteolysis may be mediated by presenilin-dependent γ-secretase machinery, which is known to be highly conserved across species, including nematodes, flies, and mammals (L'Hemault and Arduengo, Mutation of a putative sperm membrane protein in Caenorhabditis elegans prevents sperm differentiation but not its associated meiotic divisions. J. Cell. Biol., 119:55-58, 1992; Levitan and Greenwald, Facilitation of lin-12-mediated signalling by sel-12, a Caenorhabditis elegans S182 Alzheimer's disease gene. Nature, 377:351-54, 1999; Li and Greenwald, HOP-1, a Caenorhabditis elegans presenilin, appears to be functionally redundant with SEL-12 presenilin and to facilitate LIN-12 and GLP-1 signaling. Proc. Natl. Acad. Sci. USA, 94:12204-209, 1997; Steiner and Haass, Intramembrane proteolysis by presenilins. Nat. Rev. Mol. Cell. Biol., 1:217-24, 2000; Sisodia and George-Hyslop, γ-Secretase, Notch, α-beta and Alzheimer's disease: where do the presenilins fit in? Nat. Rev. Neurosci., 3:281-90, 2002).

γ-Secretase mediates the final step in amyloid-β-protein (Aβ) production in Alzheimer's disease. Recent biochemical evidence has indicated that γ-secretase is a high-molecular-weight, multi-protein complex harboring presenilin heterodimers (Li et al., Presenilin 1 is linked with γ-secretase activity in the detergent solubilized state. Proc. Natl. Acad. Sci. USA, 97:613843, 2000; Esler et al., Activity-dependent isolation of the presenilin-γ-secretase complex reveals nicastrin and a gamma substrate. Proc. Natl. Acad. Sci. USA, 99:2720-25, 2002) and nicastrin. The stabilization of presenilin heterodimers (converted from a short-lived pool to a long-lived pool) and other undefined core components appears to be critical for γ-secretase activity (Thinakaran et al., Evidence that levels of presenilins (PS1 and PS2) are coordinately regulated by competition for limiting cellular factors. J. Biol. Chem., 272:28415-422, 1997; Tomita et al., The first proline of PALP motif at the C terminus of presenilins is obligatory for stabilization, complex formation, and gamma-secretase activities of presenilins. J. Biol. Chem., 276:33273-281, 2001). Genetic studies have also demonstrated that, in addition to presenilin, nicastrin is required for the transmembrane cleavage of Notch in Drosophila (Chung and Struhl, Nicastrin is required for Presenilin-mediated transmembrane cleavage in Drosophila. Nature Cell Biol., 3:1129-32, 2001; Hu and Fortini, Nicastrin is required for gamma-secretase cleavage of the Drosophila Notch receptor. Dev. Cell, 2:69-78, 2002; Lopez-Schier and St. Johnston, Drosophila nicastrin is essential for the intramembranous cleavage of notch. Dev. Cell, 2:79-89, 2002). However, prior to the present invention, it was not known precisely which molecules contribute to the stabilization of presenilin, the stabilization of nicastrin, the stabilization of the γ-secretase complex, and the modulation of activity of the γ-secretase complex, nor was it known how such molecules may interact to contribute to the production of amyloid-beta in Alzheimer's disease.

### SUMMARY OF THE INVENTION

Using an assay system based on RNA interference (RNAi), the inventors have determined that the suppression of Drosophila or human forms of PSF (presenilin stabilization factor)—homologues of nematode APH-1—abrogates the γ-secretase-mediated generation of Aβ, and also disrupts the stability of both presenilin and nicastrin. Furthermore, using affinity isolation experiments, the inventors have demonstrated that PSF forms a complex with nicastrin and presenilin 1. Thus, as shown herein, PSF is required for γ-secretase activity, and for the stabilization of presenilin and nicastrin. These findings suggest a critical role for PSF in the formation of a functional γ-secretase complex, and, thus, in the production of amyloid-beta production in Alzheimer's disease.

Accordingly, the present invention provides an isolated nucleic acid sequence encoding a polypeptide, wherein the polypeptide is selected from the group consisting of presenilin stabilization factor (PSF) and PSF-like protein (PSFL). In one embodiment of the invention, the nucleic acid has a nucleotide sequence selected from the group consisting of SEQ ID NOs: 1, 2, 3, 7, 9, 11, 13, 15, 17, 18, and 20. Also provided is an isolated nucleic acid sequence that hybridizes under high-stringency conditions to a second nucleic acid sequence, wherein the second nucleic acid sequence is complementary to a nucleotide sequence selected from the group consisting of SEQ ID NOs: 1, 2, 3, 7, 9, 11, 13, 15, 17, 18, and 20, or to a continuous fragment thereof.

The present invention further provides a purified polypeptide, selected from the group consisting of PSF and PSFL. In one embodiment of the invention, the polypeptide has an amino acid sequence selected from the group consisting of SEQ ID NOs: 4, 5, 6, 8, 10, 12, 14, 16, 19, 21, and 70. Also provided is a purified polypeptide encoded by a nucleic acid sequence that hybridizes under high-stringency conditions to a second nucleic acid sequence, wherein the second nucleic acid sequence is complementary to a nucleotide sequence selected from the group consisting of SEQ ID NOs: 1, 2, 3, 7, 9, 11, 13, 15, 17, 18, and 20, or to a continuous fragment thereof.

Additionally, the present invention provides a pharmaceutical composition, comprising a pharmaceutically acceptable carrier and PSF or PSFL.

The present invention is also directed to an antibody specific for a polypeptide, wherein the polypeptide is selected from the group consisting of PSF and PSFL.

The present invention further provides a method for producing an antibody specific for a polypeptide selected from the group consisting of PSF and PSFL, by: (a) immunizing a mammal with the selected polypeptide; and (b) purifying antibody from a tissue of the mammal or from a hybridoma made using tissue of the mammal. Also provided is an antibody produced by this method.

The present invention also provides a kit for use in detecting expression of PSF and/or PSFL, comprising: (a) an agent reactive with PSF and/or PSFL protein or PSF and/or PSFL nucleic acid; and (b) reagents suitable for detecting expression of PSF and/or PSFL.

The present invention further provides a vector comprising a nucleic acid sequence encoding a polypeptide, wherein the polypeptide is selected from the group consisting of PSF and PSFL. Also provided are a host cell transformed with the vector and a transgenic animal containing the host cell.

In addition, the present invention provides a method for making a polypeptide selected from the group consisting of PSF and PSFL, by: (a) introducing into a host cell a nucleic acid sequence encoding the selected polypeptide; (b) maintaining the host cell under conditions such that the nucleic acid sequence is expressed to produce the selected polypeptide; and (c) recovering the selected polypeptide.

The present invention further provides a method for decreasing amyloid-beta production in a cell, by decreasing activity of a presenilin-stabilizing molecule in the cell. In one embodiment of the invention, the molecule is PSF or PSFL. Also provided is a pharmaceutical composition for decreasing amyloid-beta production, comprising a pharmaceutically acceptable carrier and an inhibitor of a presenilin-stabilizing molecule. In one embodiment of the invention, the molecule is PSF or PSFL.

The present invention is also directed to a method for destabilizing presenilin or nicastrin in a cell, by decreasing activity of a presenilin-stabilizing molecule in the cell. In one embodiment of the invention, the molecule is PSF or PSFL.

The present invention also provides a method for destabilizing a gamma-secretase complex in a cell, by decreasing activity of a presenilin-stabilizing molecule in the cell. In one embodiment of the invention, the molecule is PSF or PSFL.

The present invention further provides a method for inhibiting activity of gamma-secretase in a cell, by decreasing activity of a presenilin-stabilizing molecule in the cell. In one embodiment of the invention, the molecule is PSF or PSFL.

The present invention is also directed to a method for decreasing amyloid-beta production in a cell, by increasing activity of a rhomboid peptide in the cell. In one embodiment of the invention, the peptide is rhomboid 1 or rhomboid 7. Also provided is a pharmaceutical composition for decreasing amyloid-beta production, comprising a rhomboid peptide, or a modulator of the peptide's expression, and a pharmaceutically acceptable carrier. In one embodiment of the invention, the peptide is rhomboid 1 or rhomboid 7.

The present invention further provides a method for treating neurodegeneration in a subject in need of treatment, by administering to the subject an inhibitor of a presenilin-stabilizing molecule, in an amount effective to treat the neurodegeneration. In one embodiment of the invention, the molecule is PSF or PSFL.

The present invention is also directed to a method for treating neurodegeneration in a subject in need of treatment, by administering to the subject a rhomboid peptide, or a modulator of the peptide's expression, in an amount effective to treat the neurodegeneration. In one embodiment of the invention, the peptide is selected from the group consisting of rhomboid 1 and rhomboid 7.

Additionally, the present invention provides an in vitro system for identifying an agent that selectively modulates production of amyloid-beta or an amyloid-beta precursor, comprising Drosophila-derived S2 cells that express human APP, a human APP derivative, or a human presenilin.

The present invention further provides a method for making an in vitro system for identifying an agent that selectively modulates production of amyloid-beta or an amyloid-beta precursor, by generating Drosophila-derived S2 cells that express human APP, a human APP derivative, or a human presenilin. Also provided is an in vitro system made by this method.

The present invention is also directed to a method for identifying a protein product that modulates production of amyloid-beta or an amyloid-beta precursor, by: (a) obtaining or generating Drosophila-derived S2 cells that express human APP, a human APP derivative, or a human presenilin; (b) contacting the cells with dsRNA for a candidate protein product; and (c) assessing the ability of the dsRNA to modulate production of amyloid-beta or an amyloid-beta precursor in the cells, wherein ability of the dsRNA to modulate production of amyloid-beta or an amyloid-beta precursor is indicative that the candidate protein product modulates production of amyloid-beta or an amyloid-beta precursor. Also provided are a protein product identified by this method, and a method for treating neurodegeneration in a subject in need of treatment by administering this protein product to the subject in an amount effective to treat the neurodegeneration.

The present invention is further directed to a method for identifying an agent that modulates production of amyloid-beta or an amyloid-beta precursor, by: (a) obtaining or generating Drosophila-derived S2 cells that express human APP, a human APP derivative, or a human presenilin; (b) contacting the cells with a candidate agent; and (c) assessing the ability of the candidate agent to modulate production of amyloid-beta or an amyloid-beta precursor in the cells. Also provided are an agent identified by this method, and a method for treating neurodegeneration in a subject in need of treatment by administering this agent to the subject in an amount effective to treat the neurodegeneration.

Additional aspects of the present invention will be apparent in view of the description which follows.

### BRIEF DESCRIPTION OF THE FIGURES

FIG. 1 depicts stable expression of human APP C-terminal fragments (C99) and Drosophila presenilins (dPS) in Drosophila S2 cells. A: Schematic representation of Drosophila expression constructs: APP-C99 with a C-terminal HA-epitope tag under the control of the Actin promoter (pAc), and dPS harboring an HA-epitope tag in the large hydrophilic loop region under control of the Tubulin promoter (pTu). Lysates (B) or media (C) from stable S2 cell lines, expressing dPS, C99, or both (dPS+C99), were analyzed by Western blotting using anti-HA (B) or anti-Aβ antibody 6E10 (C). D: Accumulation of APP C-terminal fragments (C99/83) correlated with a decrease in Aβ generation after treatment with a γ-secretase inhibitor, Compound E (Cpd. E).

FIG. 2 shows that nicastrin modulates Aβ generation and presenilin stability. A: Effects of RNAi-mediated inhibition of Drosophila presenilins (dPS) (left panel) or Drosophila nicastrin (dNic) (right panel) on Aβ generation in S2 cells. Stable S2 cells expressing APP-C99 were treated for 3 days with increasing amounts of double-stranded RNA (dsRNA) against either dPS or dNic. The media were assayed by Western-blot analysis using 6E10. B: Effects of dNic downregulation, mediated by RNAi, on the stability of dPS. Double-stable S2 cells expressing dPS and APP-C99 were treated with dsRNA directed against dPS, dNic, or both (dPS+dNic). Lysates (top) and media (bottom) were analyzed by Western blotting using anti-HA or anti-Aβ antibodies, respectively. Arrows denote full-length dPS (dPS-FL), endoproteolytic dPS C-terminal fragments (dPS-CTF), human APP C-terminal fragments (C99/83), and secreted Aβ. C, D: Mammalian presenilins and nicastrin are interdependent for their stabilization. Effects of synthetic, small interference RNA (siRNA) directed against human nicastrin on the stability of PS1 or PS2, and vice versa, in human HEK293 cells. Cells were treated with indicated siRNA, and the levels of endogenous PS1, PS2, or nicastrin were determined by Western-blot analyses.

FIG. 3 illustrates that PSF modulates Aβ generation and stabilizes presenilin endoproteolytic fragments. A: dPSF RNAi inhibits Aβ generation. Stable S2 cells expressing APP-C99 were treated with dsRNA of Drosophila versions of PSF (dPSF, top panel), SKIP (middle panel), or β-catenin (bottom panel), for 3 days. The media were assayed by Western-blot analysis using 6E10. B: dPSF RNAi causes an increase in the accumulation of γ-secretase substrates, C99/83, and a decrease in the cellular levels of dPS CTF, but does not affect dPS-FL. Double-stable S2 cells co-expressing dPS and C99 were treated with the indicated amounts of dsRNA directed against dPSF. Lysates were analyzed by Western blotting using anti-HA antibody. C: Effects of dPSF RNAi, dNic RNAi, or dPS RNAi on the stability of dPS CTF and dPS FL. Stable S2 cells co-expressing dPS and dNic were treated with indicated dsRNA for 3 days, and lysates were analyzed by Western blotting.

FIG. 4 shows that PSF is required for nicastrin stability in Drosophila S2 and HEK293 cells. A: Effects of RNAi-mediated inhibition of PSF on nicastrin stability. Stable S2 cells expressing dNic (with a C-terminal V5-epitope tag) were treated with dsRNAs of dPSF, dNic, or dPS, and the lysates were analyzed by Western blotting. B (left and right panels): Human PSF modulates the stabilization of PS1, PS2, and nicastrin in mammalian cells. HEK293 cells were treated with siRNAs directed against human PSF, PSF-like protein (PSFL), or β-catenin, and lysates were analyzed with PS1, PS2, or nicastrin antibodies.

FIG. 5 depicts cloning and expression patterns of human PSF. A: cDNA of three PSF isoforms —PSF (also referred to herein as “PSF1”) (SEQ ID NO:1), PSFa (SEQ ID NO:2), and PSFb (SEQ ID NO:3), and translated-protein sequences of PSF1 (SEQ ID NO:4), PSFa (SEQ ID NO:5), and PSFb (SEQ ID NO:6). Translated amino acid sequences are located below each corresponding section of nucleic acid sequence, and predicted transmembrane domains are underlined. The human PSF gene is located on chromosome 1, and consists of 6 exons. The limits of individual exons for human PSF are noted by arrows, and individual exons are indicated by numbers, immediately below the protein sequences. The predicted signal peptide sequences (SP) are italicized, and PSFb-specific sequences are underlined and italicized. Insertion of a single “c” at position 727 of the PSF gene gives rise to a truncated polypeptide, PSFa, while the deletion of base pairs from position 735 through to position 1073 gives rise to a PSFb transcript encoding different C-terminal ends with 20 extra amino acids. B: Predicted topology of PSF with 6 transmembrane domains. Heterogeneous C-terminal ends of PSF (PSF1), PSFa, and PSFb are indicated by colored boxes. C: Expression patterns of PSF/PSFa (PSF1/PSFa) and PSFb in different human tissues. D: Expression patterns of PSF/PSFa (PSF1/PSFa) and PSFb in different regions of the brain.

FIG. 6 illustrates the characterization of PSF in transfected cells, and the co-purification of PSF with PS1 and nicastrin. A: Detection of hPSF in HEK293 cells. Lysates prepared from cells transiently transfected with vector alone or with V5-epitope-tagged PSF (PSF-V5) were analyzed by Western blotting using anti-V5 antibody. B-E: PSF co-purified with nicastrin and PS1. Lysates (containing 1% CHAPSO) prepared from stable HEK293 cells expressing hPSF (with V5 tag and 6×His; clonal line: 2-7) were subjected to incubation with metal affinity resins (Talon). The resulting PSF-bearing complex was then analyzed by Western blotting using antibodies to PS1 (B), nicastrin (C), PSF (D), or β-catenin (E). Vector-expressing cells (clonal line: 1-2) were used as controls for the affinity isolation procedure.

FIG. 7 depicts mass spectra of AP peptides resulting from IP/MS analysis, and an Aβ peptide profile from Drosophila S2 cells stably expressing human APP-C99. The Aβ number used reflects the true peptide length (Aβ1-38, Aβ1-40, and Aβ1-42). Note that Drosophila cells produce substantial levels of Aβ38 (whereas mammalian cells produce significantly less Aβ38), and Aβ42 is clearly detectable. Therefore, S2 cells expressing human APP-C99 can be used to screen small molecules that selectively modulate Aβ42 generation.

FIG. 8 sets forth the cDNA sequence (GenBank accession number AF508787) (SEQ ID NO:7) (A) and the amino acid sequence (SEQ ID NO:8) (B) for human PSF (PSF1).

FIG. 9 sets forth the cDNA sequence (GenBank accession number AY113698) (SEQ ID NO:9) (A) and the amino acid sequence (SEQ ID NO:10) (B) for human PSFa.

FIG. 10 sets forth the cDNA sequence (GenBank accession number AY113699) (SEQ ID NO:11) (A) and the amino acid sequence (SEQ ID NO:12) (B) for human PSFb.

FIG. 11 sets forth the cDNA sequence (GenBank accession number AF508794) (SEQ ID NO:13) (A) and the amino acid sequence (SEQ ID NO:14) (B) for human PSFL.

FIG. 12 sets forth the cDNA sequence (GenBank accession number AF508786) (SEQ ID NO:15) (A) and the amino acid sequence (SEQ ID NO:16) (B) for Drosophila PSF.

FIG. 13 sets forth the genomic sequence for human PSF (SEQ ID NO:17).

FIG. 14 sets forth the cDNA sequence (SEQ ID NO:18) (A) and the amino acid sequence (SEQ ID NO:19) (B) for mouse PSFa.

FIG. 15 sets forth the cDNA sequence (SEQ ID NO:20) (A) and the amino acid sequence (SEQ ID NO:21) (B) for mouse PSFb.

FIG. 16 sets forth the amino acid sequence for mouse PSFL (SEQ ID NO:70).

FIG. 17 sets forth the nucleotide sequence (SEQ ID NO:71) (A) and the amino acid sequence (SEQ ID NO:72) (B) for Drosophila rhomboid 1.

FIG. 18 sets forth the nucleotide sequence (SEQ ID NO:73) (A) and the amino acid sequence (SEQ ID NO:74) (B) for Drosophila rhomboid 7.

FIG. 19 illustrates that RNAi of Drosophila rhomboid 1 or rhomboid 7 potentiates amyloid-beta generation. Stable S2 cells expressing APP-C99 were treated with dsRNA directed against rhomboid 1 (top) or rhomboid 7 (bottom) for 3 days. The media were assayed by Western-blot analysis using anti-Aβ antibody, 6E10.

### DETAILED DESCRIPTION OF THE INVENTION

The present invention is directed to novel genes and proteins, including presenilin stabilization factor (PSF) and PSF-like protein (PSFL), that modulate the production of amyloid-beta protein (Aβ). As disclosed herein, the PSF gene is located on chromosome 1, 1p36.13-q31.3 (locus ID 51107), within the chromosome 1 risk locus for Alzheimer's disease. Thus, the coding and non-coding PSF genomic DNA sequence, along with microsatellite markers and SNP around or within the PSF locus, may be used to screen and identify other risk loci for Alzheimer's disease. Additionally, the human PSF gene encodes a polypeptide with six predicted transmembrane domains. As further disclosed herein, the PSF gene encodes at least three forms of PSF in the human population, namely, PSF1, PSFa, and PSFb.

In view of the foregoing, the present invention provides: a PSF gene; an isolated nucleic acid sequence encoding a PSF polypeptide; a PSFL gene; and an isolated nucleic acid sequence encoding a PSFL polypeptide. The PSF gene, and the nucleic acid sequence encoding PSF protein, include PSF1, PSFa, and PSFb. The PSF and PSFL genes may be “endogenous” genes, which are ones that originate or arise naturally, from within an organism, or “exogenous” genes, which originate or arise outside an organism. Due to the degeneracy of the genetic code, the PSF gene of the present invention includes a multitude of nucleic acid substitutions that will also encode a PSF polypeptide, including the PSF1, PSFa, and PSFb isoforms of the protein, and the PSFL gene of the present invention includes a multitude of nucleic acid substitutions that will also encode a PSFL polypeptide.

As used herein, a “PSF polypeptide” includes, where appropriate, both a PSF protein (including PSF1, PSFa, and PSFb isoforms) and a “PSF analogue”; and a “PSFL polypeptide” includes, where appropriate, both a PSFL protein and a “PSF analogue”. Unless otherwise indicated, “protein” shall mean a protein, protein domain, polypeptide, or peptide, and shall include any fragment thereof. A “PSF analogue” may be any protein having functional similarity to the PSF protein that is 60% or greater (preferably, 70% or greater) in amino-acid-sequence homology with the PSF protein. A “PSFL analogue” may be any protein having functional similarity to the PSFL protein that is 60% or greater (preferably, 70% or greater) in amino-acid-sequence homology with the PSFL protein.

The nucleic acid sequence of the present invention may be genomic DNA, cDNA, antisense DNA, mRNA, dsRNA, siRNA, single-stranded RNA (ssRNA), or antisense RNA, and may be derived from any species, including human, insect, and mouse. In one embodiment of the present invention, the nucleic acid sequence is derived from a mammalian species, preferably a human. In another embodiment of the present invention, the nucleic acid sequence is derived from an insect species, preferably Drosophila melanogaster.

Where the nucleic acid sequence of the present invention encodes PSF, the nucleic acid sequence may be PSF1, PSFa, or PSFb. In one embodiment of the present invention, the nucleic acid sequence of PSF1 comprises the nucleotide sequence of SEQ ID NO:1, SEQ ID NO:7 (GenBank accession number AF508787), SEQ ID NO:15, or SEQ ID NO:17 (including conservative substitutions thereof). “Conservative substitutions”, as used herein, are those amino acid substitutions which are functionally equivalent to the substituted amino acid residue, either because they have similar polarity or steric arrangement, or because they belong to the same class as the substituted residue (e.g., hydrophobic, acidic, or basic). In a preferred embodiment, the PSF1 nucleic acid sequence of the present invention comprises the nucleotide sequence of SEQ ID NO:17. In another embodiment of the present invention, the nucleic acid encodes a PSF1 protein having the amino acid sequence of SEQ ID NO:4, SEQ ID NO:8, or SEQ ID NO:16. In a preferred embodiment, the PSF1 nucleic acid sequence of the present invention encodes a human PSF1 protein having the amino acid sequence of SEQ ID NO:8.

Additionally, in one embodiment of the present invention, the nucleic acid sequence of PSFa comprises the nucleotide sequence of SEQ ID NO:2, SEQ ID NO:9 (GenBank accession number AY113698), or SEQ ID NO:18 (including conservative substitutions thereof). In a preferred embodiment, the PSFa nucleic acid sequence of the present invention comprises the nucleotide sequence of SEQ ID NO:9. In another embodiment of the present invention, the nucleic acid encodes a PSFa protein having the amino acid sequence of SEQ ID NO:10 or SEQ ID NO:19. In a preferred embodiment, the PSFa nucleic acid sequence of the present invention encodes a PSFa protein having the amino acid sequence of SEQ ID NO:10.

In a further embodiment of the present invention, the nucleic acid sequence of PSFb comprises the nucleotide sequence of SEQ ID NO:3, SEQ ID NO:11 (GenBank accession number AY113699), or SEQ ID NO:20 (including conservative substitutions thereof). In a preferred embodiment, the PSFb nucleic acid sequence of the present invention comprises the nucleotide sequence of SEQ ID NO:11. In another embodiment of the present invention, the PSFb nucleic acid encodes a PSFb protein having the amino acid sequence of SEQ ID NO:12 or SEQ ID NO:21. In a preferred embodiment, the PSFb nucleic acid sequence of the present invention encodes a PSFb protein having the amino acid sequence of SEQ ID NO:12.

Where the nucleic acid sequence of the present invention encodes PSFL, the nucleic acid sequence preferably comprises the nucleotide sequence of SEQ ID NO:13 (GenBank accession number AF508794) (including conservative substitutions thereof). In another embodiment of the present invention, the nucleic acid encodes a PSFL protein having the amino acid sequence of SEQ ID NO:14 or SEQ ID NO:70. In a preferred embodiment, the PSFL nucleic acid sequence of the present invention encodes a PSFL protein having the amino acid sequence of SEQ ID NO:14.

The present invention also provides an isolated nucleic acid sequence that hybridizes, preferably under high-stringency conditions (e.g., hybridization to filter-bound DNA in 0.5-M NaHPO₄ at 65° C. and washing in 0.1×SSC/0.1% SDS at 68° C.) or moderate-stringency conditions (e.g., washing in 0.2×SSC/0.1% SDS at 42° C.) (Ausubel et al., Current Protocols in Molecular Biology (New York: John Wiley and Sons, New York, 1997)), to a second nucleic acid that is complementary to the nucleotide sequence of SEQ ID NO:1, SEQ ID NO:2, SEQ ID NO:3, SEQ ID NO:7, SEQ ID NO:9, SEQ ID NO:11, SEQ ID NO:13, SEQ ID NO:15, SEQ ID NO:17, SEQ ID NO:18, or SEQ ID NO:20, or to a continuous fragment thereof. In addition, the present invention provides a nucleic acid sequence, encoding presenilin stabilization factor (PSF) protein (including PSF1, PSFa, and PSFb) or PSF-like protein (PSFL), that has one or more mutations, wherein the mutations result in the expression of either a non-functional or mutant protein, or in a lack of expression altogether. The mutations may be generated by at least one of the following methods: point mutation, insertion mutation, rearrangement, or deletion mutation, or a combination thereof.

The present invention further provides isolated and purified polypeptides, including presenilin stabilization factor (PSF) protein (including the PSF1, PSFa, and PSFb isoforms) and PSF-like protein (PSFL). The PSF and PSFL polypeptides may be isolated from tissue (e.g., brain tissue) obtained from a subject, or recombinantly produced as described below. In one embodiment of the present invention, the polypeptide is derived from a mammalian species, preferably a human. In another embodiment of the present invention, the polypeptide is derived from an insect species, preferably Drosophila melanogaster.

In one embodiment of the present invention, the PSF1 isoform of the PSF polypeptide has the amino acid sequence of SEQ ID NO:4, SEQ ID NO:8, or SEQ ID NO:16. In a preferred embodiment, the PSF1 isoform of the present invention is human PSF1, and comprises the amino acid sequence of SEQ ID NO:8. In another embodiment of the present invention, the PSF1 isoform of the PSF polypeptide is encoded by the nucleotide sequence of SEQ ID NO:1, SEQ ID NO:7 (GenBank accession number AF508787), SEQ ID NO:15, or SEQ ID NO:17 (including conservative substitutions thereof). In a preferred embodiment, the PSF1 isoform of the present invention is encoded by the nucleotide sequence of SEQ ID NO:17.

Additionally, in one embodiment of the present invention, the PSFa isoform of the PSF polypeptide has the amino acid sequence of SEQ ID NO:5, SEQ ID NO:10, or SEQ ID NO:19. In a preferred embodiment, the PSFa isoform of the present invention is human PSFa, and comprises the amino acid sequence of SEQ ID NO:10. In another embodiment of the present invention, the PSFa isoform of the PSF polypeptide is encoded by the nucleotide sequence of SEQ ID NO:2, SEQ ID NO:9 (GenBank accession number AY113698), or SEQ ID NO:18 (including conservative substitutions thereof). In a preferred embodiment, the PSFa isoform of the present invention is encoded by the nucleotide sequence of SEQ ID NO:9.

In a further embodiment of the present invention, the PSFb isoform of the PSF polypeptide has the amino acid sequence of SEQ ID NO:6, SEQ ID NO:12, or SEQ ID NO:21. In a preferred embodiment, the PSFb isoform of the present invention is human PSFb, and comprises the amino acid sequence of SEQ ID NO:12. In another embodiment of the present invention, the PSFb isoform of the PSF polypeptide is encoded by the nucleotide sequence of SEQ ID NO:3, SEQ ID NO:11 (GenBank accession number AY 113699), or SEQ ID NO:20 (including conservative substitutions thereof). In a preferred embodiment, the PSFb isoform of the present invention is encoded by the nucleotide sequence of SEQ ID NO:11.

Where the polypeptide of the present invention is PSFL protein, the polypeptide preferably comprises the amino acid sequence of SEQ ID NO:14 or SEQ ID NO:70. In a preferred embodiment, the PSFL polypeptide of the present invention has the amino acid sequence of SEQ ID NO:14. In another embodiment of the present invention, the PSFL polypeptide is encoded by the nucleotide sequence of SEQ ID NO:13 (GenBank accession number AF508794) (including conservative substitutions thereof).

The present invention is further directed to a purified protein encoded by a nucleic acid sequence that hybridizes under high- or moderate-stringency conditions to a second nucleic acid sequence that is complementary to the nucleotide sequence of SEQ ID NO:1, SEQ ID NO:2, SEQ ID NO:3, SEQ ID NO:7, SEQ ID NO:9, SEQ ID NO:11, SEQ ID NO:13, SEQ ID NO:15, SEQ ID NO:17, SEQ ID NO:18, or SEQ ID NO:20, or to a continuous fragment thereof.

The present invention also provides a pharmaceutical composition comprising PSF protein (including the PSF1, PSFa, and PSFb isoforms) and a pharmaceutically-acceptable carrier, PSF nucleic acid (including PSF1, PSFa, and PSFb) and a pharmaceutically-acceptable carrier, PSFL protein and a pharmaceutically-acceptable carrier, or PSFL nucleic acid and a pharmaceutically-acceptable carrier. The pharmaceutically-acceptable carrier must be “acceptable” in the sense of being compatible with the other ingredients of the composition, and not deleterious to the recipient thereof. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc, and water, among others. Formulations of the pharmaceutical composition may be conveniently presented in unit dosage.

The formulations of the present invention may be prepared by methods well-known in the pharmaceutical arts. For example, the PSF protein, PSF nucleic acid, PSFL protein, or PSFL nucleic acid may be brought into association with a carrier or diluent, as a suspension or solution. Optionally, one or more accessory ingredients (e.g., buffers, flavoring agents, surface active agents, and the like) also may be added. The choice of carrier will depend upon the route of administration of the composition. The pharmaceutical composition may be useful for administering the PSF protein or nucleic acid, or the PSFL protein or nucleic acid, of the present invention to a subject to treat a variety of disorders. The PSF or PSFL protein or nucleic acid is provided in an amount that is effective to treat the disorder in a subject to whom the pharmaceutical composition is administered. This amount may be readily determined by the skilled artisan.

Additionally, the present invention provides agents that are reactive with a PSF protein (including the PSF 1, PSFa, and PSFb isoforms of the PSF protein) or a PSFL protein. As used herein, “reactive” means the agent has affinity for, binds to, or is directed against the PSF or PSFL protein. The agent that binds to, or reacts with, the PSF or PSFL protein may be either natural or synthetic. Furthermore, the agent may include, without limitation, an antibody, a compound, a drug, a Fab fragment, a F(ab′)₂ fragment, a molecule, a nucleic acid, a protein (including a growth factor), a polypeptide, a peptide, a nucleic acid (including genomic DNA, cDNA, antisense DNA, mRNA, dsRNA, siRNA, ssRNA, or antisense RNA), and any combinations thereof. A Fab fragment is a univalent antigen-binding fragment of an antibody, which is produced by papain digestion. A F(ab′)₂ fragment is a divalent antigen-binding fragment of an antibody, which is produced by pepsin digestion. Agents that bind to the PSF or PSFL protein may be identified or screened by contacting the protein with the agent of interest, and assessing the ability of the agent to bind to the protein.

In one embodiment of the invention, the agent is an antibody specific for, or immunoreactive with, PSF protein (including the PSF 1, PSFa, and PSFb isoforms) or PSFL protein. Preferably, the agent is an antibody specific for human PSF. The antibody of the present invention may be monoclonal or polyclonal, and may be produced by techniques well known to those skilled in the art. The antibody of the present invention may be incorporated into kits which include an appropriate labeling system, buffers, and other necessary reagents for use in a variety of detection and diagnostic applications. Labeling of the antibody of the present invention may be accomplished by standard techniques using one of the variety of different chemiluminescent and radioactive labels known in the art.

The present invention further provides a method for producing an antibody specific for the PSF polypeptide (including the PSF1, PSFa, and PSFb isoforms of the PSF protein) or the PSFL polypeptide, comprising the steps of: (a) immunizing a mammal with the selected polypeptide (e.g., PSF1, PSFa, PSFb, or PSFL); and (b) purifying antibody from a tissue of the mammal or from a hybridoma made using tissue of the mammal. For example, a polyclonal antibody may be produced by immunizing a rabbit, mouse, or rat with purified PSF or PSFL protein. Thereafter, a monoclonal antibody may be produced by removing the spleen from the immunized rabbit, mouse, or rat, and fusing the spleen cells with myeloma cells to form a hybridoma which, when grown in culture, will produce a monoclonal antibody. In a preferred embodiment of the present invention, the polypeptide is human PSF. The present invention also provides an antibody produced by the above-described method.

The present invention is further directed to agents that are reactive with a nucleic acid encoding a PSF (including PSF1, PSFa, and PSFb) or PSFL protein. Suitable agents include, but are not limited to, an antibody, a compound, a drug, a Fab fragment, a F(ab′)₂ fragment, a molecule, a nucleic acid, a protein, a polypeptide, a peptide, a nucleic acid (including genomic DNA, cDNA, antisense DNA, mRNA, dsRNA, siRNA, ssRNA, or antisense RNA), and any combinations thereof. In a preferred embodiment of the present invention, the agent is dsRNA that is directed against nucleic acid encoding PSF or PSFL protein, and which may be useful in RNAi interference. The agents that are reactive with the nucleic acid encoding PSF or PSFL may inhibit or promote expression of the nucleic acid. Such agents may be discovered by a method for screening for an agent that is reactive with a nucleic acid encoding PSF or PSFL, wherein the method comprises contacting the selected nucleic acid with an agent of interest, and assessing the ability of the agent to bind to the selected nucleic acid. An agent that inhibits or promotes the expression of a nucleic acid encoding PSF or PSFL may be screened by contacting the agent with a host cell transformed with a vector comprising the selected nucleic acid, and assessing the agent's effect on expression of the nucleic acid.

The present invention also provides nucleic acid probes and mixtures thereof that hybridize to nucleic acid encoding PSF (including PSF1, PSFa, and PSFb) or PSFL protein. Such probes may be prepared by a variety of techniques known to those skilled in the art, including, without limitation, PCR and restriction-enzyme digestion of nucleic acid encoding PSF or PSFL; and automated synthesis of oligonucleotides whose sequences correspond to selected portions of the nucleotide sequence of nucleic acid encoding PSF or PSFL, using commercially-available oligonucleotide synthesizers such as the Applied Biosystems Model 392 DNA/RNA synthesizer. The nucleic acid probes of the present invention also may be prepared so that they contain at least one point, insertion, rearrangement, or deletion mutation, or a combination thereof, to correspond to mutations of the PSF or PSFL gene.

The nucleic acid probes of the present invention may be DNA or RNA, and may vary in length from about 8 nucleotides to the entire length of the nucleic acid encoding PSF (including PSF1, PSFa, and PSFb) or PSFL. Preferably, the probes are 8 to 30 nucleotides in length. Labeling of the nucleic acid probes may be accomplished using one of a number of methods known in the art, including, without limitation, PCR, nick translation, end labeling, fill-in end labeling, polynucleotide kinase exchange reaction, random priming, or SP6 polymerase (for riboprobe preparation), and one of a variety of labels, including, without limitation, radioactive labels such as ¹⁵S, ³²P, or ³H and nonradioactive labels such as biotin, fluorescein (FITC), acridine, cholesterol, or carboxy-X-rhodamine (ROX). Combinations of two or more nucleic probes, corresponding to different or overlapping regions of nucleic acid encoding PSF or PSFL, also may be included in kits, for use in a variety of detection and diagnostic applications.

The discovery that the PSF gene is located on chromosome 1, 1 p36.13-q31.3 (locus ID 51107), within the chromosome 1 risk locus for Alzheimer's disease, suggests that there may be a correlation between expression of PSF (or PSFL) in a subject and the subject's risk of developing Alzheimer's disease. Such a correlation would provide a means of identifying patients who have Alzheimer's disease or another neurodegenerative disease, or who are at increased risk of developing such a disease. Such a correlation would also present the potential for commercial application in the form of a test to screen for susceptibility to the development of neurodegeneration, including Alzheimer's disease. The development of such a test could provide general screening procedures that may assist in the early detection and diagnosis of neurodegeneration, and/or provide a method for the follow-up of patients who have been diagnosed with neurodegeneration or who have been identified as being at increased risk of developing neurodegeneration.

Accordingly, the present invention further provides a kit for use in detecting expression of PSF and/or PSFL, comprising: (a) an agent reactive with PSF and/or PSFL; and (b) reagents suitable for detecting expression of PSF and/or PSFL. The kit of the present invention comprises at least one reagent for use in an assay to detect the presence of PSF protein (including the PSF 1, PSFa, and PSFb isoforms of the PSF protein) and/or PSFL protein, or at least one reagent for use in an assay to detect directly the presence of a nucleic acid sequence encoding PSF (including PSF1, PSFa, and PSFb) and/or PSFL. The kit of the present invention also may comprise instructions for assaying a diagnostic sample of a subject for the presence or expression of PSF and/or PSFL, and for using the kit to determine whether a subject has neurodegeneration (e.g., Alzheimer's disease), had neurodegeneration (in the case of autopsy), or is at increased risk of developing neurodegeneration. In one embodiment of the present invention, the kit further comprises a container in which the reagent and the instructions are packaged.

A kit designed to detect the presence or expression of PSF protein (including the PSF I, PSFa, and PSFb isoforms of the PSF protein) and/or PSFL protein may contain an agent specifically reactive with PSF and/or PSFL. The agent may be any of those described above, including an antibody (e.g., an allele-specific antibody) that selectively binds the PSF and/or PSFL protein, and may be used in any of the above-described assays or methods for detecting or quantifying the presence of PSF and/or PSFL. The kit of the present invention also may include at least one antibody directed to PSF and/or PSFL, preferably labeled with a detectable marker, along with a solid support capable of binding PSF and/or PSFL protein.

A kit designed to detect the presence of a nucleic acid sequence encoding PSF (including PSF1, PSFa, and PSFb) and/or PSFL may contain an agent specifically reactive with PSF and/or PSFL. The agent may be any of those described above, including oligonucleotide probes that selectively bind to a nucleic acid sequence encoding the PSF and/or PSFL gene, and may be used in any of the above-described assays or methods for detecting or quantifying the presence of one or more alleles of PSF and/or PSFL. The kit of the present invention also may include probes (e.g., allele-specific probes) that can hybridize to amplified fragments of a nucleic acid sequence corresponding to part or all of the PSF and/or PSFL gene, and that can be used to identify the presence of PSF and/or PSFL. Furthermore, a kit designed to detect the presence of a nucleic acid sequence encoding PSF and/or PSFL may contain primers which hybridize to a nucleic acid sequence corresponding to part or all of the PSF and/or PSFL gene, and which permit the amplification of this nucleic acid sequence (e.g., by LCR, PCR, and other amplification procedures known in the art).

The present invention is further directed to a vector comprising a nucleic acid sequence encoding PSF or PSFL polypeptide. The nucleic acid sequence encoding PSF polypeptide may be, for example, the PSF1, PSFa, or PSFb nucleotide sequence. In one embodiment of the present invention, the vector contains the nucleic acid sequence of PSF I, which comprises the nucleotide sequence of SEQ ID NO:1, SEQ ID NO:7 (GenBank accession number AF508787), SEQ ID NO:15, or SEQ ID NO:17 (including conservative substitutions thereof), or to a continuous fragment thereof. In another embodiment of the present invention, the vector contains the nucleic acid sequence of PSFa, which comprises the nucleotide sequence of SEQ ID NO:2, SEQ ID NO:9 (GenBank accession number AY113698), or SEQ ID NO:18 (including conservative substitutions thereof), or to a continuous fragment thereof. In a further embodiment of the present invention, the vector contains the nucleic acid sequence of PSFb, comprising the nucleotide sequence of SEQ ID NO:3, SEQ ID NO:11 (GenBank accession number AY113699), or SEQ ID NO:20 (including conservative substitutions thereof), or to a continuous fragment thereof. In yet another embodiment of the present invention, the vector contains the nucleic acid sequence of the PSFL gene, comprising the nucleotide sequence of SEQ ID NO:13 (GenBank accession number AF508794) (including conservative substitutions thereof), or to a continuous fragment thereof.

Alternatively, the nucleic acid sequence of the vector may comprise a nucleic acid sequence that hybridizes under high- or moderate-stringency conditions to the nucleotide sequence of SEQ ID NO:1, SEQ ID NO:2, SEQ ID NO:3, SEQ ID NO:7, SEQ ID NO:9, SEQ ID NO:11, SEQ ID NO:13, SEQ ID NO:15, SEQ ID NO:17, SEQ ID NO:18, or SEQ ID NO:20, or to a continuous fragment thereof. In a preferred embodiment of the present invention, the vector contains a nucleic acid sequence encoding human PSF.

The vector of the present invention may be constructed by inserting nucleic acid encoding PSF (e.g., PSF1, PSFa, or PSFb) or PSFL polypeptide into a suitable vector nucleic acid operably linked to an expression control sequence. The term “inserted”, as used herein, means the ligation of a foreign DNA fragment with vector DNA, by techniques such as the annealing of compatible cohesive ends generated by restriction endonuclease digestion, or by the use of blunt-end ligation techniques. Other methods of ligating DNA molecules will be apparent to one skilled in the art.

The vector of the present invention may be derived from a number of different sources, including plasmids, viral-derived nucleic acids, cosmids, lytic bacteriophage derived from phage lambda, and filamentous single-stranded bacteriophages such as M13. Depending upon the type of host cell into which the vector is introduced, vectors may be bacterial or eukaryotic. Bacterial vectors are derived from many sources, including the genomes of plasmids and phages. Eukaryotic vectors are constructed from a number of different sources (e.g., yeast plasmids and viruses). Some vectors, referred to as shuttle vectors, are capable of replicating in both bacteria and eukaryotes. The nucleic acid from which the vector is derived is usually greatly reduced in size, such that only those genes essential for its autonomous replication remain. This reduction in size enables the vectors to accommodate large segments of foreign DNA. Examples of suitable vectors into which nucleic acid encoding PSF (e.g., PSF1, PSFa, or PSFb) or PSFL polypeptide can be inserted include, but are not limited to, pCGS, pBR322, pUC18, pUC19, pHSV-106, pJS97, pJS98, M13 mp18, M13 mp19, pSPORT 1, pgem, pSPORT 2, pSV, SPORT 1, pbluescript II, 8ZapII, 8gt10, 8gt11, 8gt22A, and 8ZIPLOX. Other suitable vectors will be obvious to one skilled in the art.

The vector of the present invention may be introduced into a host cell. Accordingly, the present invention further provides a host cell transformed with the vector of the present invention. The term “host cell”, as used herein, means the bacterial or eukaryotic cell into which the vector is introduced. The term “transform” denotes the introduction of a vector into a bacterial or eukaryotic host cell. Additionally, as used herein, the term “introduction” is a general term indicating that one of a variety of means has been used to allow the vector to enter the intracellular environment of the host cell in such a way that the nucleic acid exists in stable form therein, and may be expressed therein. As such, it encompasses transformation of bacterial cells, as well as transfection, transduction, and related methods in eukaryotic cells. The vector of the present invention may exist in integrated or unintegrated form within the host cell. When in unintegrated form, the vector is capable of autonomous replication.

Any one of a number of suitable bacterial or eukaryotic host cells may be transformed with the vector of the present invention. Examples of suitable host cells are known to one skilled in the art, and include, without limitation, bacterial cells, such as Escherichia coli (strains c600, c600hfl, HB101, LE392, Y1090, JM103, JM109, JM01, JM107, Y1088, Y1089, Y1090, Y1090(ZZ), DM1, PHIOB, DH115, DH1125, RR1, TB1, and SURE), Bacillus subtilis, Agrobacterium tumefaciens, and Bacillus megaterium; eukaryotic cells, such as Pichia pastoris, Chlamydomonas reinhardtii, Cryptococcus neoformans, Neurospora crassa, Podospora anserina, Saccharomyces cerevisiae, Saccharomyces pombe, and Uncinula necator; cultured insect cells; cultured chicken fibroblasts; cultured hamster cells; cultured human cells, such as HT1080, MCF7, and 143B; and cultured mouse cells, such as EL4 and NIH3T3 cells.

Some bacterial and eukaryotic vectors have been engineered so that they are capable of expressing inserted nucleic acids to high levels within the host cell. An “expression cassette” or “expression control sequence”, comprising nucleic acid encoding a PSF polypeptide (e.g., PSF1, PSFa, or PSFb) or a PSFL polypeptide operably linked to, or under the control of, transcriptional and translational regulatory elements (e.g., a promoter, ribosome binding site, operator, or enhancer), can be made and used for expression of PSF protein (e.g., the PSF1, PSFa, or PSFb isoform) or PSFL protein in vitro or in vivo. As used herein, “expression” refers to the ability of the vector to transcribe the inserted nucleic acid into mRNA, so that synthesis of the protein encoded by the inserted nucleic acid can occur. The choice of regulatory elements employed may vary, depending on such factors as the host cell to be transformed and the desired level of expression.

For example, in vectors used for the expression of a gene in a bacterial host cell such as Escherichia coli, the lac operator-promoter or the tac promoter is often used. Eukaryotic vectors use promoter-enhancer sequences of viral genes, especially those of tumor viruses. Several promoters for use in mammalian cells are known in the art. Examples of these promoters include, without limitation, the phosphoglycerate (PGK) promoter, the simian virus 40 (SV40) early promoter, the Rous sarcoma virus (RSV) promoter, the adenovirus major late promoter (MLP), and the human cytomegalovirus (CMV) immediate early 1 promoter. However, any promoter that facilitates suitable expression levels can be used in the present invention. Inducible promoters (e.g., those obtained from the heat shock gene, metallothionine gene, beta-interferon gene, or steroid hormone responsive genes, including, without limitation, the lac operator-promoter in E. coli and metallothionine or mouse mammary tumor virus promoters in eukaryotic cells) may be useful for regulating transcription based on external stimuli.

Vectors suitable for expressing in a host cell nucleic acid encoding PSF (e.g., PSF1, PSFa, or PSFb) or PSFL protein are well-known to one skilled in the art, and include pET-3d (Novagen, Inc., Madison, Wis.), pProEx-1 (Life Technologies, Inc., Gaithersburg, Md.), pFastBac 1 (Life Technologies), pSFV (Life Technologies), pcDNA II (Invitrogen Corporation, Carlsbad, Calif.), pSL301 (Invitrogen), pSE280 (Invitrogen), pSE380 (Invitrogen), pSE420 (Invitrogen), pTrcHis A,B,C (Invitrogen), pRSET A,B,C (Invitrogen), pYES2 (Invitrogen), pAC360 (Invitrogen), pVL1392 and pV11392 (Invitrogen), pCDM8 (Invitrogen), pcDNA I (Invitrogen), pcDNA I(amp) (Invitrogen), pZeoSV (Invitrogen), pcDNA 3 (Invitrogen), pRc/CMV (Invitrogen), pRc/RSV (Invitrogen), pREP4 (Invitrogen), pREP7 (Invitrogen), pREP8 (Invitrogen), pREP9 (Invitrogen), pREP10 (Invitrogen), pCEP4 (Invitrogen), pEBVHis (Invitrogen), and 8Pop6. Other vectors will be apparent to one skilled in the art.

The vector of the present invention may be introduced into a host cell using conventional procedures known in the art, including, without limitation, electroporation, DEAE Dextran transfection, calcium phosphate transfection, monocationic liposome fusion, polycationic liposome fusion, protoplast fusion, creation of an in vivo electrical field, DNA-coated microprojectile bombardment, injection with recombinant replication-defective viruses, homologous recombination, in vivo gene therapy, ex vivo gene therapy, viral vectors, and naked DNA transfer, and any combination thereof. For the purposes of gene transfer into a host cell, tissue, or subject, a recombinant vector containing nucleic acid encoding PSF (e.g., PSF1, PSFa, or PSFb) may be combined with a sterile aqueous solution that is preferably isotonic with the blood of the recipient. Such formulations may be prepared by suspending the recombinant vector in water containing physiologically-compatible substances, such as sodium chloride, glycine, and the like, and having buffered pH compatible with physiological conditions, to produce an aqueous solution, then rendering the solution sterile. In a preferred embodiment of the invention, the recombinant vector is combined with a 20-25% sucrose-in-saline solution, in preparation for introduction into a mammal.

The present invention further provides a method for making recombinant PSF polypeptide (including the PSF1, PSFa, and PSFb isoforms) or PSFL polypeptide, comprising the steps of: (a) introducing into a suitable bacterial or eukaryotic host cell a nucleic acid sequence encoding PSF (e.g., PSF1, PSFa, or PSFb) or PSFL protein, or a nucleic acid that hybridizes under high- or moderate-stringency conditions to a second nucleic acid that is complementary to the nucleotide sequence of SEQ ID NO:1, SEQ ID NO:2, SEQ ID NO:3, SEQ ID NO:7, SEQ ID NO:9, SEQ ID NO:11, SEQ ID NO:13, SEQ ID NO:15, SEQ ID NO:17, SEQ ID NO:18, or SEQ ID NO:20, or to a continuous fragment thereof; (b) maintaining the host cell under conditions such that the nucleic acid sequence is expressed to produce the PSF (e.g., the PSF1, PSFa, or PSFb isoform) or PSFL polypeptide; and (c) recovering the recombinant PSF or PSFL polypeptide from the culture medium, from the host cells, or from cell lysate. As used herein, the term “recombinant” refers to PSF polypeptide (e.g., the PSF1, PSFa, or PSFb isoform) or PSFL polypeptide produced by purification from a host cell transformed with a vector capable of directing its expression to a high level. In the method of the present invention, a nucleic acid sequence encoding PSF polypeptide (e.g., PSF1, PSFa, or PSFb) or PSF polypeptide may be introduced into a suitable host cell by any of the above-described methods. In a preferred embodiment of the invention, the polypeptide is human PSF.

A variety of methods of growing host cells transformed with a vector are known to those skilled in the art. The type of host cell (i.e., bacterial or eukaryotic) is the primary determinant of both the method to be utilized and the optimization of specific parameters relating to such factors as temperature, trace nutrients, humidity, and growth time. Depending on the vector used, the host cells may have to be induced by the addition of a specific compound at a certain point in the growth cycle, in order to initiate expression of the nucleic acid contained in the vector. Examples of compounds used to induce expression of the nucleic acid contained in the vector are known to one skilled in the art, and include, without limitation, IPTG, zinc, and dexamethasone. Using standard methods of protein isolation and purification, such as ammonium sulfate precipitation and subsequent dialysis to remove salt, followed by fractionation according to size, charge of the protein at specific pH values, affinity methods, etc., recombinant PSF polypeptide (e.g., the PSF1, PSFa, or PSFb isoform) or PSFL polypeptide may be extracted from suitable host cells transformed with a vector capable of expressing nucleic acid encoding PSF polypeptide (e.g., PSF1, PSFa, or PSFb) or PSFL polypeptide, respectively.

It is also within the confines of the present invention to provide a transgenic non-human animal whose genome comprises a disruption in the PSF gene (e.g., PSF1, PSFa, or PSFb) or PSFL gene, or a transgenic non-human animal that overexpresses PSF (e.g., the PSF1, PSFa, or PSFb isoform) or PSFL protein. The PSF and PSFL genes are known to exist in non-human animals, particularly mice. Although the non-human animal may be any suitable animal (e.g., cat, cattle, dog, horse, goat, rodent, and sheep), it is preferably a rodent. More preferably, the non-human animal is a rat or a mouse. The transgenic non-human animal of the present invention may be produced by a variety of techniques for genetically engineering transgenic animals, including those known in the art. In one embodiment of the present invention, the transgenic animal contains a host cell transformed with a vector, wherein the vector comprises a nucleic acid sequence encoding a PSF or PSFL polypeptide.

As used herein, the term “transgenic non-human animal” refers to a genetically-engineered non-human animal, produced by experimental manipulation, whose genome has been altered by introduction of a transgene. As further used herein, the term “transgene” refers to a nucleic acid (e.g., DNA, a gene, or a fragment thereof) that has been introduced into the genome of an animal by experimental manipulation, wherein the introduced gene is not endogenous to the animal, or is a modified or mutated form of a gene that is endogenous to the animal. The modified or mutated form of an endogenous gene may be produced through human intervention (e.g., by introduction of a point mutation, introduction of a frameshift mutation, deletion of a portion or fragment of the endogenous gene, insertion of a selectable marker gene, insertion of a termination codon, etc.). A transgenic non-human animal may be produced by several methods involving human intervention, including, without limitation, introduction of a transgene into an embryonic stem cell, newly-fertilized egg, or early embryo of a non-human animal; integration of a transgene into a chromosome of the somatic and/or germ cells of a non-human animal; and any methods described herein.

In one embodiment, the transgenic animal of the present invention has a genome in which the PSF or PSFL gene has been selectively inactivated, resulting in a disruption in its endogenous PSF or PSFL gene. As used herein, a “disruption” refers to a mutation (i.e., a permanent, transmissable change in genetic material) in the PSF or PSFL gene that prevents normal expression of functional PSF or PSFL protein (e.g., it results in expression of a mutant PSF or PSFL protein; it prevents expression of a normal amount of PSF or PSFL protein; or it prevents expression of PSF or PSFL protein). Examples of a disruption include, without limitation, a point mutation, introduction of a frameshift mutation, deletion of a portion or fragment of the endogenous gene, insertion of a selectable marker gene, and insertion of a termination codon. As used herein, the term “mutant” refers to a gene (or its gene product) which exhibits at least one modification in its sequence (or its functional properties) as compared with the wild-type gene (or its gene product). In contrast, the term “wild-type” refers to the characteristic genotype (or phenotype) for a particular gene (or its gene product), as found most frequently in its natural source (e.g., in a natural population). A wild-type animal, for example, expresses functional PSF or PSFL protein.

Selective inactivation in the transgenic non-human animal of the present invention may be achieved by a variety of methods, and may result in either a heterozygous disruption (wherein one PSF allele or one PSFL allele is disrupted, such that the resulting transgenic animal is heterozygous for the mutation) or a homozygous disruption (wherein both PSF or PSFL alleles are disrupted, such that the resulting transgenic animal is homozygous for the mutation). In one embodiment of the present invention, the endogenous PSF or PSFL gene of the transgenic animal is disrupted through homologous recombination with a nucleic acid sequence that encodes a region common to PSF or PSFL gene products. By way of example, the disruption through homologous recombination may generate a knockout mutation in the PSF or PSFL gene, particularly a knockout mutation wherein at least one deletion has been introduced into at least one exon of the PSF or PSFL gene. Additionally, a disruption in the PSF or PSFL gene may result from insertion of a heterologous selectable marker gene into the endogenous PSF or PSFL gene.

The method for creating a transgenic non-human animal having a knockout mutation in its PSF or PSFL gene may comprise the following steps: (a) generating a PSF or PSFL targeting vector; (b) introducing the PSF or PSFL targeting vector into a recipient cell of a non-human animal, to produce a treated recipient cell; (c) introducing the treated recipient cell into a blastocyst of a non-human animal, to produce a treated blastocyst; (d) introducing the treated blastocyst into a pseudopregnant non-human animal; (e) allowing the transplanted blastocyst to develop to term; (f) identifying a transgenic non-human animal whose genome comprises a knockout disruption in its endogenous PSF or PSFL gene; and (g) breeding the transgenic non-human animal to obtain a transgenic non-human animal exhibiting decreased expression of PSF or PSFL protein relative to wild-type. It is also within the confines of the present invention to provide a transgenic non-human animal that overexpresses PSF polypeptide (e.g., the PSF1, PSFa, or PSFb isoform) or PSFL polypeptide.

After the transgenic animal of the present invention (e.g., a transgenic non-human animal whose genome comprises a disruption in the PSF or PSFL gene or a transgenic non-human animal that overexpresses PSF or PSFL) has been produced, it may be analyzed to determine if the transgene has resulted in a pathology (e.g., excess production of amyloid-beta and/or the accumulation of neuritic plaques or neurofibrillary tangles). If pathologies do not develop in the animal, the transgenic animal may be crossed with another transgenic animal that does develop pathologies, to determine whether the presence of the transgene accelerates the pathology in question. For example, the inventors believe that PSF and PSFL protein may be associated with such pathologies as neuritic plaques and neurofibrillary tangles.

As disclosed herein, the inventors have determined that certain molecules that stabilize presenilin and/or nicastrin, referred to herein as “presenilin-stabilizing molecules”, modulate amyloid-beta production in cells. Accordingly, the present invention further provides a method for decreasing amyloid-beta production in a cell, comprising decreasing activity of a presenilin-stabilizing molecule in the cell. The cell may include any mammalian cell, but is preferably a cell of the central nervous system (CNS). Examples of CNS cells include, without limitation, astrocytes, ganglion cells, glial cells, granule cells, neuroglial cells, neuronal cells or neurons, oligodendrocytes, Schwann cells, and stellate cells. Examples of presenilin-stabilizing molecules include, without limitation, presenilin stabilization factor (PSF) and presenilin stabilization factor-like protein (PSFL), as disclosed above.

Unless otherwise indicated, “PSF” includes both a PSF protein, as characterized herein, and a “PSF analogue”. A “PSF analogue” is a functional variant of the PSF protein, having PSF-protein biological activity, that has 60% or greater (preferably, 70% or greater) amino-acid-sequence homology with the PSF protein, as well as a fragment of the PSF protein having PSF-protein biological activity. As further used herein, the term “PSF-protein biological activity” refers to protein activity which modulates amyloid-beta production, and stabilization of presenilin and nicastrin, as disclosed herein. PSF may be produced synthetically or recombinantly, or may be isolated from native cells; however, it is preferably produced recombinantly, using conventional techniques and cDNA encoding PSF, as disclosed herein.

Additionally, unless otherwise indicated, “PSFL” includes both a PSFL protein, as characterized herein, and a “PSFL analogue”. A “PSFL analogue” is a functional variant of the PSFL protein, having PSFL-protein biological activity, that has 60% or greater (preferably, 70% or greater) amino-acid-sequence homology with the PSFL protein, as well as a fragment of the PSFL protein having PSFL-protein biological activity. As further used herein, the term “PSFL-protein biological activity” refers to protein activity which modulates amyloid-beta production, and stabilization of presenilin and nicastrin, as disclosed herein. PSFL may be produced synthetically or recombinantly, or may be isolated from native cells; however, it is preferably produced recombinantly, using conventional techniques and cDNA encoding PSFL, as disclosed herein.

The method of the present invention, wherein amyloid-beta production is decreased in a cell by decreasing activity of a presenilin-stabilizing molecule in the cell, may be practiced in vitro, or in vivo in a subject. As used herein, the term “decreasing activity of a presenilin-stabilizing molecule” means attenuating, decreasing, or inhibiting one or more functions of the presenilin-stabilizing molecule, including, without limitation, the following: stabilizing presenilin or nicastrin in a cell, stabilizing a gamma-secretase complex in a cell, and enhancing or inducing gamma-secretase activity in the cell. Thus, a decrease in activity of a presenilin-stabilizing molecule in a cell effects a decrease in amyloid-beta production in the cell by one or more of the following biological processes: destabilization of presenilin or nicastrin in the cell, destabilization of a gamma-secretase complex in the cell, and inhibition of gamma-secretase activity in the cell. A decrease in amyloid-beta production and a decrease in activity of a presenilin-stabilizing molecule in a cell may be detected by known procedures, including any of the methods, molecular procedures, and assays disclosed herein.

In accordance with the methods of the present invention, activity of a presenilin-stabilizing molecule in a cell may be decreased by targeting the presenilin-stabilizing molecule directly (e.g., by disabling, disrupting, or inactivating the one or more functions of the presenilin-stabilizing molecule in the cell, or by diminishing the amount of the presenilin-stabilizing molecule in the cell). Additionally, activity of a presenilin-stabilizing molecule in a cell may be decreased indirectly, by targeting an enzyme or other endogenous molecule that regulates or modulates the functions or levels of presenilin-stabilizing molecules in the cell. Preferably, activity of a presenilin-stabilizing molecule in the cell is decreased by at least 10% in the method of the present invention. More preferably, activity of the presenilin-stabilizing molecule is decreased by at least 20%.

Activity of a presenilin-stabilizing molecule in a cell may be decreased by directly or indirectly inhibiting one or more functions of the presenilin-stabilizing molecule in the cell (e.g., by the modulation or regulation of proteins that interact with the presenilin-stabilizing molecule). In the method of the present invention, a presenilin-stabilizing molecule in a cell may be inhibited, for example, by contacting the cell with a small molecule or protein mimetic that inhibits the presenilin-stabilizing molecule and/or that is reactive with the presenilin-stabilizing molecule. Activity of a presenilin-stabilizing molecule in a cell also may be decreased by directly or indirectly causing, inducing, or stimulating the downregulation of expression of the presenilin-stabilizing molecule within the cell, thereby decreasing the levels of the presenilin-stabilizing molecule in the cell.

Accordingly, in one embodiment of the present invention, activity of the presenilin-stabilizing molecule is decreased in the cell by contacting the cell with an inhibitor of a presenilin-stabilizing molecule. As used herein, an “inhibitor” shall include, without limitation, a protein, polypeptide, peptide, nucleic acid (including genomic DNA, cDNA, antisense DNA, mRNA, dsRNA, siRNA, ssRNA, or antisense RNA), antibody (monoclonal and polyclonal, as described above), Fab fragment (as described above), F(ab′)₂ fragment (as described above), molecule, compound, antibiotic, drug, and any combinations thereof. Furthermore, inhibitors of the presenilin-stabilizing molecules may be agents reactive with PSF or PSFL, as defined above. In one embodiment of the invention, the inhibitor is dsRNA specific for the presenilin-stabilizing molecule. The inhibitor may be contacted with the cell in an amount effective to decrease amyloid-beta production in the cell. This amount may be readily determined by the skilled artisan, based upon known procedures and methods disclosed herein.

Activity of a presenilin-stabilizing molecule in a cell also may be decreased by directly or indirectly causing, inducing, or stimulating the downregulation of expression of the presenilin-stabilizing molecule within the cell. Accordingly, in another embodiment of the present invention, activity of the presenilin-stabilizing molecule is decreased in a cell by contacting the cell with a modulator of expression of the presenilin-stabilizing molecule, in an amount effective to decrease amyloid-beta production in the cell. As used herein, the “modulator” shall include, without limitation, a protein, polypeptide, peptide, nucleic acid (including DNA or RNA), antibody, Fab fragment, F(ab′)₂ fragment, molecule, compound, antibiotic, drug, or an agent reactive with PSF or PSFL, as defined herein, that down-regulates expression of the presenilin-stabilizing molecule.

Inhibitors and modulators of presenilin-stabilizing molecules may be identified using simple screening assays well known in the art or disclosed herein. For example, to screen for candidate modulators of a presenilin-stabilizing molecule (e.g., PSF or PSFL), mammalian cells (e.g., human HEK293 cells) may be plated onto microtiter plates, and then contacted with a library of drugs. Any resulting decrease in, or downregulation of, expression of the presenilin-stabilizing molecule then may be detected using nucleic acid hybridization and/or immunological techniques known in the art, including an ELISA. Additionally, it is within the confines of the present invention that the modulator of expression disclosed herein may be linked to another agent, or administered in combination with another agent, such as a drug or a ribozyme, in order to increase the efficacy of targeting and/or facilitate the decrease in amyloid-beta production.

In the method of the present invention, an inhibitor of a presenilin-stabilizing molecule may be contacted with a cell by introducing the inhibitor into the cell. Where contacting is effected in vitro, the inhibitor may be added directly to the culture medium. Alternatively, the inhibitor may be contacted with a cell in vivo in a subject by introducing the inhibitor into, or administering the inhibitor to, the subject, preferably by introducing the inhibitor into cells of the subject. The subject may be any animal, but is preferably a mammal (e.g., humans, domestic animals, and commercial animals). More preferably, the subject is a human. The amount of inhibitor to be used is an amount effective to decrease amyloid-beta production in the cell, as defined above, and may be readily determined by the skilled artisan.

The inhibitor of the present invention may be contacted with a cell, either in vitro or in vivo in a subject, by known techniques used for the introduction and administration of nucleic acids, proteins, and other drugs, including, for example, injection and transfusion. When target cells are localized to a particular portion of the body of the subject, it may be desirable to introduce the inhibitor directly to the cells by injection or by some other means (e.g., by introducing the inhibitor into the blood or another body fluid). The inhibitor may be introduced into cells, in vitro or in vivo, using conventional procedures known in the art, including, without limitation, electroporation, DEAE Dextran transfection, calcium phosphate transfection, monocationic liposome fusion, polycationic liposome fusion, protoplast fusion, creation of an in vivo electrical field, DNA-coated microprojectile bombardment, injection with recombinant replication-defective viruses, homologous recombination, in vivo gene therapy, ex vivo gene therapy, viral vectors, and naked DNA transfer, or any combination thereof. Recombinant viral vectors suitable for gene therapy include, but are not limited to, vectors derived from the genomes of viruses such as retrovirus, HSV, adenovirus, adeno-associated virus, Semiliki Forest virus, cytomegalovirus, and vaccinia virus.

Where the inhibitor of a presenilin-stabilizing molecule is a protein, it may be introduced into a cell directly, in accordance with conventional techniques and methods disclosed herein. Additionally, a protein inhibitor may be introduced into a cell indirectly, by introducing into the cell a nucleic acid encoding the inhibitor, in a manner permitting expression of the protein inhibitor. The amount of nucleic acid to be used is an amount sufficient to express an amount of protein inhibitor effective to decrease amyloid-beta production. These amounts may be readily determined by the skilled artisan.

It is also within the confines of the present invention that a nucleic acid encoding a protein inhibitor of a presenilin-stabilizing molecule may be introduced into suitable cells in vitro, using conventional procedures, to achieve expression of inhibitor protein in the cells. Cells expressing inhibitor protein then may be introduced into a subject to inhibit proliferation of astrocytes in vivo. In such ex vivo gene therapy approaches, the cells are preferably removed from the subject, subjected to DNA techniques to incorporate nucleic acid encoding the inhibitor, and then reintroduced into the subject.

In accordance with the method of the present invention, an inhibitor of a presenilin-stabilizing molecule may be administered to a human or animal subject by known procedures, including, without limitation, oral administration, parenteral administration, transdermal administration, and administration through an osmotic mini-pump. Preferably, the inhibitor is administered parenterally, by intracranial, intraspinal, intrathecal, or subcutaneous injection. The inhibitor of the present invention also may be administered to a subject in accordance with any of the above-described methods for effecting in vivo contact between cells and inhibitors of presenilin-stabilizing molecules.

For oral administration, the inhibitor formulation may be presented as capsules, tablets, powders, granules, or as a suspension. The formulation may have conventional additives, such as lactose, mannitol, corn starch, or potato starch. The formulation also may be presented with binders, such as crystalline cellulose, cellulose derivatives, acacia, corn starch, or gelatins. Additionally, the formulation may be presented with disintegrators, such as corn starch, potato starch, or sodium carboxymethylcellulose. The formulation also may be presented with dibasic calcium phosphate anhydrous or sodium starch glycolate. Finally, the formulation may be presented with lubricants, such as talc or magnesium stearate.

For parenteral administration (i.e., administration by injection through a route other than the alimentary canal), the inhibitor may be combined with a sterile aqueous solution that is preferably isotonic with the blood of the subject. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulations may be presented in unit or multi-dose containers, such as sealed ampoules or vials. The formulation may be delivered by any mode of injection, including, without limitation, epifascial, intracapsular, intracranial, intracutaneous, intrathecal, intramuscular, intraorbital, intraperitoneal, intraspinal, intrasternal, intravascular, intravenous, parenchymatous, subcutaneous, or sublingual.

For transdermal administration, the inhibitor may be combined with skin penetration enhancers, such as propylene glycol, polyethylene glycol, isopropanol, ethanol, oleic acid, N-methylpyrrolidone, and the like, which increase the permeability of the skin to the inhibitor, and permit the inhibitor to penetrate through the skin and into the bloodstream. The inhibitor/enhancer compositions also may be further combined with a polymeric substance, such as ethylcellulose, hydroxypropyl cellulose, ethylene/vinylacetate, polyvinyl pyrrolidone, and the like, to provide the composition in gel form, which may be dissolved in solvent, such as methylene chloride, evaporated to the desired viscosity, and then applied to backing material to provide a patch. The inhibitor may be administered transdermally at the site in the subject where the neurodegeneration is localized. Alternatively, the inhibitor may be administered transdermally at a site other than the affected area, in order to achieve systemic administration.

The inhibitor of the present invention also may be released or delivered from an osmotic mini-pump or other time-release device. The release rate from an elementary osmotic mini-pump may be modulated with a microporous, fast-response gel disposed in the release orifice. An osmotic mini-pump would be useful for controlling release, or targeting delivery, of the inhibitor.

The ability of presenilin-stabilizing molecules to modulate amyloid-beta production in a cell renders their inhibitors particularly useful for treating conditions associated with an excess of amyloid-beta production, particularly neurodegeneration. As used herein, the term “neurodegeneration” means a condition of deterioration of a neuron, wherein the neuron changes to a lower or less functionally-active form. It is believed that, by decreasing amyloid-beta production in neurons, PSF, PSFL, and other presenilin-stabilizing molecules will be useful for the treatment of neurodegeneration. It is further believed that these molecules would be effective either alone or in combination with therapeutic agents, such as chemotherapeutic agents or antiviral agents, which are typically used in the treatment of neurodegeneration.

Accordingly, the present invention provides a method for treating neurodegeneration in a subject in need of treatment, by contacting cells (preferably, cells of the CNS) in the subject with an amount of an inhibitor of a presenilin-stabilizing molecule effective to decrease amyloid-beta production in the cells, thereby treating the neurodegeneration. Examples of neurodegeneration which may be treated by the method of the present invention include, without limitation, Alzheimer's disease, amyotrophic lateral sclerosis (Lou Gehrig's Disease), Binswanger's disease, corticobasal degeneration (CBD), dementia lacking distinctive histopathology (DLDH), frontotemporal dementia (FTD), Huntington's chorea, multiple sclerosis, myasthenia gravis, Parkinson's disease, Pick's disease, and progressive supranuclear palsy (PSP). In a preferred embodiment of the present invention, the neurodegeneration is Alzheimer's disease. In a further embodiment of the present invention, the Alzheimer's disease is early-onset familial Alzheimer's disease.

The present invention also provides a method for treating neurodegeneration in a subject in need of treatment, comprising administering to the subject an inhibitor of presenilin stabilization factor (PSF) or an inhibitor of presenilin stabilization factor-like protein (PSFL) in an amount effective to treat the neurodegeneration. As used herein, the phrase “effective to treat the neurodegeneration” means effective to ameliorate or minimize the clinical impairment or symptoms of the neurodegeneration. For example, where the neurodegeneration is Alzheimer's disease, the clinical impairment or symptoms of the neurodegeneration may be ameliorated or minimized by reducing the production of amyloid-beta and the development of senile plaques and neurofibrillary tangles, thereby minimizing or attenuating the progressive loss of cognitive function. The amount of inhibitor effective to treat neurodegeneration in a subject in need of treatment will vary depending upon the particular factors of each case, including the type of neurodegeneration, the stage of the neurodegeneration, the subject's weight, the severity of the subject's condition, and the method of administration. This amount can be readily determined by the skilled artisan.

The present invention further provides pharmaceutical compositions for use in decreasing amyloid-beta production, comprising a pharmaceutically-acceptable carrier and an inhibitor (either protein or nucleic acid) of a presenilin-stabilizing molecule (e.g., PSF or PSFL). Examples of acceptable pharmaceutical carriers, formulations of the pharmaceutical compositions, and methods of preparing the formulations are described above. The pharmaceutical compositions may be useful for administering the inhibitor (protein or nucleic acid) of the present invention to a subject to treat a variety of disorders, including neurodegeneration, as disclosed herein. The inhibitor is provided in an amount that is effective to treat the disorder (e.g., neurodegeneration) in a subject to whom the pharmaceutical composition is administered. This amount may be readily determined by the skilled artisan, as described above.

As described herein, the inventors have determined that the suppression of Drosophila or human forms of PSF and PSFL abrogates the γ-secretase-mediated generation of Aβ, and also disrupts the stability of both presenilin and nicastrin. Furthermore, using affinity isolation experiments, the inventors have demonstrated that PSF forms a complex with nicastrin and presenilin 1. Thus, as shown herein, PSF is required for γ-secretase activity and stabilization, and for the stabilization of presenilin and nicastrin. According, the present invention further provides a method for destabilizing presenilin or nicastrin in a cell, comprising decreasing activity of a presenilin-stabilizing molecule (e.g., PSF or PSFL) in the cell. The present invention also provides a method for destabilizing a gamma-secretase complex in a cell, comprising decreasing activity of a presenilin-stabilizing molecule (e.g., PSF or PSFL) in the cell. Moreover, the present invention provides a method for inhibiting activity of gamma-secretase in a cell, comprising decreasing activity of a presenilin-stabilizing molecule (e.g., PSF or PSFL) in the cell. Methods for decreasing activity of a presenilin-stabilizing molecule in a cell have been described above.

As disclosed herein, the inventors have also determined that certain peptides of the rhomboid protein family modulate amyloid-beta production in cells. Accordingly, the present invention further provides a method for decreasing amyloid-beta production in a cell, comprising increasing activity of a rhomboid peptide in the cell. The cell may include any mammalian cell, but is preferably a cell of the central nervous system (CNS). Examples of rhomboid peptides include, without limitation, rhomboid 1, rhomboid 2, rhomboid 3, rhomboid 4, rhomboid 5, rhomboid 6, and rhomboid 7. Preferably, the rhomboid peptides of the present invention are rhomboid 1 and rhomboid 7.

Unless otherwise indicated, “rhomboid 1” includes a rhomboid 1 protein (SEQ ID NO:72), a “rhomboid 1 analogue”, and human homologues thereof. Rhomboid 1 is known to be conserved throughout evolution, from archaea to humans (Urban et al., Drosophila rhomboid-1 defines a family of putative intramembrane serine proteases. Cell, 107(2):173-82, 2001). A “rhomboid 1 analogue” is a functional variant of the rhomboid 1 protein, having rhomboid-1-protein biological activity, that has 60% or greater (preferably, 70% or greater) amino-acid-sequence homology with the rhomboid 1 protein, as well as a fragment of the rhomboid 1 protein having rhomboid-1-protein biological activity. As further used herein, the term “rhomboid-1-protein biological activity” refers to protein activity which modulates amyloid-beta production, and stabilization of presenilin and nicastrin, as disclosed herein. Rhomboid 1 may be produced synthetically or recombinantly, or may be isolated from native cells; however, it is preferably produced recombinantly, using conventional techniques and cDNA encoding rhomboid 1, as depicted herein (SEQ ID NO:71).

Furthermore, unless otherwise indicated, “rhomboid 7” includes both a rhomboid 7 protein (SEQ ID NO:74), a “rhomboid 7 analogue”, and human homologues thereof. A “rhomboid 7 analogue” is a functional variant of the rhomboid 7 protein, having rhomboid-7-protein biological activity, that has 60% or greater (preferably, 70% or greater) amino-acid-sequence homology with the rhomboid 7 protein, as well as a fragment of the rhomboid 7 protein having rhomboid-7-protein biological activity. As further used herein, the term “rhomboid-7-protein biological activity” refers to protein activity which modulates amyloid-beta production, and stabilization of presenilin and nicastrin, as disclosed herein. Rhomboid 7 may be produced synthetically or recombinantly, or may be isolated from native cells; however, it is preferably produced recombinantly, using conventional techniques and cDNA encoding rhomboid 7, as depicted herein (SEQ ID NO:73).

The method of decreasing amyloid-beta production in a cell, by increasing activity of a rhomboid peptide in the cell, may be practiced either in vitro, or in vivo in a subject. As used herein, the term “increasing activity of a rhomboid peptide” means enhancing or increasing one or more functions of the rhomboid peptide, including, without limitation, the following: destabilizing presenilin or nicastrin in a cell, destabilizing a gamma-secretase complex in a cell, and attenuating, decreasing, or inhibiting gamma-secretase activity in the cell. Thus, an increase in activity of a rhomboid peptide in a cell effects a decrease in amyloid-beta production in the cell by one or more of the following biological processes: destabilization of presenilin or nicastrin in the cell, destabilization of a gamma-secretase complex in the cell, and inhibition of gamma-secretase activity in the cell. A decrease in amyloid-beta production in a cell, and an increase in activity of a rhomboid peptide in a cell, may be detected by known procedures, including any of the methods, molecular procedures, and assays disclosed herein.

In accordance with the methods of the present invention, activity of a rhomboid peptide in a cell may be increased by targeting the rhomboid peptide directly (e.g., by activating, facilitating, or stimulating one or more functions of the rhomboid peptide in the cell, or by increasing the amount of the rhomboid peptide in the cell). Additionally, activity of a rhomboid peptide in a cell may be increased indirectly, by targeting an enzyme or other endogenous molecule that regulates or modulates the functions or levels of the rhomboid peptide in the cell. Preferably, activity of a rhomboid peptide in a cell is increased by at least 10% in the method of the present invention. More preferably, activity of the rhomboid peptide is increased by at least 20%.

Activity of a rhomboid peptide in a cell may be increased by directly or indirectly increasing levels of the rhomboid peptide in the cell. By way of example, the level of the rhomboid peptide in a cell may be increased by introducing the rhomboid peptide directly to the cell. Similarly, the level of the rhomboid peptide in a cell may be increased by introducing to the cell a nucleic acid sequence encoding the rhomboid peptide, in a manner permitting expression of the rhomboid peptide in the cell. Additionally, the level of rhomboid peptide in a cell, and, therefore, the activity of a rhomboid peptide in a cell, may be increased by directly or indirectly causing, inducing, or stimulating the upregulation of expression of the rhomboid peptide within the cell.

Accordingly, in one embodiment of the present invention, activity of a rhomboid peptide is increased in a cell by contacting the cell with the rhomboid peptide itself or with a modulator of the rhomboid peptide's expression. Examples of modulators of rhomboid peptide expression include, without limitation, a protein, polypeptide, peptide, nucleic acid (including genomic DNA, cDNA, antisense DNA, mRNA, dsRNA, siRNA, ssRNA, or antisense RNA), antibody (monoclonal and polyclonal, Fab fragment, F(ab′)₂ fragment, molecule, compound, antibiotic, drug, and any combinations thereof. Furthermore, modulators of rhomboid peptides may be agents reactive with rhomboid 1 or rhomboid 7. Modulators of the rhomboid peptide for use in the present invention include those drugs which induce upregulate expression of the rhomboid peptide in the cell, thereby increasing the levels of the rhomboid peptide in the cell. Such modulators may be identified using a simple screening assay, as described above.

In the method of the present invention, the rhomboid peptide or modulator may be contacted with the cell in an amount effective to decrease amyloid-beta production in the cell. This amount may be readily determined by the skilled artisan, based upon known procedures and methods disclosed herein. It is also within the confines of the present invention that the modulator of expression disclosed herein may be linked to another agent, or administered in combination with another agent, such as a drug or a ribozyme, in order to increase the efficacy of targeting and/or facilitate the decrease in amyloid-beta production.

Activity of a rhomboid peptide in a cell also may be increased by directly or indirectly activating, facilitating, or stimulating one or more functions of the rhomboid peptide in the cell (e.g., by the modulation or regulation of proteins that interact with the rhomboid peptide). A rhomboid peptide in a cell may be activated, for example, by contacting the cell with a small molecule or protein mimetic that stimulates the rhomboid peptide and/or that is reactive with the rhomboid peptide.

Where contacting with the cell is effected in vitro, the rhomboid peptide or modulator may be added directly to the culture medium, permitting introduction of the rhomboid peptide or modulator into the cell. Alternatively, the rhomboid peptide or modulator may be contacted with a cell in vivo in a subject by introducing the peptide or modulator into, or administering the peptide or modulator to, the subject. The amount of rhomboid peptide or modulator to be used is an amount effective to decrease amyloid-beta production in the cell. This amount may be readily determined by the skilled artisan. The rhomboid peptides and modulators of the present invention —which may be proteins, nucleic acids, or other molecules —may be contacted with a cell, either in vitro or in vivo in a subject, by known techniques used for the introduction and administration of nucleic acids, proteins, and other drugs, including those described herein.

The ability of rhomboid peptides to modulate amyloid-beta production in a cell renders them particularly useful for treating conditions associated with an excess of amyloid-beta production, particularly neurodegeneration. It is believed that, by decreasing amyloid-beta production in neurons, rhomboid 1, rhomboid 7, and other rhomboid peptides will be useful for the treatment of neurodegeneration. It is further believed that these molecules would be effective either alone or in combination with therapeutic agents, such as chemotherapeutic agents or antiviral agents, which are typically used in the treatment of neurodegeneration. Accordingly, the present invention provides a method for treating neurodegeneration in a subject in need of treatment, by contacting cells (preferably, cells of the CNS) in the subject with a rhomboid peptide (e.g., rhomboid 1 or rhomboid 7), or a modulator of the rhomboid peptide's expression, in an amount effective to decrease amyloid-beta production in the cells, thereby treating the neurodegeneration. This amount may be readily determined by the skilled artisan.

The present invention also provides a method for treating neurodegeneration in a subject in need of treatment, comprising administering to the subject a rhomboid peptide (e.g., rhomboid 1 or rhomboid 7), or a modulator of the rhomboid peptide's expression, in an amount effective to treat the neurodegeneration. The amount of peptide or modulator effective to treat neurodegeneration in the subject will vary depending upon the particular factors of each case, as described above. This amount may be readily determined by the skilled artisan.

The present invention also provides pharmaceutical compositions for use in decreasing amyloid-beta production, comprising rhomboid 1 protein and a pharmaceutically-acceptable carrier, rhomboid 1 nucleic acid and a pharmaceutically-acceptable carrier, rhomboid 7 protein and a pharmaceutically-acceptable carrier, or rhomboid 7 nucleic acid and a pharmaceutically-acceptable carrier. Examples of acceptable pharmaceutical carriers, formulations of the pharmaceutical compositions, and methods of preparing the formulations are described above. The pharmaceutical compositions may be useful for administering the rhomboid 1 or rhomboid 7 protein or nucleic acid of the present invention to a subject to treat a variety of disorders, including neurodegeneration, as disclosed herein. The rhomboid 1 or rhomboid 7 protein or nucleic acid is provided in an amount that is effective to treat the disorder (e.g., neurodegeneration) in a subject to whom the pharmaceutical composition is administered. This amount may be readily determined by the skilled artisan, as described above.

As further disclosed herein, the inventors have developed a novel assay system based on RNA interference (RNAi), and a novel method for identifying agents that modulate production of amyloid-beta in cells. The inventors' assay allows one to test the contribution of virtually any gene (and gene product) to the production of amyloid-beta, using cells, derived from the fruit fly, that have been engineered to express human APP and/or the presenilins. RNAi is a phenomenon of sequence-specific, post-transcriptional gene silencing, mediated by dsRNA. The inventors' studies have shown that RNAi of presenilin and nicastrin (a reported presenilin-binding protein) efficient represses amyloid-beta generation. This novel RNAi-based assay system will allow for the screening of novel cellular factors that specifically antagonize the phenotype associated with FAD mutant presenilins (e.g., amyloid-beta overproduction). Such findings may lead to novel therapies aimed at regulating Alzheimer's-disease-associated cellular changes (e.g., aberrant regulation of Aβ42 generation and regulation of total AP levels), which may carry great potential as effective treatments for Alzheimer's disease.

Accordingly, the present invention provides an in vitro system for identifying an agent that selectively modulates production of amyloid-beta or an amyloid-beta precursor (e.g., amyloid-beta precursor protein, APP), comprising Drosophila-derived S2 cells that express human APP, a human APP precursor or derivative, or a human presenilin. Such an in vitro system may be made by generating Drosophila-derived S2 cells that express human APP, a human APP precursor or derivative, or a human presenilin. The present invention further provides an in vitro system made by this method.

By way of example, Drosophila S2 cells may be cultured in Schneider's Drosophila medium (Gibco, Grand Island, N.Y.) supplemented with 10% fetal bovine serum (FBS), 100 units/ml penicillin, and 100 μg/ml streptomycin (Invitrogen Corporation, Carlsbad, Calif.) at a suitable temperature (e.g., 25° C.). Transient and stable S2 cell lines then may be generated by transfecting approximately 2-3×10⁶ cells, in a 6-well plate, with 2 μg of plasmid, using Effectene™ transfection reagent (Qiagen, Valencia, Calif.). After an appropriate amount of time (e.g., 60 h), cells may be harvested, and the level of expression may be checked. Stable S2 cell lines may be generated with the cotransfection of pCoHygro (Invitrogen) and/or pCoBlast (Invitrogen), and cultured in a medium supplemented with 100 μg/ml of hygromycin B (Roche Molecular Biochemicals, Indianapolis, Ind.) and/or 50 μg/ml of blasticidin S (Invitrogen). S2 cells (approximately 3×10⁶) may be cultured in 6-well plates using Drosophila expression system (DES) serum-free medium (Invitrogen Corporation, Carlsbad, Calif.).

A series of human PSF stable cell lines may be generated by transfecting stable 293 cells expressing human APP-695 (or APP-C99) in a dish (e.g., a 100-mm dish), with 5 μg of hPSF cloned in pEF6N5-His TOPO vector, using Superfect transfection reagent (Qiagen). Individual blasticidin-S-resistant colonies may be isolated, and then screened for PSF expression by Western blotting using V5 antibody (Invitrogen). Stable cell lines may be maintained in DMEM supplemented with 10% fetal bovine serum and penicillin/streptomycin, in the presence of 250 μg/ml G418 (Calbiochem, San Diego, Calif.) and 1 μg/ml of blasticidin S. Stable cell lines expressing human APP, or a precursor thereof, may be generated using a similar method.

Once the above-described method has been used to generate an in vitro system, or such an in vitro system has been obtained, the system then may be used to screen for agents that selectively modulate generation of amyloid-beta or an amyloid-beta precursor. For example, the S2 cells of the in vitro system may be contacted with dsRNA specific for a candidate protein product, and then analyzed in order to assess the ability of the candidate protein product to modulate production of amyloid-beta or an amyloid-beta precursor in the cells. Similarly, the S2 cells of the in vitro system may be contacted with a candidate agent, and then analyzed in order to assess the ability of the candidate agent to modulate production of amyloid-beta or an amyloid-beta precursor in the cell.

Accordingly, the present invention further provides a method for identifying an agent (e.g., a protein product) that modulates production of amyloid-beta or an amyloid-beta precursor, comprising the steps of: (a) obtaining or generating Drosophila-derived S2 cells that express human APP, a human APP precursor or derivative, or a human presenilin; (b) contacting the cells with dsRNA for a candidate agent (e.g., a protein product); and (c) assessing the ability of the dsRNA to modulate production of amyloid-beta or an amyloid-beta precursor in the cells. In this method of the invention, ability of the dsRNA to modulate production of amyloid-beta or an amyloid-beta precursor will be indicative that the candidate agent (the protein product) modulates production of amyloid-beta or an amyloid-beta precursor.

In one embodiment of the present invention, for example, a candidate cDNA fragment of suitable length (e.g., approximately 700 bp in length) may be reamplified —first, by PCR using a forward primer containing a 5′ T7 RNA polymerase binding site (e.g., GAA TTAATACGACTCACTATAGGGAGA (SEQ ID NO:30)) and a reverse primer containing a 3′ SP6 RNA polymerase binding site (e.g., ATTTAGGTGACACTATAGAAGCG (SEQ ID NO:31)), and, subsequently, by the specific sequences of the targeted gene. Examples of primers suitable for use in this reaction, without T7 and SP6 sequences, may be found in Table 1 herein.

Sense and antisense RNAs may be produced using MEGASCRIPT T7 and an SP6 high-yield transcription RNA kit (Ambion, Austin, Tex.). The generated sense and antisense RNAs may be annealed by incubation at a suitable temperature and for a suitable time (e.g., at 65° C. for 30 min), then cooled (e.g., at 37° C.). The double-stranded RNA (dsRNA) products may be precipitated (e.g., by ethanol), resuspended in nuclease-free water, and analyzed by gel electrophoresis (e.g., on 1% agarose). Small interference RNA (siRNA) may be obtained by IBA GmbH, Inc. (Goettingen, Germany). The synthetic RNA oligonucleotides may be gel-purified after deprotection, and used to generate an siRNA duplex, as previously described (Elbashir et al., Duplexes of 21-nucleotide RNAs mediate RNA interference in cultured mammalian cells. Nature, 411:494-98, 2001).

RNA interference (RNAi) experiments may be performed on S2 cells by adding dsRNA to the medium mixed with 7 μl of Lipofectamine 2000 (Invitrogen), in accordance with the manufacturer's instruction. Cells may be left for an appropriate time and at a suitable temperature (e.g., for 30 min, at room temperature), and then incubated for an appropriate time and at a suitable temperature (e.g., for 3 days at 25° C.). To ensure the effect of dsRNA, each RNA may be purified with RNeasy Mini Kit (Qiagen, Valencia, Calif.). RT PCR may be performed by SUPERSCRIPT™ One-Step RT-PCR with PLATINUM® Taq (Invitrogen). Transfection of siRNAs for target endogenous genes may be carried out using Oligofectamine (Invitrogen) (Elbashir et al., Duplexes of 21-nucleotide RNAs mediate RNA interference in cultured mammalian cells. Nature, 411:494-98, 2001). After an appropriate amount of time (e.g., 60 h), cells may be harvested and subjected to Western blotting to assess the ability of the dsRNA to modulate production of amyloid-beta.

Human RAPID-SCAN™ gene expression and human brain RAPID-SCAN™ gene expression panels (OriGene Technologies, Inc., Rockville, Md.) may be used to examine the tissue distribution and the spliced product of the candidate agent. PCR may be used to amplify the cDNAs. By way of example, the PCR schedule may consist of HotStartTaq polymerase activation at a suitable temperature and for a suitable time (e.g., at 94° C. for 10 min), followed by denaturation at a suitable temperature and for a suitable time (e.g., at 94° C. for 1 min), a first primer extension at a suitable temperature and for a suitable time (e.g., at 62° C. for 1 min), and a subsequent primer extension at a suitable temperature and for a suitable time (e.g., at 72° C. for 2.5 min).

At a suitable time post-transfection (e.g., 72 h), S2 cells then may be lysed (e.g., using a buffer consisting of 100 mM Tris-HCl, pH 7.4; 150 mM NaCl; 1% Triton X-100; 0.25% Nonidet P-40; and 2 mM EDTA) supplemented with protease inhibitor cocktail tablet (Roche Molecular Biochemicals, Indianapolis, Ind.)). After centrifugation (e.g., at 14.000×g for 10 min), the supernatant may be collected. The precipitates then may be washed, and the protein complex may be eluted (e.g., with a suitable buffer). The eluted protein complex may be mixed with 2×NuPAGE™ LDS sample buffer (Invitrogen Corporation, Carlsbad, Calif.), and subjected to Western blotting. Western-blot experiments may be carried out using either NuPAGE™ 4-12% Bis-Tris Gel or 4-20% Tris-Glycine Gel (Invitrogen). Primary antibodies may be used at appropriate dilutions.

The inventors' in vitro system and associated method for identifying agents that modulate production of amyloid-beta in a cell also may be useful in screening assays other than RNAi assays. Accordingly, the present invention further provides a method for identifying an agent that modulates production of amyloid-beta or an amyloid-beta precursor, comprising the steps of: (a) obtaining or generating Drosophila-derived S2 cells that express human APP, a human APP precursor or derivative, or a human presenilin; (b) contacting the cells with a candidate agent (e.g., a small molecule that may be capable of modulating expression of amyloid-beta or an amyloid-beta precursor); and (c) assessing the ability of the candidate agent to modulate production of amyloid-beta or an amyloid beta precursor in the cells.

The present invention also provides agents identified by the above-described methods and in vitro systems. As described above, the agent may be a protein product, identified through RNAi assays, or a small molecule, identified directly. In one embodiment of the invention, the agent or protein product decreases production of amyloid-beta. Such an agent may be useful in the treatment of conditions associated with an excess of amyloid-beta. Accordingly, the present invention further provides a method for treating neurodegeneration in a subject in need of treatment, by administering such an agent to the subject, in an amount effective to treat the neurodegeneration in the subject.

The present invention is described in the following Examples, which are set forth to aid in the understanding of the invention, and should not be construed to limit in any way the scope of the invention as defined in the claims which follow thereafter.

### EXAMPLES

### Example 1

### Plasmids

The expression construct encoding SPCT99HA (containing a Bip signal peptide and a C-terminal HA tag) was generated by High Fidelity PCR Master (Roche Molecular Biochemicals, Indianapolis, Ind.) using human APP-695 as a template and the following primers: (1) 5′-SPCT: GACTGCGATATCATGAAGTTATGCATATTAC TGGCCGTCGTGGCCTTTGTTGGCCTCTCGCTCGGGGATGCAGAATTCCGACATGA CTCAGG (SEQ ID NO:22); and (2) 3′-CTHA: GCCGACTCTAGACTAGAGGCTTGCAT AATCTGGCACATCATATGGATAGTTCTGCATCTGCTCAAAGAACTTGTAGG (SEQ ID NO:23). Bold letters denote the incorporated HA-tag sequence.

The generated PCR products were digested with EcoR V and Xba I, and subcloned into pAc5.1V5HisA (Invitrogen Corporation, Carlsbad, Calif.); the sequence was then verified. Drosophila presenilin with HA tag under the tubulin promoter was a generous gift from G. Struhl (Dr. Struhl's location? Please provide.). The Drosophila nicastrin expression construct was generated using the following primers: (1) Nic NF(s): CCCGGGGGTACCTCTTCGATGGAAATGCGTCTGAATGCGGC (SEQ ID NO:24); and (2) Nic CF(a): AAATTTGAATTCACCAAATAATGCGGCATTGCTTGC (SEQ ID NO:25). The PCR products, digested with EcoR I and Kpn I, were subcloned into pAc5.1V5HisA. Human PSF1, PSFa, and PSFb expression constructs were generated with the following primers, using a brain cDNA library (OriGene Technologies, Inc., Rockville, Md.): (1) HPSF1(s) common: GCCATGGGGGCTGCGGTGTTTTTCGGCTGC (SEQ ID NO:26); (2) HPSF1(a): GTCCAGGTAGTCAGTCCTTACACAAGAGCTGC (SEQ ID NO:27); (3) HPSFa(a): GTCCTTACACAAGAGGCTGCGCTGAATACTTC (SEQ ID NO:28); and (4) HPSFb(a): GTCCTCGGGTGGGATGCGCAGGGCAGAATAC (SEQ ID NO:29). Each PCR product was cloned directly with pEF/V5-His TOPO TA expression vector (Invitrogen), and the sequence was confirmed. The correctly amplified clone was chosen for further experiment.

### Example 2

### Cell Culture

Drosophila S2 cells were cultured in Schneider's Drosophila medium (Gibco, Grand Island, N.Y.) supplemented with 10% fetal bovine serum (FBS), 100 units/ml penicillin, and 100 μg/ml streptomycin (Invitrogen Corporation, Carlsbad, Calif.) at 25° C. Transient and stable S2 cell lines were generated by transfecting approximately 2-3×10⁶ cells, in a 6-well plate, with 2 μg of plasmid using Effectene™ transfection reagent (Qiagen, Valencia, Calif.). After 60 h, cells were harvested, and the level of expression was checked. Stable S2 cell lines were generated with the cotransfection of pCoHygro (Invitrogen) and/or pCoBlast (Invitrogen), and cultured in a medium supplemented with 100 μg/ml of hygromycin B (Roche Molecular Biochemicals, Indianapolis, Ind.) and/or 50 μg/ml of blasticidin S (Invitrogen).

A series of human PSF stable cell lines were generated by transfecting stable 293 cells expressing human APP-695, in a 100-mm dish, with 5 μg of hPSF cloned in pEF6/V5-His TOPO vector using Superfect transfection reagent (Qiagen). Individual blasticidin-S-resistant colonies were isolated, and then screened for PSF expression by Western blotting using V5 antibody (Invitrogen). Stable cell lines were maintained in DMEM supplemented with 10% fetal bovine serum and penicillin/streptomycin, in the presence of 250 μg/ml G418 (Calbiochem, San Diego, Calif.) and 1 μg/ml of blasticidin S.

### Example 3

### Synthesis of dsRNA and siRNA

Drosophila presenilin, nicastrin, PSF, and other candidate cDNA fragments, approximately 700 bp in length, were reamplified, first, by PCR using a forward primer containing a 5′ T7 RNA polymerase binding site (GAATTAATACGACTCACTATAGGG AGA (SEQ ID NO:30)) and a reverse primer containing a 3′ SP6 RNA polymerase binding site (ATTTAGGTGACACTATAGAAGCG (SEQ ID NO:31)), and, subsequently, by the specific sequences of the targeted gene. Primers used in this reaction, without T7 and SP6 sequences, are listed in Table 1.

TABLE 1
List of genes tested for their effects on Aβ generation.

| | RNAi | | |
|---|---|---|---|
| | Effects on | | |
| | Aβ | | |
| Target Proteins | Generation | Forward Primers | Reverse Primers |
| PS | Decreased | gggctcgcctctgaggatgacgccaatgtgg | aatgcggaggggtcctcttggcgaaaggacag |
| | | (SEQ ID NO:32) | (SEQ ID NO:33) |
| Nicastrin | Decreased | atggaaatgcgtctgaatgcggcttccatatggc | ttcgtatggactgtctccaaagttgtttccg |
| (AF240470) | | (SEQ ID NO:34) | (SEQ ID NO:35) |
| Human APP-C99 | Decreased | gatgcagaatccgacatgactcaggatatgaag | gttctgcatctgctcaaagaacttgtaggttgg |
| | | (SEQ ID NO:36) | (SEQ ID NO:37) |
| α-tubulin | No effect | cacattggccaagctggtgtccagatcgg | gcgcaacgaggccgtaatggaggaaacg |
| (CG9476) | | (SEQ ID NO:38) | (SEQ ID NO:39) |
| Xylulokinase | No effect | cagctgaaacgaacctttcttggcttcgac | caaatcgggtgcacaaacatttaggcattc |
| (CG3534) | | (SEQ ID NO:40) | (SEQ ID NO:41) |
| Skip | No effect | gctatgtcgctatcaagcctgctgcccacgc | gaatgtactgggcgggacccgccttctgggcatg |
| (X64536) | | (SEQ ID NO:42) | (SEQ ID NO:43) |
| Methyltransferase | No effect | cggatgctgacgacgtcttcaagcacaatgcttg | ccaggtcgtatcgtccgtagtcgcggaagaggag |
| (CG13929) | | (SEQ ID NO:44) | (SEQ IDNO:45) |
| Bleomycin hydrolase | No effect | ttaaatatgtctgacaacaacagcggatccggag | ttaacataacgctcatagaactccagcgaactaac |
| (CG1440) | | (SEQ ID NO:46) | (SEQ ID NO:47) |
| Sel-10 | No effect | ccggaaatatcatcatttcaggcagc | gtcgaggaccattagctttgtttcctc |
| (CG15010) | | (SEQ IDNO:48) | (SEQ IDNO:49) |
| β-catenin | No effect | tcgcataataatcaatacaatccacctg | gatgccgccactcttgaagatggccag |
| (CG11579) | | (SEQ ID NO:50) | (SEQ ID NO:51) |
| PSF (Drosophila | Decreased | gatgacgttgcccgagttctttggctgcaccttc | aatactaggagtatgtttactggcatgttatg |
| Aph-1) | | (SEQ ID NO:52) | (SEQ ID NO:53) |
| (CG2855, AF508786, | | | |
| AAF51212) | | | |
| Each primer was used to generate target dsRNA. A detailed method for generating dsRNA is fully described below. Forward primers were fused to T7 sequences (taatacgactcactatagggaga (SEQ ID NO:54)), and the reverse primers were fused to SP6 sequences (atttaggtgacactatagaagcg (SEQ ID NO:55)). All target proteins are | | | |
| # Drosophila forms except dsRNA directed against human APP-C99. | | | |

Sense and antisense RNAs were produced using MEGASCRIPT T7 and an SP6 high-yield transcription RNA kit (Ambion, Austin, Tex.). The generated sense and antisense RNAs were annealed by incubation at 65° C. for 30 min, then cooled at 37° C. The dsRNA products were ethanol-precipitated, resuspended in nuclease-free water, and analyzed on 1% agarose gel electrophoresis. Small interference RNA (siRNA) was synthesized by IBA GmbH, Inc. (Goettingen, Germany). The synthetic RNA oligonucleotides described below were gel-purified after deprotection, and used to generate an siRNA duplex, as previously described (Elbashir et al., Duplexes of 21-nucleotide RNAs mediate RNA interference in cultured mammalian cells. Nature, 411:494-98, 2001): (1)

(SEQ ID NO:56)

(1) sense PS1RNA:
5′-GCC/AUC/AUG/AUC/AGU/GUC/AUU/AA-3′;

(SEQ ID NO:57)

(2) antisense PS1RNA:
5′-AAU/GAC/ACU/GAU/CAU/GAU/GGC/UG-3′;

(SEQ ID NO:58)

(3) sense PS2RNA:
5′-CAC/CCU/CAU/CAU/GAU/CAG/CGU/AA-3′;

(SEQ ID NO:59)

(4) antisense PS2RNA:
5′-ACG/CUG/AUC/AUG/AUG/AGG/GUG/UG-3′;

(SEQ ID NO:60)

(5) sense nicastrin RNA:
5′-CCU/GCU/CAA/CGC/CAC/UCA/UCA/AA-3′;

(SEQ ID NO:61)

(6) antisense nicastrin RNA:
5′-UGA/UGA/GUG/GCG/UUG/AGC/AGG/UG-3′;

(SEQ ID NO:62)

(7) sense β-catenin RNA:
5′-UAG/AUG/AGG/GCA/UGC/AGA/UCC/AA-3′;

(SEQ ID NO:63)

(8) antisense β-catenin RNA:
5′-GGA/UCU/GCA/UGC/CCU/CAU/CUA/UG-3′;

(SEQ ID NO:64)

(9) sense PSF RNA:
5′-CGG/CUG/CAC/UUU/CGU/CGC/GUU/AA-3′;

(SEQ ID NO:65)

(10) antisense PSF RNA,
5′-AAC/GCG/ACG/AAA/GUG/CAG/CCG/UG-3′;

(SEQ ID NO:66)

(11) sense PSFL RNA:
5′-CGU/CCC/UUG/UUU/GGU/UCA/UGG/AA-3′;
and

(SEQ ID NO:67)

(12) antisense PSFL RNA:
5′-CCA/UGA/ACC/AAA/CAA/GGG/ACG/UG-3′.

### Example 4

### RNA Interference and RT-PCR Analysis

S2 cells (approximately 3×10⁶) were cultured in 6-well plates using Drosophila expression system (DES) serum-free medium (Invitrogen Corporation, Carlsbad, Calif.). RNA interference experiments were performed by adding dsRNA to the medium mixed with 7 μl of Lipofectamine 2000 (Invitrogen), in accordance with the manufacturer's instruction. Cells were left for 30 min at room temperature, and then were incubated for 3 days at 25° C. To ensure the effect of dsRNA, each RNA was purified with RNeasy Mini Kit (Qiagen, Valencia, Calif.), and RT PCR was performed by SUPERSCRIP™ One-Step RT-PCR with PLATINUM® Taq (Invitrogen). Transfection of siRNAs for target endogenous genes was carried out using Oligofectamine (Invitrogen) (Elbashir et al., Duplexes of 21-nucleotide RNAs mediate RNA interference in cultured mammalian cells. Nature, 411:494-98, 2001). HEK293 cells were transfected with 25 nM of siRNA duplex. After 60 h, cells were harvested and subjected to Western blotting.

### Example 5

### Analysis of hPSF mRNA Expression

Human RAPID-SCAN™ gene expression and human brain RAPID-SCAN™ gene expression panels (OriGene Technologies, Inc., Rockville, Md.) were used to examine the tissue distribution and the spliced product of hPSF. The sets of β-actin primers provided by OriGene were used as an internal control, and the sets of hPSF flanking primers were as follows: (1) HPSF1(S1): CCACCCCCCTTCCCACCTGACCAGCCATGG (SEQ ID NO:68); and (2) HPSF1(A1): CACTGTCCAGAACTGGAGATGGAGAAATAC (SEQ ID NO:69). The PCR schedule consisted of the HotStartTaq polymerase activation at 94° C. for 10 min, followed by denaturation at 94° C. for 1 min, primer extension at 62° C. for 1 min, and primer extension at 72° C. for 2.5 min. One of the amplified cDNAs was cloned with TOPO TA cloning kit, and the sequence was verified.

### Example 6

### Western Blot Analysis and Affinity Purification

At 72 h post-transfection, S2 cells were lysed using buffer A (100 mM Tris-HCl, pH 7.4; 150 mM NaCl; 1% Triton X-100; 0.25% Nonidet P-40; 2 mM EDTA) supplemented with protease inhibitor cocktail tablet (Roche Molecular Biochemicals, Indianapolis, Ind.). Secreted Aβ was detected in conditioned DES medium after concentrating the medium with Speed Vac.
hPSF-expressed and vector-expressed APP-293 cells cultured in a 100 mm-dish were lysed in buffer B (100 mM Tris-HCl, pH 7.4; 150 mMNaCl, 1% CHAPSO) supplemented with protease inhibitor cocktail tablet. After centrifugation at 14.000×g for 10 min, the supernatant was collected. 20 μl of talon metal affinity resin (Clontech, Palo Alto, Calif.) equilibrated with buffer B was added to the supernatant, and incubated for 30 min at 4° C. After the precipitates were washed four times with buffer B, the protein complex was eluted with buffer B containing 150 mM imidazole. The eluted protein complex was mixed with 2×NuPage™ LDS sample buffer (Invitrogen Corporation, Carlsbad, Calif.), and subjected to Western blotting. Western-blot experiments were carried out using either NuPAGE™ 4-12% Bis-Tris Gel or 4-20% Tris-Glycine Gel (Invitrogen). Primary antibodies were used at the following dilutions: R1 at 1:5,000; anti-V5 (Invitrogen) at 1:1000; anti-HA (HA 11, Covance) at 1:1000; 6E10 (Signet) at 1:10,000; anti-PS1Loop at 1:3000 (Thinakaran et al., Endoproteolysis of presenilin 1 and accumulation of processed derivatives in vivo. Neuron, 17:181-90, 1996); anti-PS2Loop at 1:2500 (Thinakaran et al., Endoproteolysis of presenilin 1 and accumulation of processed derivatives in vivo. Neuron, 17:181-90, 1996); anti-nicastrin (Affinity Bioreagents, Golden, Colo.) at 1:2500; and anti-β catenin (Transduction Lab) at 1:1000.

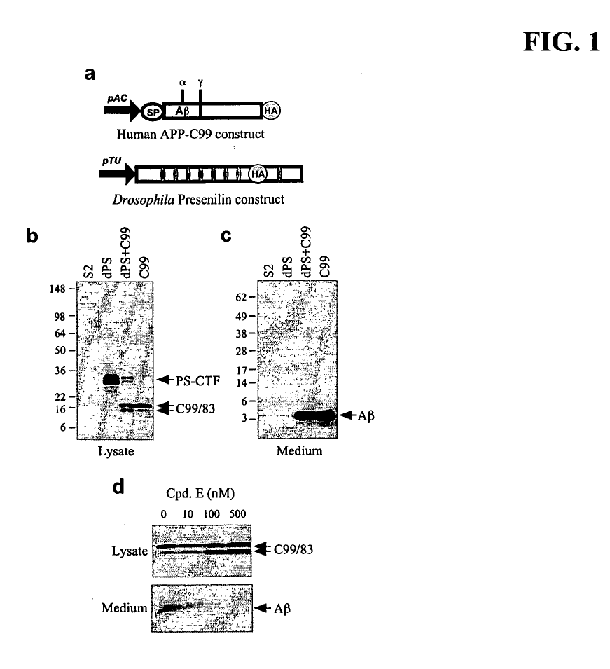

As indicated by the experiments of the above Examples, the inventors have taken a functional genetic approach to identifying genes required for γ-secretase activity and presenilin stability in vitro. In particular, the inventors developed an assay system based on double-stranded RNA interference (RNAi) in Drosophila S2 cells which are engineered to express a human γ-secretase substrate, APP-C99 (FIG. 1A). RNAi has been shown effectively and specifically to silence the expression of target genes in S2 cells (Hammond et al., An RNA-directed nuclease mediates post-transcriptional gene silencing in Drosophila cells. Nature, 404:293-96, 2000; Caplen et al., dsRNA-mediated gene silencing in cultured Drosophila cells: a tissue culture model for the analysis of RNA interference. Gene, 252:95-105, 2000; Worby et al., RNA interference of gene expression (RNAi) in cultured Drosophila cells. Sci. STKE, 95:PL1, 2001; Elbashir et al., Functional anatomy of siRNAs for mediating efficient RNAi in Drosophila melanogaster embryo lysate. EMBO J, 20:6877-88, 2001; Elbashir et al., RNA interference is mediated by 21- and 22-nucleotide RNAs. Genes Dev., 15:188-200, 2001; Fire, A. RNA-triggered gene silencing. Trends Genet., 15:358-63, 1999), thereby providing an opportunity either to test the “loss-of-function” phenotype of candidate genes, or to identify novel modulatory genes involved in γ-secretase machinery.

In the present study, stable S2 transfectants harboring constructs encoding human APP-C99 (with an N-terminal signal peptide sequence) efficiently processed C99 to generate and secrete Aβ into the medium (FIG. 1C). In these cells, transgene-derived Drosophila presenilin (dPS) was efficiently processed, and accumulated mainly as endoproteolytic fragments (FIG. 1B). Presenilin-directed γ-secretase inhibitor, Compound E (Seiffert et al., Presenilin-1 and -2 are molecular targets for gamma-secretase inhibitors. J. Biol. Chem., 275:34086-091, 2000), effectively blocked the generation of Aβ, and induced the correlated accumulation of the APP C-terminal fragments (FIG. 1D).

Nicastrin and presenilins are presently the only known components to be genetically and biochemically associated with γ-secretase activity (Chung and Struhl, Nicastrin is required for Presenilin-mediated transmembrane cleavage in Drosophila. Nature Cell Biol., 3:1129-32, 2001; Hu and Fortini, Nicastrin is required for gamma-secretase cleavage of the Drosophila Notch receptor. Dev. Cell, 2:69-78, 2002; Lopez-Schier and St. Johnston, Drosophila nicastrin is essential for the intramembranous cleavage of notch. Dev. Cell, 2:79-89, 2002; Yu et al., Nicastrin modulates presenilin-mediated notch/glp-1 signal transduction and betaAPP processing. Nature, 407:48-54, 2000; Li et al., Photoactivated gamma-secretase inhibitors directed to the active site covalently label presenilin 1. Nature, 405:689-94, 2000; Esler et al., Transition-state analogue inhibitors of gamma-secretase bind directly to presenilin-1. Nature Cell Biol., 2:428, 2000). To examine the effects of the suppression of presenilin and nicastrin on Aβ generation in S2 cells, the inventors synthesized double-stranded RNAs (dsRNAs) of the Drosophila versions of presenilin and nicastrin (dPS and dNic, respectively).

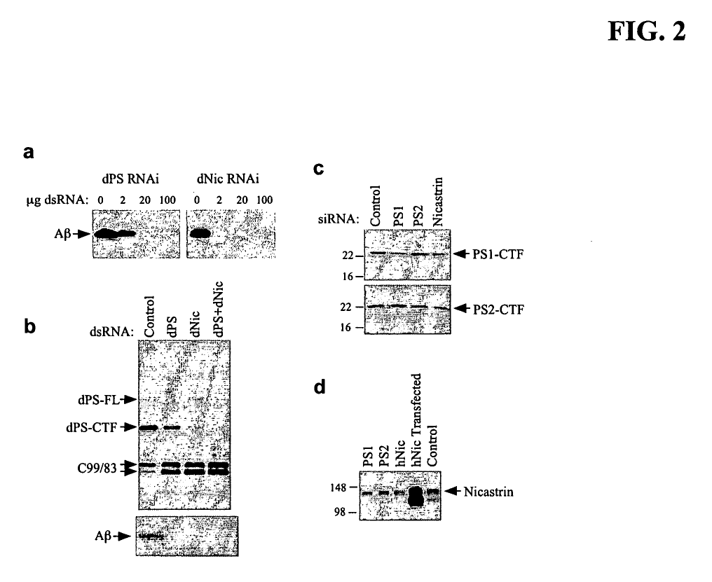

Treatment with dPS dsRNAs potently inhibited Aβ generation (FIG. 2A), and increased the accumulation of C99 (FIG. 2B) in cells stably transfected with HA-tagged versions of dPS and C99 (FIG. 1A). RNAi of dPS also reduced the amounts of dPS CTF and dPS mRNA levels (˜70% reduction, data not shown). The residual dPS-CTF immunoreactivities were likely due to the long half-life of presenilin heterodimers, as previously shown for mammalian presenilins (Thinakaran et al., Evidence that levels of presenilins (PS1 and PS2) are coordinately regulated by competition for limiting cellular factors. J. Biol. Chem., 272:28415-422, 1997; Tomita et al., The first proline of PALP motif at the C terminus of presenilins is obligatory for stabilization, complex formation, and gamma-secretase activities of presenilins. J. Biol. Chem., 276:33273-281, 2001; Kim et al., Endoproteolytic processing and proteasomal degradation of presenilin 2 in transfected cells. J. Biol. Chem., 272:11006-010, 1997), since the immature, full-length form (dPS-FL) was undetectable upon dPS dsRNA treatment (FIG. 2B).

Like dPS, dNic RNAi virtually abolished Aβ generation (FIG. 2A), thereby indicating that nicastrin is critical for presenilin-dependent γ-secretase activity. dNic RNAi also abrogated the accumulation of dPS CTF even more potently than did dPS RNAi (FIG. 2B). However, the RNAi-mediated suppression of dNic expression had no effects on the levels of full-length dPS (FIG. 2B).

The inventors further tested the hypothesis that suppression of nicastrin affects the stability of PS1 and/or PS2 in mammalian cell lines. The majority of mammalian cells are unable to convert a long dsRNA into a short, small interference RNA (siRNA) which can serve as an effector unit (21 to 23 bp) for RNAi induction (Elbashir et al., Functional anatomy of siRNAs for mediating efficient RNAi in Drosophila melanogaster embryo lysate. EMBO J, 20:6877-88, 2001; Elbashir et al., RNA interference is mediated by 21- and 22-nucleotide RNAs. Genes Dev., 15:188-200, 2001; Fire, A., RNA-triggered gene silencing. Trends Genet., 15:358-63, 1999; Elbashir et al., Duplexes of 21-nucleotide RNAs mediate RNA interference in cultured mammalian cells. Nature, 411:494-98, 2001). Therefore, the inventors designed and synthesized siRNAs (Hu and Fortini, Nicastrin is required for gamma-secretase cleavage of the Drosophila Notch receptor. Dev. Cell, 2:69-78, 2002) directed against human PS1, PS2, or nicastrin. For siRNA sequences, see Examples above.

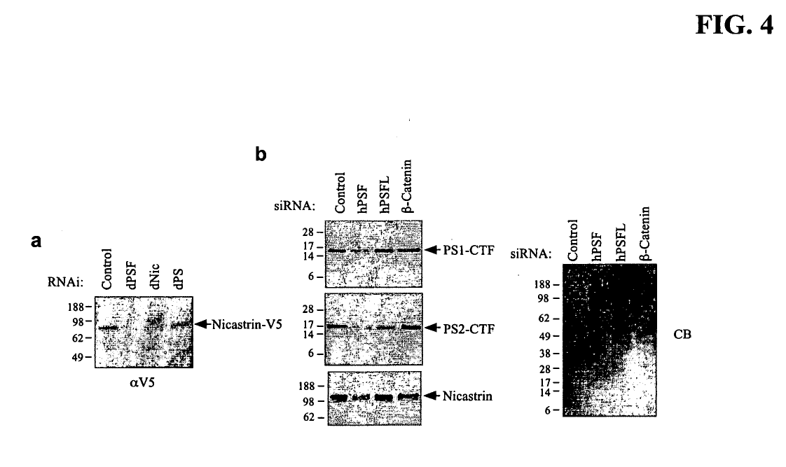

The treatment of human HEK293 cells with siRNA directed against PS1, PS2, or nicastrin resulted in the downregulation of the target proteins (FIGS. 2C and 2D). The residual immunoreactivities of the target proteins were likely due to the fact that siRNAs are only transfected in a partial population of the cells. Nicastrin siRNA transfection led to the reduced accumulations of both PS1 and PS2 (FIG. 2C), a result similar to that described in FIG. 2B. Interestingly, RNAi of PS1 or PS2 induced a reduction in the levels of nicastrin (FIG. 2D). These data suggest that presenilins and nicastrin are interdependent for their stabilization —a step that may be critical in the formation of a functional γ-secretase complex (cf FIG. 4A).

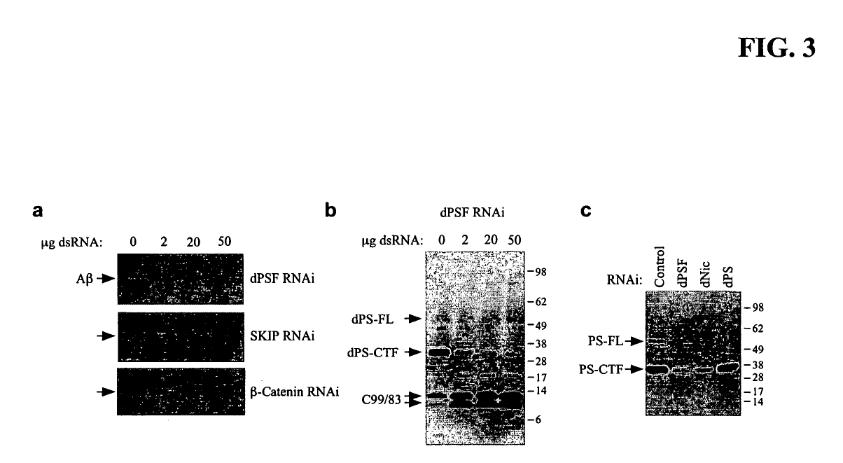

The inventors also evaluated the consequence of RNAi-mediated suppression of candidate genes on Aβ generation, including those implicated in Notch signaling, Drosophila orthologues of mammalian presenilin-interacting proteins, and genes that are genetically or biochemically implicated in Alzheimer's disease (Table 1). Inactivation of APH-1 has been shown to cause defects in the cell surface localization of APH-2 (C. elegans nicastrin orthologue), suggesting a role in protein trafficking (Goutte et al., APH-1 is a multipass membrane protein essential for the Notch signaling pathway in Caenorhabditis elegans embryos. Proc. Natl. Acad Sci, USA, 99:775-79, 2002). Among the genes that the inventors initially tested (Table 1) (Lopez-Schier and St. Johnston, Drosophila nicastrin is essential for the intramembranous cleavage of notch. Dev. Cell, 2:79-89, 2002), it was found that suppression of gene expression of a Drosophila homologue of C. elegans APH-1 conferred profound effects on Aβ generation (FIG. 3A). The inventors termed the homologue “presenilin stabilization factor”, or PSF, based on its role in presenilin stability.

RNAi-mediated suppression of Drosophila PSF(dPSF) virtually abrogated the Aβ generation in the medium (FIG. 3A), and caused a correlated increase in the levels of γ-secretase substrates, C99/83, in the lysate (FIG. 3B). These results indicate that PSF is directly involved in the γ-secretase-mediated cleavage of C99, and suggest that PSF does not affect other pathways (i.e., Aβ secretion or degradation). In contrast, RNAi of β-catenin, SKIP, and other molecules tested (Drosophila forms of β-tubulin, xylulokinase, methyltransferase, bleomycin hydrolase, and sel-10) did not confer any detectable inhibitory effects on AΔ generation (FIG. 3A; Table 1). Downregulation of the target genes was verified by RT-PCR analysis (data not shown).

To determine whether the inhibition of AΔ generation by dPSF RNAi could be attributed to the destabilization of functional γ-secretase complex, the inventors then examined the effects of dPSF RNAi on the stability of dPS in S2 cells. Treatment of stable S2 cell lines with dPSF dsRNAs selectively decreased the cellular levels of dPS-CTF (FIGS. 3B and 3C). In contrast, dPSF RNAi had no effect on full-length dPS (FIGS. 3B and 3C), and dNic RNAi had no effect on dPS-CTF (FIG. 3C). Since it has been well documented that the endoproteolytic fragments (a long-lived pool), but not full-length forms (a short-lived pool), are the functional units of the presenilins (Thinakaran et al., Evidence that levels of presenilins (PS1 and PS2) are coordinately regulated by competition for limiting cellular factors. J. Biol. Chem., 272:28415-422, 1997; Tomita et al., The first proline of PALP motif at the C terminus of presenilins is obligatory for stabilization, complex formation, and gamma-secretase activities of presenilins. J. Biol. Chem., 276:33273-281, 2001), the inventors' data suggest that dPSF plays a critical role in the stabilization of dPS CTF, but does not directly affect either the synthesis or the processing of dPS (FIGS. 3B and 3C).

Nicastrin has been shown to regulate γ-secretase activity by forming a large-molecular-weight complex with presenilins (Yu et al., Nicastrin modulates presenilin-mediated notch/glp-1 signal transduction and betaAPP processing. Nature, 407:48-54, 2000). Because it appeared that presenilins and nicastrin are mutually required for their stabilization (FIG. 2), the inventors conducted further tests to determine whether dPSF is required for the stabilization of nicastrin (e.g., dNic) in S2 cells. RNAi-mediated suppression of dPSF virtually abolished dNic immunoreactivity as efficiently as RNAi of dNic itself (FIG. 4A), indicating that dPSF is critical for nicastrin stability. Treatment of dsRNA directed against dPS substantially reduced the levels of the Drosophila version of nicastrin (dNic) in S2 cells (FIG. 4A), an effect similar to that observed when PS1 or PS2 siRNA were directed against nicastrin in human cell lines (FIG. 2D).

dPSF/APH-1 have two related human orthologues (Goutte et al., APH-1 is a multipass membrane protein essential for the Notch signaling pathway in Caenorhabditis elegans embryos. Proc. Natl. Acad. Sci. USA, 99:775-79, 2002). To evaluate the role of these orthologues in presenilin/nicastrin stability in mammalian cells, the inventors synthesized siRNAs based on unique sequences present in human PSF (GenBank accession numbers AF508787 and AAD34072) and PSF-like protein (GenBank accession numbers AF508794 and AL136671). PSF siRNA induced a selective decrease in both C-terminal PS1 endoproteolytic fragments (FIG. 4B), as well as N-terminal fragments. However, other control proteins, including β-catenin, were not affected (data not shown). PS2 CTF and nicastrin levels were also downregulated in cells treated with PSF siRNA (FIG. 4B). In contrast, PSF-like protein (PSFL) siRNA did not confer any detectable effects on PS1-CTF and nicastrin levels (FIG. 4B). PSFL siRNA treatment led to a slight reduction in PS2-CTF levels, implying a possible connection between PSFL and PS2 metabolism. Thus, the inventors' studies identified PSF, but not PSFL, as a mammalian factor required for the stabilization of both presenilins and nicastrin.

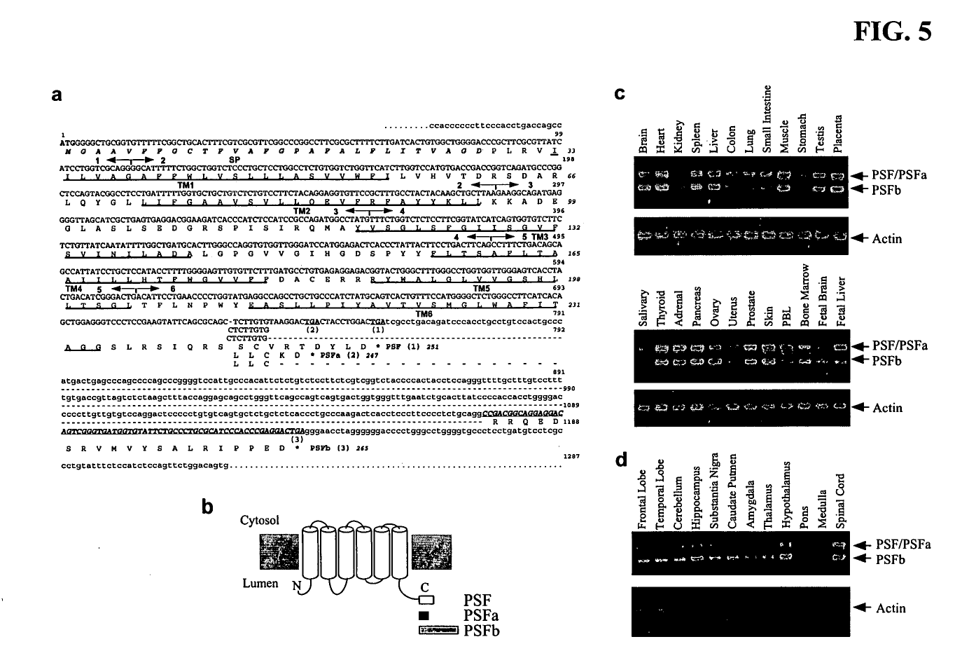

Based upon predicted PSF EST sequences in the GenBank database, human PSF cDNAs were amplified by PCR using human adult brain cDNA libraries as templates. The human PSF gene maps to a region of chromosome 1 (between D1S514 and D1S2635), where Alzheimer's disease (AD) susceptibility loci have been described (Zubenko et al., A genome survey for novel Alzheimer disease risk loci: results at 10-cM resolution. Genomics, 50:121-28, 1998; Kehoe et al., A full genome scan for late onset Alzheimer's disease. Hum. Mol. Genet., 8:237-45, 1999; Myers et al., Full genome screen for Alzheimer disease: Stage II analysis. Am. J. Med. Genet., 114:235-44, 2002), and has been predicted to contain at least 6 exons. The human PSF gene encodes an open reading frame of 251 amino acids containing a putative N-terminal signal peptide and 6 predicted transmembrane domains (FIGS. 5A and 5B).

Additionally, the inventors found the sequences of two spliced variants, resulting from either a single nucleotide insertion (PSFa) or a deletion of 338 bp (PSFb), which have been predicted to encode a protein with 247 (PSFa) or 265 (PSFb) amino acids, respectively (FIGS. 5A and 5B). The inventors isolated these alternate transcripts encoding PSF, PSFa, and PSFb, by PCR amplification using flanking primers (FIG. 5).

To determine the tissue distribution of PSF, the inventors performed RT-PCR analyses using a commercial tissue panel. Two separate primer sets were used to amplify both PSF1 and PSFa together (PSF1/PSFa) or PSFb alone (see Examples above). All PSF variants were ubiquitously expressed throughout the different human tissues tested. The level of expression of each form was similar, except for the brain, where PSFb was more abundant than the PSF1/PSFb form (FIG. 5C). Among the different brain areas examined, the spinal cord and hypothalamus showed the highest levels of expression (FIG. 5D). Apart from the pons and medulla, where neither PSFb nor PSF1/PSFa was detected, PSFb was ubiquitously expressed. The levels of expression of PSF1/PSFa were highest in the hippocampus, as well as the frontal lobe (FIG. 5D).

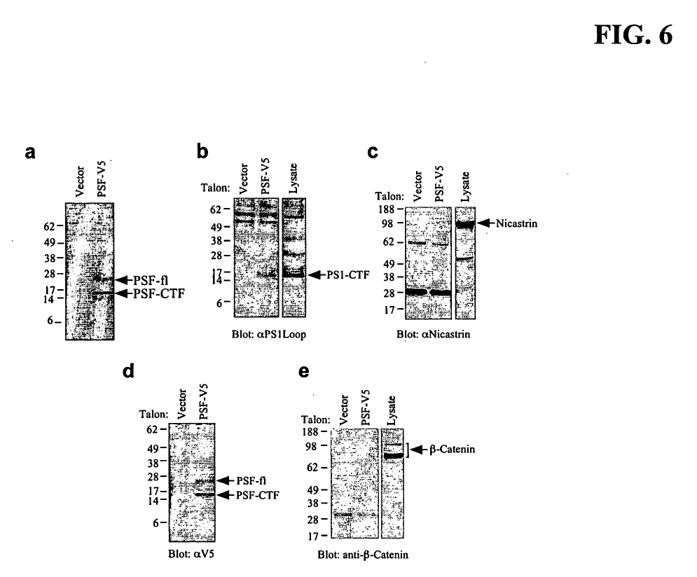

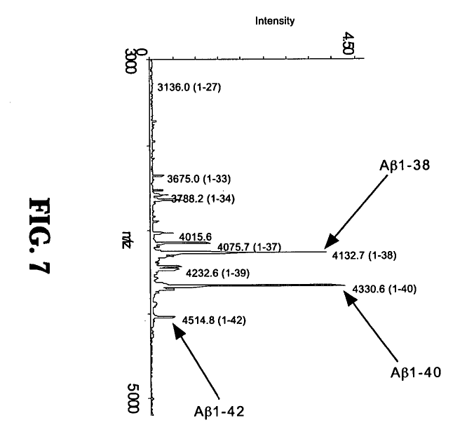

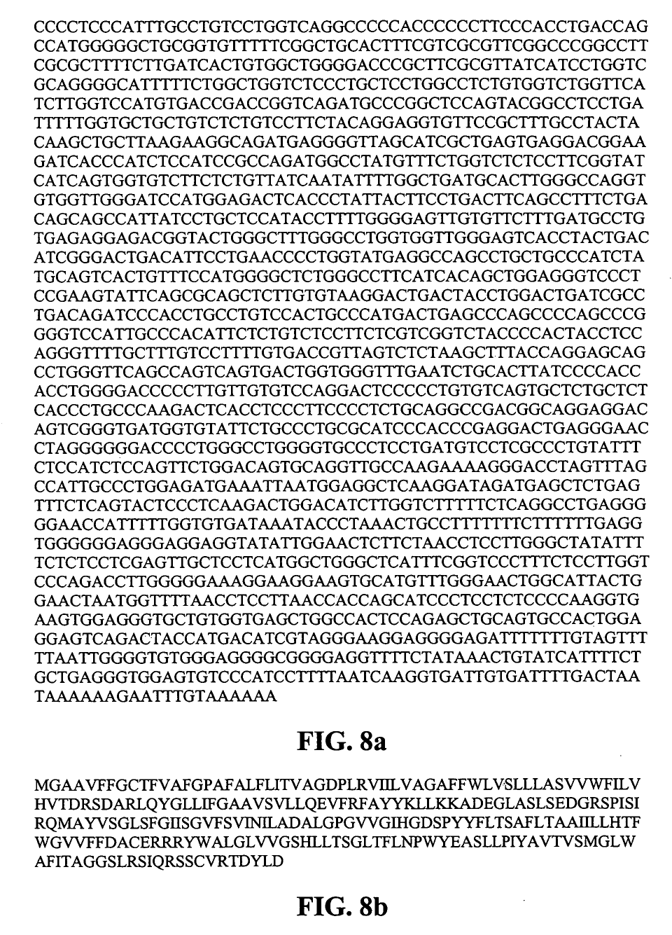

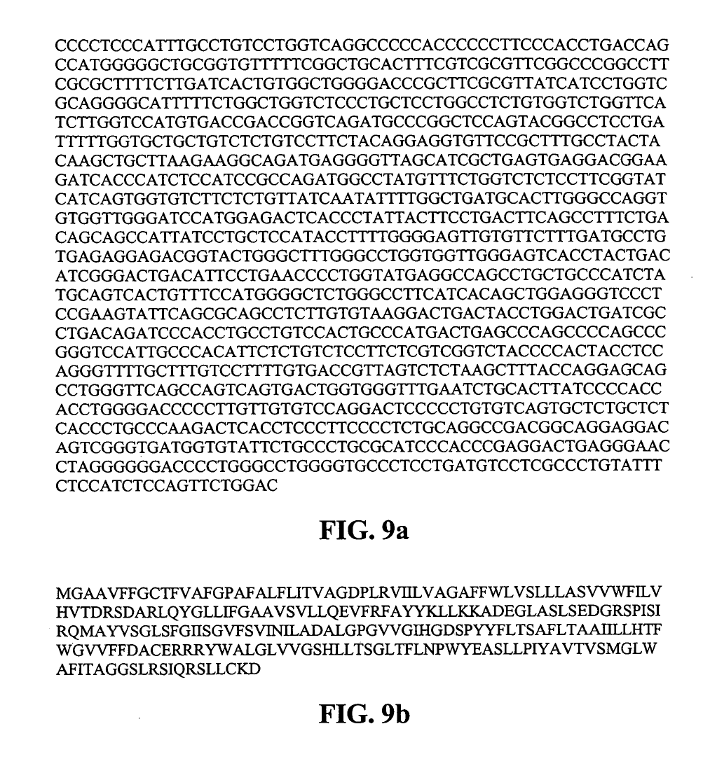

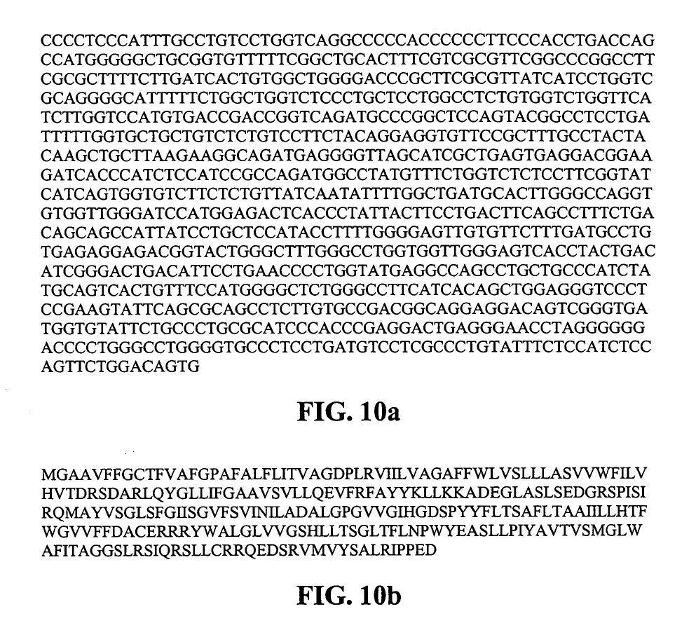

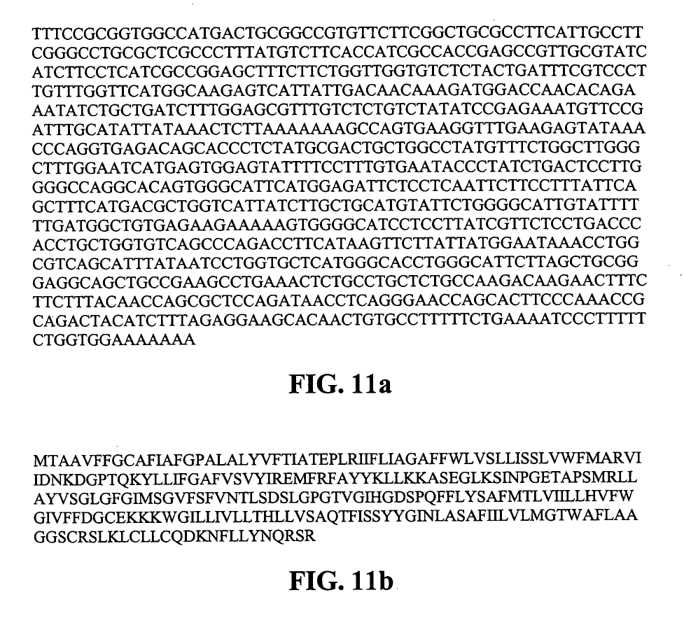

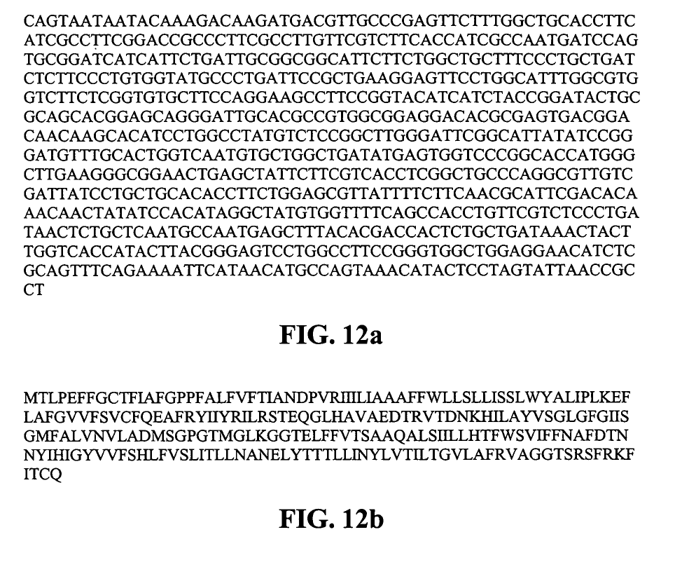

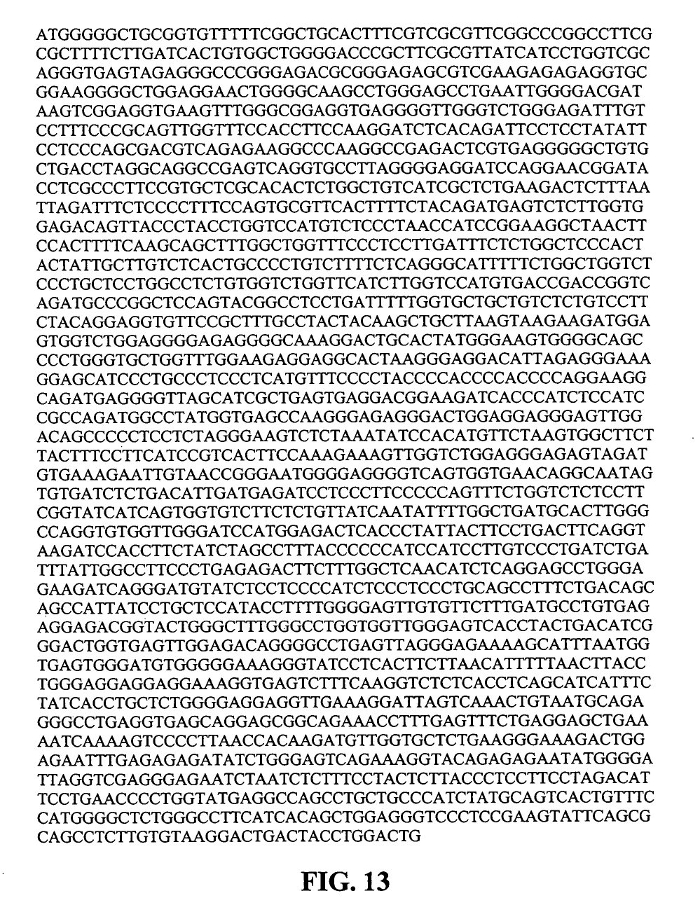

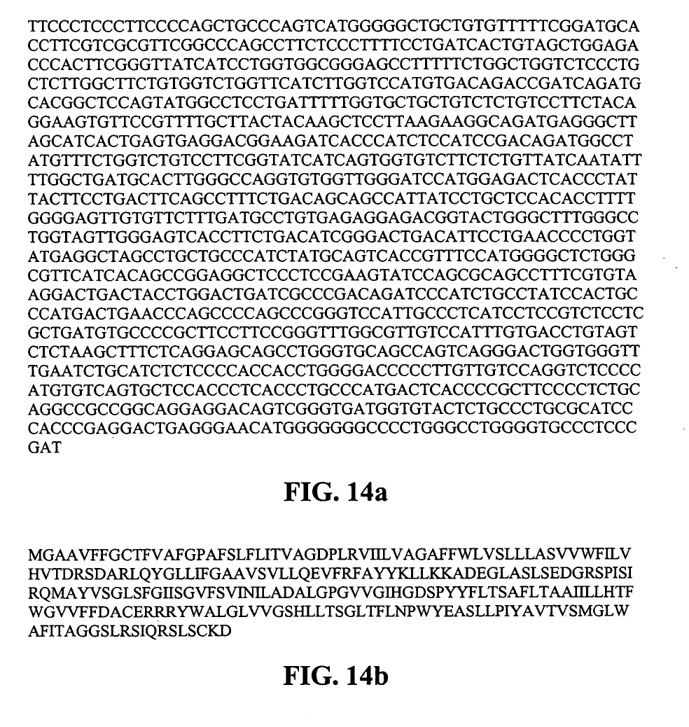

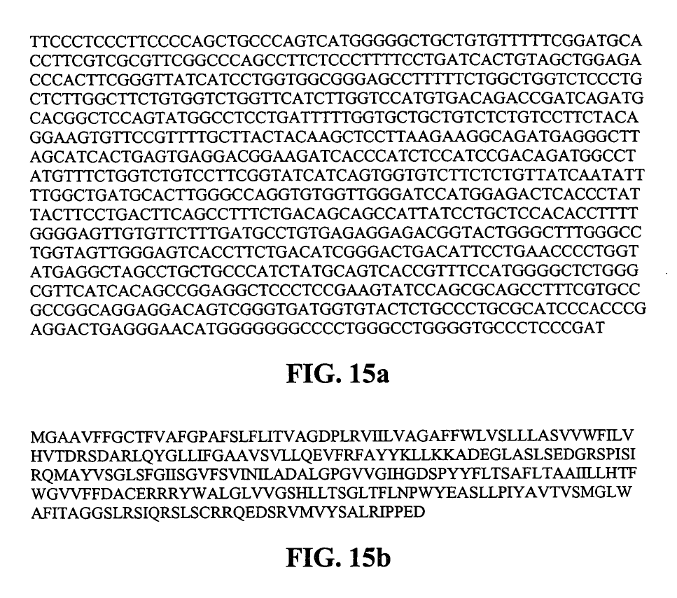

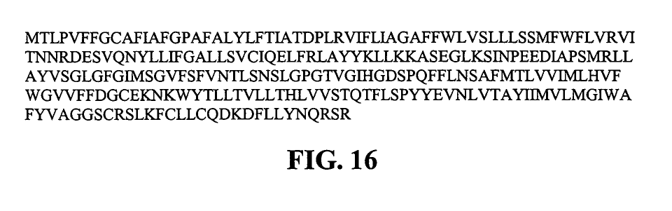

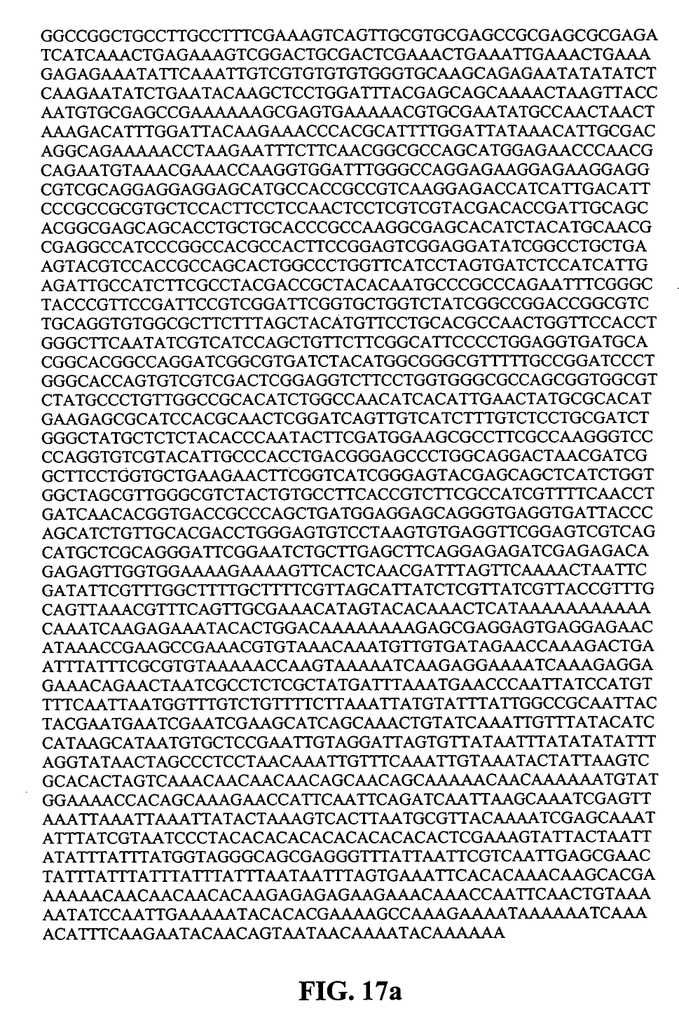

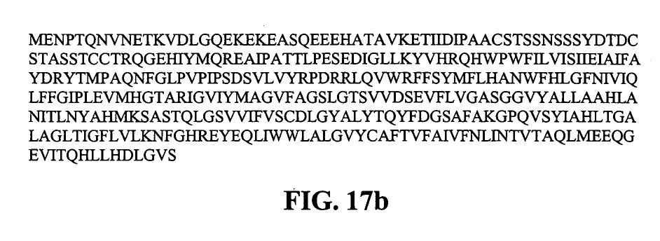

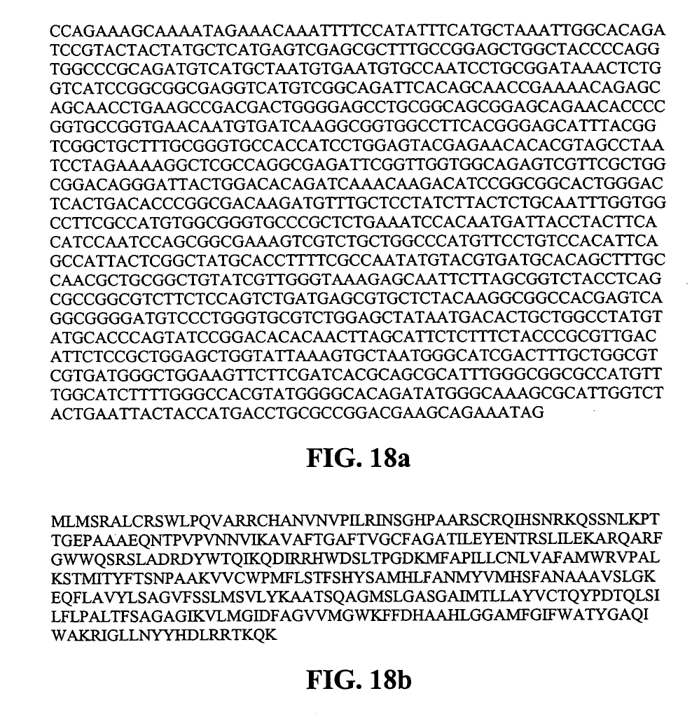

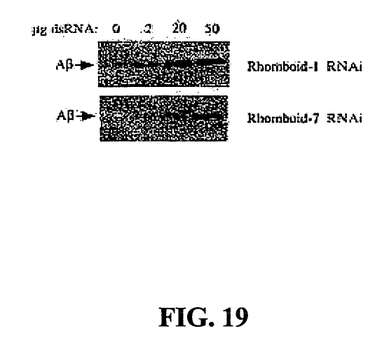

In order to characterize the PSF gene product, human full-length PSF cDNA was subcloned into an expression plasmid encoding the C-terminal, V5 and poly-histidine (6×His) tags. The resulting PSF constructs (PSF-V5) were either transiently (FIG. 6A) or stably (FIG. 6D) transfected into human HEK293 cells. Western-blot analysis using anti-V5 antibody detected two bands with apparent molecular weights of ˜25 kDa and ˜15 kDa. However, in both transient and stable expression systems, PSF was detected more prominently as a 15-kDa form (FIGS. 6A and 6D).

The foregoing data raise the possibility that, like the presenilins (Kim et al., Endoproteolytic Processing and Proteasomal Degradation of Presenilin 2 in Transfected Cells. J. Biol. Chem., 272:11006-010, 1997; Thinakaran et al., Endoproteolysis of presenilin 1 and accumulation of processed derivatives in vivo. Neuron, 17:181-90, 1996), PSF may undergo endoproteolytic processing to yield N- and C-terminal fragments, which could represent the protein's functional unit. In contrast with the presenilins, though, transient expression of PSF did not result in accumulation of the immature form (full-length version) of the protein (Kim et al., Endoproteolytic Processing and Proteasomal Degradation of Presenilin 2 in Transfected Cells. J. Biol. Chem., 272:11006-010, 1997; Thinakaran et al., Endoproteolysis of presenilin 1 and accumulation of processed derivatives in vivo. Neuron, 17:181-90, 1996). Expression levels of PSF in stable transfectants appeared to be regulated, since the high-expressing clonal cells undergo apparent cell death during the selection process (data not shown).

It has been shown that the mammalian forms of PS1 and PS2 interact with nicastrin (Yu et al., Nicastrin modulates presenilin-mediated notch/glp-1 signal transduction and betaAPP processing. Nature, 407:48-54, 2000). In addition, both the β- and α-secretase-processed forms of APP, as well as metalloprotease-processed membrane-tethered Notch, have been shown to form a complex with presenilins (Yu et al., Nicastrin modulates presenilin-mediated notch/glp-1 signal transduction and betaAPP processing. Nature, 407:48-54, 2000; Annaert et al., Interaction with telencephalin and the amyloid precursor protein predicts a ring structure for presenilins. Neuron, 32:579-89, 2001; Chen et al., Nicastrin binds to membrane-tethered Notch. Nat. Cell Biol., 3:751-54, 2001). Interestingly, activity-dependent affinity isolation using a transition-state analog γ-secretase inhibitor revealed that presenilins and nicastrin, as well as a major cellular y-substrate, C83 (α-secretase cleaved C-terminal APP stub), can be co-purified (Esler et al., Activity-dependent isolation of the presenilin-γ-secretase complex reveals nicastrin and a gamma substrate. Proc. Natl. Acad. Sci. USA, 99:2720-25, 2002), suggesting the formation of an usual substrate-protease complex.

To begin to elucidate the molecular interaction(s) underlying the PSF function, the inventors attempted to determine whether the PSF-containing molecular complex harbors presenilins or nicastrin. HEK293 cells stably expressing either vector alone or V5/polyhistidine(6×His)-tagged hPSF were lysed, and subjected to an affinity isolation using cobalt-affinity resins (Talon). Both full-length and processed forms of hPSF were isolated during this step (FIG. 6D). Endogenous forms of PS1 C-terminal fragments (FIG. 6B), as well as N-terminal fragments (data not shown), were co-purified with PSF, indicating that PSF forms a complex with endogenous PS1 (FIG. 6B). Nicastrin was also selectively co-purified with PSF (FIG. 6C). Detection of PS1 and nicastrin in the complex did not result from non-specific binding to the metal-affinity beads, since PS1 and nicastrin failed to co-purify in vector-transfected cells (FIG. 6). In addition, other proteins, such as β-catenin (FIG. 6E), were not co-isolated during this procedure. Thus, the inventors' data demonstrate the existence of a molecular complex involving presenilin, nicastrin, and PSF.

In accordance with the present invention, the inventors have demonstrated that both Drosophila and human PSF are cellular factors that play a critical role in γ-secretase activity, and in presenilin and nicastrin stability. Furthermore, the inventors have provided evidence that PSF forms a complex with other core γ-secretase components, including presenilin and nicastrin, thereby suggesting that PSF may be directly involved in the catalytic activity of the γ-secretase complex. Thus, the formation of a ternary complex composed of presenilin, nicastrin, and PSF may be needed for both catalytic activity and stabilization of a functional γ-secretase complex. Alternatively, PSF might be required for the assembly of the presenilin/nicastrin-bearing complex, or for modulating intracellular trafficking of a functional γ-secretase complex (Naruse et al., Effects of PS1 deficiency on membrane protein trafficking in neurons. Neuron, 21:1213-21, 1998; Cupers et al., The discrepancy between presenilin subcellular localization and gamma-secretase processing of amyloid precursor protein. J. Cell Biol., 154:731-40, 2001; Armogida et al. Endogenous β-amyloid production in presenilin-deficient embryonic mouse fibroblasts. Nat. Cell Biol., 3:1030-33, 2001; Taniguchi et al., Notch receptor cleavage depends on but is not directly executed by presenilins. Proc. Natl. Acad. Sci. USA, 99:4014-19, 2002)-roles that have been widely postulated for presenilin and nicastrin (Naruse et al., Effects of PS1 deficiency on membrane protein trafficking in neurons. Neuron, 21:1213-21, 1998; Cupers et al., The discrepancy between presenilin subcellular localization and gamma-secretase processing of amyloid precursor protein. J. Cell Biol., 154:731-40, 2001).

While the foregoing invention has been described in some detail for purposes of clarity and understanding, it will be appreciated by one skilled in the art, from a reading of the disclosure, that various changes in form and detail can be made without departing from the true scope of the invention in the appended claims.

1. An isolated nucleic acid sequence encoding a polypeptide, wherein the polypeptide is selected from the group consisting of presenilin stabilization factor (PSF) and PSF-like protein (PSFL).

2. The nucleic acid sequence of claim 1, which is DNA or RNA.

3. The nucleic acid sequence of claim 1, comprising a nucleotide sequence selected from the group consisting of SEQ ID NOs: 1, 2, 3, 7, 9, 11, 13, 15, 17, 18, and 20.

4. The nucleic acid sequence of claim 1, wherein the polypeptide comprises an amino acid sequence selected from the group consisting of SEQ ID NOs: 4, 5, 6, 8, 10, 12, 14, 16, 19, 21, and 70.

5. The nucleic acid sequence of claim 1, which encodes human PSF.

6. An isolated nucleic acid sequence that hybridizes under high-stringency conditions to a second nucleic acid sequence, wherein the second nucleic acid is complementary to a nucleotide sequence selected from the group consisting of SEQ ID NOs: 1, 2, 3, 7, 9, 11, 13, 15, 17, 18, and 20, or to a continuous fragment thereof.

7. A purified polypeptide, selected from the group consisting of presenilin stabilization factor (PSF) and PSF-like protein (PSFL).

8. The polypeptide of claim 7, comprising an amino acid sequence selected from the group consisting of SEQ ID NOs: 4, 5, 6, 8, 10, 12, 14, 16, 19, 21, and 70.

9. The polypeptide of claim 7, which is encoded by a nucleotide sequence selected from the group consisting of SEQ ID NOs: 1, 2, 3, 7, 9, 11, 13, 15, 17, 18, and 20.

10. The polypeptide of claim 7, which is human PSF.

11. A purified polypeptide encoded by a nucleic acid sequence that hybridizes under high-stringency conditions to a second nucleic acid sequence, wherein the second nucleic acid sequence is complementary to a nucleotide sequence selected from the group consisting of SEQ ID NOs: 1, 2, 3, 7, 9, 11, 13, 15, 17, 18, and 20, or to a continuous fragment thereof.

12. A pharmaceutical composition, comprising a pharmaceutically-acceptable carrier and presenilin stabilization factor (PSF) or presenilin stabilization factor-like protein (PSFL).

13. An antibody specific for a polypeptide, wherein the polypeptide is selected from the group consisting of presenilin stabilization factor (PSF) and PSF-like protein (PSFL).

14. The antibody of claim 13, wherein the polypeptide is human PSF.

15. A method for producing an antibody specific for a polypeptide selected from the group consisting of presenilin stabilization factor (PSF) and PSF-like protein (PSFL), comprising the steps of:
(a) immunizing a mammal with the selected polypeptide; and
(b) purifying antibody from a tissue of the mammal or from a hybridoma made using tissue of the mammal.

16. The method of claim 15, wherein the polypeptide is human PSF.

17. An antibody produced by the method of claim 15.

18. A vector comprising a nucleic acid sequence encoding a polypeptide, wherein the polypeptide is selected from the group consisting of presenilin stabilization factor (PSF) and PSF-like protein (PSFL).

19. The vector of claim 18, wherein the nucleic acid sequence comprises a nucleotide sequence selected from the group consisting of SEQ ID NOs: 1, 2, 3, 7, 9, 11, 13, 15, 17, 18, and 20.

20. The vector of claim 18, wherein the nucleic acid sequence hybridizes under high-stringency conditions to a second nucleic acid sequence, wherein the second nucleic acid sequence is complementary to a nucleotide sequence selected from the group consisting of SEQ ID NOs: 1, 2, 3, 7, 9, 11, 13, 15, 17, 18, and 20, or to a continuous fragment thereof.

21. The vector of claim 18, wherein the nucleic acid sequence encodes human PSF.

22. A host cell transformed with the vector of claim 18.

23. A transgenic animal containing the host cell of claim 22.

24. A method for making a polypeptide selected from the group consisting of presenilin stabilization factor (PSF) and PSF-like protein (PSFL), comprising the steps of:
(a) introducing into a host cell a nucleic acid sequence encoding the selected polypeptide;
(b) maintaining the host cell under conditions such that the nucleic acid sequence is expressed to produce the selected polypeptide; and
(c) recovering the selected polypeptide.

25. The method of claim 24, wherein the polypeptide is human PSF.

26. A method for decreasing amyloid-beta production in a cell, comprising decreasing activity of a presenilin-stabilizing molecule in the cell, wherein the molecule is selected from the group consisting of presenilin stabilization factor (PSF) and presenilin stabilization factor-like protein (PSFL).

27. The method of claim 26, wherein the molecule decreases amyloid-beta production in the cell by a biological process selected from the group consisting of:
(a) destabilizing presenilin or nicastrin in the cell;
(b) destabilizing a gamma-secretase complex in the cell; and
(c) inhibiting activity of gamma-secretase in the cell.

28. The method of claim 26, wherein activity of the molecule is decreased in the cell by contacting the cell with an inhibitor of the molecule.

29. The method of claim 28, wherein the cell is contacted with an amount of the inhibitor effective to decrease amyloid-beta production in the cell.

30. The method of claim 28, wherein the inhibitor is dsRNA.

31. The method of claim 28, wherein the contacting is effected in vitro.

32. The method of claim 28, wherein the contacting is effected in vivo in a subject.

33. The method of claim 32, wherein the contacting is effected in vivo in a subject by administering the inhibitor to the subject.

34. The method of claim 33, wherein the inhibitor is administered to the subject by introducing the inhibitor into cells of the subject.

35. The method of claim 34, wherein the inhibitor is introduced into cells of the subject by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with recombinant replication-defective viruses, homologous recombination, in vivo gene therapy, ex vivo gene therapy, viral vectors, and naked DNA transfer.

36. The method of claim 33, wherein the inhibitor is administered to the subject by oral administration, parenteral administration, transdermal administration, or osmotic pump.

37. The method of claim 32, wherein the subject is a human.

38. The method of claim 37, wherein the human has neurodegeneration.

39. The method of claim 38, wherein the neurodegeneration is selected from the group consisting of Alzheimer's disease, amyotrophic lateral sclerosis (Lou Gehrig's Disease), Binswanger's disease, corticobasal degeneration (CBD), dementia lacking distinctive histopathology (DLDH), frontotemporal dementia (FTD), Huntington's chorea, multiple sclerosis, myasthenia gravis, Parkinson's disease, Pick's disease, and progressive supranuclear palsy (PSP).

40. The method of claim 39, wherein the neurodegeneration is Alzheimer's disease.

41. The method of claim 40, wherein the Alzheimer's disease is early-onset familial Alzheimer's disease.

42. A pharmaceutical composition for decreasing amyloid-beta production, comprising a pharmaceutically-acceptable carrier and an inhibitor of a presenilin-stabilizing molecule, wherein the molecule is selected from the group consisting of presenilin stabilization factor (PSF) and presenilin stabilization factor-like protein (PSFL).

43. A method for destabilizing presenilin or nicastrin in a cell, comprising decreasing activity of a presenilin-stabilizing molecule in the cell, wherein the molecule is selected from the group consisting of presenilin stabilization factor (PSF) and presenilin stabilization factor-like protein (PSFL).

44. A method for destabilizing a gamma-secretase complex in a cell, comprising decreasing activity of a presenilin-stabilizing molecule in the cell, wherein the molecule is selected from the group consisting of presenilin stabilization factor (PSF) and presenilin stabilization factor-like protein (PSFL).

45. A method for inhibiting activity of gamma-secretase in a cell, comprising decreasing activity of a presenilin-stabilizing molecule in the cell, wherein the molecule is selected from the group consisting of presenilin stabilization factor (PSF) and presenilin stabilization factor-like protein (PSFL).

46. A method for decreasing amyloid-beta production in a cell, comprising increasing activity of a rhomboid peptide in the cell, wherein the peptide is selected from the group consisting of rhomboid 1 and rhomboid 7.

47. The method of claim 46, wherein activity of the peptide is increased in the cell by contacting the cell with the peptide or a modulator of the peptide's expression.

48. The method of claim 47, wherein the cell is contacted with an amount of the peptide or modulator effective to decrease amyloid-beta production in the cell.

49. The method of claim 47, wherein the contacting is effected in vitro.

50. The method of claim 47, wherein the contacting is effected in vivo in a subject.

51. The method of claim 50, wherein the contacting is effected in vivo in a subject by administering the peptide or the modulator to the subject.

52. The method of claim 51, wherein the peptide or the modulator is administered to the subject by oral administration, parenteral administration, transdermal administration, or osmotic pump.

53. The method of claim 51, wherein the peptide or the modulator is administered to the subject by introducing a nucleic acid encoding the peptide or the modulator into cells of the subject, in a manner permitting expression of the peptide or the modulator.

54. The method of claim 53, wherein the nucleic acid is introduced into cells of the subject by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with recombinant replication-defective viruses, homologous recombination, in vivo gene therapy, ex vivo gene therapy, viral vectors, and naked DNA transfer.

55. The method of claim 50, wherein the subject is a human.

56. The method of claim 55, wherein the human has neurodegeneration.

57. The method of claim 56, wherein the neurodegeneration is selected from the group consisting of Alzheimer's disease, amyotrophic lateral sclerosis (Lou Gehrig's Disease), Binswanger's disease, corticobasal degeneration (CBD), dementia lacking distinctive histopathology (DLDH), frontotemporal dementia (FTD), Huntington's chorea, multiple sclerosis, myasthenia gravis, Parkinson's disease, Pick's disease, and progressive supranuclear palsy (PSP).

58. The method of claim 57, wherein the neurodegeneration is Alzheimer's disease.

59. The method of claim 58, wherein the Alzheimer's disease is early-onset familial Alzheimer's disease.

60. A pharmaceutical composition for decreasing amyloid-beta production, comprising a rhomboid peptide, or a modulator of the peptide's expression, and a pharmaceutically-acceptable carrier, wherein the peptide is selected from the group consisting of rhomboid 1 and rhomboid 7.

61. A method for treating neurodegeneration in a subject in need of treatment, comprising administering to the subject an inhibitor of a presenilin-stabilizing molecule, in an amount effective to treat the neurodegeneration, wherein the molecule is selected from the group consisting of presenilin stabilization factor (PSF) and presenilin stabilization factor-like protein (PSFL).

62. The method of claim 61, wherein the neurodegeneration is selected from the group consisting of Alzheimer's disease, amyotrophic lateral sclerosis (Lou Gehrig's Disease), Binswanger's disease, corticobasal degeneration (CBD), dementia lacking distinctive histopathology (DLDH), frontotemporal dementia (FTD), Huntington's chorea, multiple sclerosis, myasthenia gravis, Parkinson's disease, Pick's disease, and progressive supranuclear palsy (PSP).

63. The method of claim 62, wherein the neurodegeneration is Alzheimer's disease.

64. The method of claim 63, wherein the Alzheimer's disease is early-onset familial Alzheimer's disease.

65. The method of claim 61, wherein the inhibitor is dsRNA.

66. The method of claim 61, wherein the inhibitor is administered to the subject by introducing the inhibitor into cells of the subject.

67. The method of claim 66, wherein the inhibitor is introduced into cells of the subject by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with recombinant replication-defective viruses, homologous recombination, in vivo gene therapy, ex vivo gene therapy, viral vectors, and naked DNA transfer.

68. A method for treating neurodegeneration in a subject in need of treatment, comprising administering to the subject a rhomboid peptide, or a modulator of the peptide's expression, in an amount effective to treat the neurodegeneration, wherein the peptide is selected from the group consisting of rhomboid 1 and rhomboid 7.

69. The method of claim 68, wherein the neurodegeneration is selected from the group consisting of Alzheimer's disease, amyotrophic lateral sclerosis (Lou Gehrig's Disease), Binswanger's disease, corticobasal degeneration (CBD), dementia lacking distinctive histopathology (DLDH), frontotemporal dementia (FTD), Huntington's chorea, multiple sclerosis, myasthenia gravis, Parkinson's disease, Pick's disease, and progressive supranuclear palsy (PSP).

70. The method of claim 68, wherein the neurodegeneration is Alzheimer's disease.

71. The method of claim 70, wherein the Alzheimer's disease is early-onset familial Alzheimer's disease.

72. The method of claim 68, wherein the peptide or the modulator is administered to the subject by oral administration, parenteral administration, transdermal administration, or osmotic pump.

73. The method of claim 68, wherein the peptide or the modulator is administered to the subject by introducing a nucleic acid encoding the peptide or the modulator into cells of the subject, in a manner permitting expression of the peptide or the modulator.

74. The method of claim 73, wherein the nucleic acid is introduced into cells of the subject by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with recombinant replication-defective viruses, homologous recombination, in vivo gene therapy, ex vivo gene therapy, viral vectors, and naked DNA transfer.

75. An in vitro system for identifying an agent that selectively modulates production of amyloid-beta or an amyloid-beta precursor, comprising Drosophila-derived S2 cells that express human APP, a human APP derivative, or a human presenilin.

76. A method for making an in vitro system for identifying an agent that selectively modulates production of amyloid-beta or an amyloid-beta precursor, comprising the step of:
(a) generating Drosophila-derived S2 cells that express human APP, a human APP derivative, or a human presenilin.

77. An in vitro system made by the method of claim 76.

78. The in vitro system of claim 77, wherein the method further comprises the steps of:
(b) contacting the cells with dsRNA for a candidate protein product; and
(c) assessing the ability of the dsRNA to modulate production of amyloid-beta or an amyloid-beta precursor in the cells, wherein ability of the dsRNA to modulate production of amyloid-beta or an amyloid-beta precursor is indicative that the candidate protein product modulates production of amyloid-beta or an amyloid-beta precursor.

79. The in vitro system of claim 77, wherein the method further comprises the steps of:
(b) contacting the cells with a candidate agent; and
(c) assessing the ability of the candidate agent to modulate production of amyloid-beta or an amyloid-beta precursor in the cells.

80. A method for identifying a protein product that modulates production of amyloid-beta or an amyloid-beta precursor, comprising the steps of:
(a) obtaining or generating Drosophila-derived S2 cells that express human APP, a human APP derivative, or a human presenilin;
(b) contacting the cells with dsRNA for a candidate protein product; and
(c) assessing the ability of the dsRNA to modulate production of amyloid-beta or an amyloid-beta precursor in the cells, wherein ability of the dsRNA to modulate production of amyloid-beta or an amyloid-beta precursor is indicative that the candidate protein product modulates production of amyloid-beta or an amyloid-beta precursor.

81. A protein product identified by the method of claim 80.

82. The protein product of claim 81, which decreases production of amyloid-beta or an amyloid-beta precursor.

83. A method for treating neurodegeneration in a subject in need of treatment, comprising administering to the subject an amount of the protein product of claim 82 effective to treat neurodegeneration in the subject.

84. The method of claim 83, wherein the neurodegeneration is selected from the group consisting of Alzheimer's disease, amyotrophic lateral sclerosis (Lou Gehrig's Disease), Binswanger's disease, corticobasal degeneration (CBD), dementia lacking distinctive histopathology (DLDH), frontotemporal dementia (FTD), Huntington's chorea, multiple sclerosis, myasthenia gravis, Parkinson's disease, Pick's disease, and progressive supranuclear palsy (PSP).

85. The method of claim 84, wherein the neurodegeneration is Alzheimer's disease.

86. The method of claim 85, wherein the Alzheimer's disease is early-onset familial Alzheimer's disease.

87. A method for identifying an agent that modulates production of amyloid-beta or an amyloid-beta precursor, comprising the steps of:
(a) obtaining or generating Drosophila-derived S2 cells that express human APP, a human APP derivative, or a human presenilin;
(b) contacting the cells with a candidate agent; and
(c) assessing the ability of the candidate agent to modulate production of amyloid-beta or an amyloid-beta precursor in the cells.

88. An agent identified by the method of claim 87.

89. The agent of claim 88, which decreases production of amyloid-beta.

90. A method for treating neurodegeneration in a subject in need of treatment, comprising administering to the subject an amount of the agent of claim 89 effective to treat neurodegeneration in the subject.

91. The method of claim 90, wherein the neurodegeneration is selected from the group consisting of Alzheimer's disease, amyotrophic lateral sclerosis (Lou Gehrig's Disease), Binswanger's disease, corticobasal degeneration (CBD), dementia lacking distinctive histopathology (DLDH), frontotemporal dementia (FTD), Huntington's chorea, multiple sclerosis, myasthenia gravis, Parkinson's disease, Pick's disease, and progressive supranuclear palsy (PSP).

92. The method of claim 91, wherein the neurodegeneration is Alzheimer's disease.

93. The method of claim 92, wherein the Alzheimer's disease is early-onset familial Alzheimer's disease.
